(19) United States
(12) Patent Application Publication
(10) Pub. No.: US 2026/0091227 A1
(43) Pub. Date: Apr. 2, 2026

(54) Stimulation protocol for comfortable spatiotemporal transcutaneous spinal cord stimulation

(71) Applicants: Ismael Seanez, St. Louis, MO (US); Noah Bryson, St. Louis, MO (US); Lorenzo Lombardi, St. Louis, MO (US); Rachel Hawthorn, St. Louis, MO (US); Rodolfo Keesey, St. Louis, MO (US); Carolyn Atkinson, St. Louis, MO (US); Zach Seitz, St. Louis, MO (US); Meredith Lang, St. Louis, MO (US); Eric Leuthardt, St. Louis, MO (US); Peter Brunner, St. Louis, MO (US)

(72) Inventors: Ismael Seanez, St. Louis, MO (US); Noah Bryson, St. Louis, MO (US); Lorenzo Lombardi, St. Louis, MO (US); Rachel Hawthorn, St. Louis, MO (US); Rodolfo Keesey, St. Louis, MO (US); Carolyn Atkinson, St. Louis, MO (US); Zach Seitz, St. Louis, MO (US); Meredith Lang, St. Louis, MO (US); Eric Leuthardt, St. Louis, MO (US); Peter Brunner, St. Louis, MO (US)

(73) Assignee: Washington University, St. Louis, MO (US)

(21) Appl. No.: 19/279,918

(22) Filed: Jul. 24, 2025

Related U.S. Application Data
(60) Provisional application No. 63/675,101, filed on Jul. 24, 2024.

(51) Int. Cl. A61N 1/36; A61N 1/04

(52) U.S. Cl. CPC A61N 1/36003; A61N 1/0456; A61N 1/0476; A61N 1/36031; A61N 1/36034

(57) ABSTRACT

Among the various aspects of the present disclosure is the provision for systems and methods that employ a multielectrode transdermal spinal stimulation (tSCS) device configured to provide spatiotemporal transcutaneous spinal cord stimulation. In some aspects, the predetermined pattern is configured to sequentially activate and recruit individual muscle groups in a sequence configured to assist in the subject's movements such as walking. Among various other aspects a non-invasive brain-spine interface (BSI) system is disclosed that facilitate movements such as leg movements in a subject with a spinal cord injury (SCI). The BSI system includes a wearable EEG array to monitor brain activity, and a computer to identify intended movement by the subject based on the monitored brain activity. In response to the detection of an intended movement by the BSI system, the tSCS device activates the multielectrode transdermal spinal stimulation device to facilitate the intended movement of the subject.

### CROSS-REFERENCE TO RELATED APPLICATIONS

This application claims priority from U.S. Provisional Application Ser. No. 63/675,101 filed on Jul. 24, 2024, which is incorporated herein by reference in its entirety.

### STATEMENT REGARDING FEDERALLY SPONSORED RESEARCH OR DEVELOPMENT

This invention was made with government support under NS127936 awarded by the National Institutes of Health (NIH). The government has certain rights in the invention.

### MATERIAL INCORPORATED-BY-REFERENCE

Not applicable.

### FIELD OF THE INVENTION

The present disclosure generally relates to systems and methods for spatiotemporal transcutaneous spinal cord stimulation.

### BACKGROUND OF THE INVENTION

Spinal cord injury (SCI) is a life-altering event that leads to long-lasting motor impairment, and currently, there is no cure for paralysis. Spinal cord stimulation (SCS) has been gaining momentum as a neuromodulation intervention to restore movement to paralyzed areas below the injury. By selectively activating individual muscle groups at the appropriate phases of movement, epidural SCS (eSCS) can re-enable the performance of dexterous activities such as walking, cycling, swimming, and kayak paddling. However, the invasive nature of eSCS, the sizeable group of experts required to successfully administer eSCS, and the high associated costs may prevent eSCS from becoming an accessible therapy for millions of people living with paralysis.

Transcutaneous SCS (tSCS) offers a promising non-invasive alternative to engage paralyzed muscles enervated below the spinal injury by activating the same neural structures via similar mechanisms to eSCS. In conventional applications of tSCS, a single electrode is positioned over the T11/T12 interspinous ligament and centered at the midline. In clinical applications of tSCS, continuous stimulation protocols at 30 Hz or 50 Hz are used to facilitate motor function and spasticity, respectively. Recent work suggests that positive rehabilitative outcomes may also be achievable through this non-invasive approach. However, the low selectivity of tSCS compared to eSCS (FIG. 1D, FIG. 1E, FIG. 1F, FIG. 1G) may limit the types and number of movements that can be enabled by tSCS and, thus, its potential applications in exercise-based rehabilitation strategies are similarly limited.

Positioning the cathode electrode at different locations can enable the preferential recruitment of either muscles ipsilateral to the stimulation site or rostral vs caudal muscle groups in the lumbosacral region. However, repositioning an electrode to target different muscle groups within a rehabilitation session would significantly limit the feasibility of performing this approach in a clinical setting.

Multielectrode arrays positioned over the cervical spinal cord have the potential to preferentially target specific motor neuron pools. It may also be possible to attain preferential activation of specific motor neuron pools in the lumbar spinal cord using one single multielectrode configuration. However, unlike the cervical spinal cord, where the posterior roots for flexor and extensor muscles are generally separated, there is a significant overlap of posterior roots in the lumbar spinal cord. A comprehensive investigation of the improvements in selectivity enabled by multielectrode arrays positioned at the lumbar spinal cord would yield valuable information to guide the design of tSCS systems and methods. While preferential activation of specific muscle groups has been reported, there is a lack of direct comparisons to quantify the magnitude of these improvements in recruitment selectivity compared to conventional tSCS approaches or different electrode configurations. Additionally, a current limitation in the field of tSCS is that high currents are needed to recruit both leg and arm muscles. Turning the stimulation from off to on is not feasible, as people feel pain and discomfort from this sudden and drastic change in stimulation amplitude. In some aspects, the methods disclosed herein gradually increase the stimulation amplitude (over about 2 minutes) from 0 milliamps (mA) to a baseline stimulation amplitude that will be used for therapy (effective amplitude, say 100 mA). This stimulation amplitude is left at that baseline level throughout therapy for the non-activated electrodes.

Overlapping motor neuron pools for antagonist (muscles performing an action) and antagonist muscles (muscles acting against the muscle performing the action) poses an additional challenge to the development of tSCS systems and methods. By way of non-limiting example, the same electrode location that is selective for the ankle rather than the hip may activate both the ankle extensor and flexor muscles, hampering net movement of the ankle as desired.

### SUMMARY OF THE INVENTION

Among the various aspects of the present disclosure is the provision for methods of comfortable spatiotemporal transcutaneous spinal cord stimulation.

Briefly, therefore, the present disclosure is directed to devices, systems, and methods of spatiotemporal multielectrode transcutaneous spinal cord stimulation using a multielectrode stimulation device. The disclosed transcutaneous spinal cord stimulation methods may include applying stimulation patterns via the multiple electrodes that include an initial gradual increase in stimulation amplitude of all active electrodes to a baseline stimulation amplitude, and specific electrode activation from the baseline stimulation amplitude to an effective stimulation amplitude at the appropriate movement phase.

Transcutaneous spinal cord stimulation may be achieved through a small-diameter multielectrode configuration around the T10-T11 vertebral segment. In other aspects, the preferential activation of specific muscle groups is determined by calculating the selectivity index and recruitment probability. In another aspect, the specific muscle groups targeted may be in the hip, legs, and ankles. In yet another aspect, altering the position of the electrodes may provide unique degrees of selectivity for different muscles.

All the electrodes may be ramped up to a designated baseline stimulation amplitude. In another aspect, the electrode may ramp-up from baseline to an effective stimulation amplitude, and then return to baseline. In an additional aspect, the ramp-up from baseline to an effective stimulation amplitude may take as little as 0.01 seconds.

The electrodes of the multielectrode-configured device may be activated singularly or in combination with any other electrode(s). In other aspects, the electrodes may be activated in sequence. In yet another aspect, the electrodes may be positioned to stimulate specific muscle groups.

In one aspect, a method of transcutaneous spinal cord stimulation of a subject is disclosed that includes providing a multielectrode stimulation device comprising at least two transdermal spinal cord stimulation (tSCS) electrodes and at least one return electrode; positioning a first and second tSCS electrode of the at least two tSCS electrodes on the back of the subject at a symmetrical lateral separation distance from a midline of the patient and at a shared rostrocaudal position of the patient, respectively, wherein the positioning is selected to activate at least one motor neuron pool; positioning the at least one return electrode on an abdomen of the subject; and activating the at least one specific motor neuron pool to stimulate at least one specific muscle group using a predetermined stimulation pattern, the predetermined stimulation pattern comprising a temporal schedule of at least one of a stimulation voltage or a stimulation amperage for each of the at least two tSCS electrodes. In some aspects, the predetermined stimulation pattern further includes at least one of a stimulation frequency and a stimulation amplitude. In some aspects, the temporal schedule of stimulation voltages or amperages comprises increasing the stimulation amplitude in all the tSCS electrodes at a baseline rate to a baseline stimulation amplitude; maintaining all the tSCS electrodes at the baseline stimulation amplitude; and for at least one of the tSCS electrodes, increasing the stimulation amplitude at an activation rate from the baseline stimulation amplitude to an activation amplitude, maintaining the activation amplitude for a stimulation period, and decreasing the stimulation amplitude back to the baseline stimulation amplitude at a deactivation rate; wherein the baseline rate is slower than either the activation rate or the deactivation rate. In some aspects, the baseline stimulation amplitude ranges from about 50% to about 95% of the motor threshold amplitude, wherein the motor threshold amplitude comprises the stimulation amplitude that induces a peak-to-peak response amplitude of at least about 20 μV within a latency ranging from about 10 to about 30 ms in any muscle. In some aspects, the baseline rate comprises increasing the stimulation amplitude from zero to the baseline stimulation amplitude over a habituation period ranging from about ten seconds to about ten minutes; the activation rate comprises increasing the stimulation amplitude from the baseline stimulation amplitude to the activation amplitude over an activation period ranging from instantaneous to about 1 minute; and the deactivation rate comprises decreasing the stimulation amplitude from the activation amplitude to the baseline stimulation amplitude over a deactivation period ranging from instantaneous to about 1 minute. In some aspects, the activation amplitude ranges from the motor threshold amplitude to a saturation amplitude, wherein the saturation amplitude comprises: a stimulation amplitude limit at which no additional increase in a response amplitude of the first recruited muscles is observed; or a maximum stimulation amplitude tolerated by the subject. In some aspects, the at least two electrodes may be activated singularly or in combination with any of the other electrodes. In some aspects, the at least two electrodes are activated sequentially. In some aspects, the predetermined stimulation pattern comprises the stimulation frequency selected to selectively target a flexor or extensor muscle group, wherein the stimulation frequency ranging from about 20 Hz to about 40 Hz selectively targets the extensor muscle group; and the stimulation frequency ranging from about 40 Hz to about 100 Hz selectively targets the flexor muscle group; and the activation amplitude selected to recruit a portion of the flexor or extensor muscle group. In some aspects, wherein the at least two tSCS electrodes of the multielectrode stimulation device are positioned at a vertebral segment selected from a thoracic or lumbar vertebral segment for assisting lower limb movements; or a cervical vertebral segment for assisting upper limb movements. In some aspects, the thoracic or lumbar vertebral segment is selected from a vertebral segment ranging from T9 to L1; and the cervical vertebral segment is selected from a vertebral segment ranging from C3 to T1. In some aspects, the activation of a specific muscle group is determined using a selectivity index (SI) and a recruitment probability, wherein the selectivity index SI of muscle m is a difference between a normalized recruitment of muscle m (RECm) and an average normalized recruitment of M−1 muscles, excluding the muscle, m (RECn), as expressed by:

$${SI}_{m} = {REC}_{m} - \frac{{\sum }_{n \neq m}^{M} {REC}_{n}}{M - 1}$$

the recruitment probability estimates an activation of motoneuron pools in each spinal segment (Si) as a linear combination of each normalized muscle recruitment Mj and Wij, an expected segmental distribution of motoneuron pools innervating muscle j at spinal segment Si, as expressed by:

$${SI}_{m} = {REC}_{m} - \frac{{\sum }_{n \neq m}^{M} {REC}_{n}}{M - 1} .$$

In some aspects, the specific muscle groups targeted are selected from hip muscle groups, leg muscle groups, ankle muscle groups, shoulder muscle groups, arm muscle groups, forearm muscle groups, hand muscle groups, and any combination thereof. In some aspects, the method further includes altering the placement of the at least two transdermal spinal cord stimulation (tSCS) electrodes to provide selectivity for different muscle groups.

In another aspect, a non-invasive brain-spine interface (BSI) system to facilitate movements in a subject with a spinal cord injury (SCI). The system includes a wearable electroencephalogram EEG device comprising an array of surface EEG electrodes configured to detect a plurality of signals indicative of brain activity in the brain of the subject; a multielectrode transcutaneous spinal cord stimulation (tSCS) device comprising an array of tSCS electrodes configured for placement over a predetermined region of the subject's back and a pair of return electrodes positioned over a left and right region of the subject's abdomen, wherein the predetermined region is selected to activate at least one motor neuron pool that stimulates at least one specific muscle group; and a computing device operatively coupled to the wearable EEG device and the tSCS device. The computing device comprising at least one processor configured to receive the plurality of signals from the wearable EEG device; identify a movement intention by the subject based on the plurality of signals using a machine learning model and producing a movement intention signal; and activate the tSCS device to produce stimulations using a predetermined stimulation pattern, the predetermined stimulation pattern comprising a temporal schedule of at least one of a stimulation voltage or a stimulation amperage for each of the at least two tSCS electrodes, predetermined stimulation pattern further comprising a predetermined series of voltages or amperages delivered to the subject through individual stimulation electrodes from the array of stimulation electrodes, the stimulation pattern configured to stimulate muscle activation patterns, wherein the muscle activation patterns are configured to facilitate movements of the subject. In some aspects, the wearable electroencephalogram EEG device comprises an array of 32 surface EEG electrodes. In some aspects, the array of surface EEG electrodes is configured to be positioned over a sensorimotor cortex of the subject. In some aspects, the machine learning model comprises a linear discriminant analysis (LDA) classifier model. In some aspects, the at least one processor is further configured to train the machine learning model using a training dataset comprising a plurality of EEG signal sets recording using the EEG device, wherein each EEG signal set is associated with an intended movement of the subject. In some aspects, the predetermined stimulation pattern further includes at least one of a stimulation frequency and a stimulation amplitude, wherein: the stimulation frequency is selected to selectively target a flexor or extensor muscle group, wherein, the stimulation frequency ranging from about 20 Hz to about 40 Hz selectively targets the extensor muscle group and the stimulation frequency ranging from about 40 Hz to about 100 Hz selectively targets the flexor muscle group; and the stimulation amplitude ranges from a motor threshold amplitude to a saturation amplitude, wherein: the motor threshold amplitude comprises the stimulation amplitude that induces a peak-to-peak response amplitude of at least about 20 μV within a latency ranging from about 10 to about 30 ms in any muscle; and the saturation amplitude comprises a stimulation amplitude limit at which no additional increase in a response amplitude of the first recruited muscles is observed; or a maximum stimulation amplitude tolerated by the subject. In some aspects, the predetermined stimulation pattern further includes a habituation to a baseline stimulation amplitude, wherein the baseline stimulation amplitude ranges from about 50% to about 95% of the motor threshold amplitude.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A contains images depicting the experimental framework to compare muscle recruitment selectivity by conventional (left, top) and multielectrode tSCS (left, bottom) systems. The rostrocaudal and unilateral organization of leg muscles' motor neuron pools in the spinal cord enables the selective recruitment of individual muscle groups by small diameter electrodes centered at the T11/T12 vertebrae. RF: rectus femoris; VL: vastus lateralis; ST: semitendinosus; TA: tibialis anterior; MG: medial gastrocnemius; SL: soleus.

FIG. 1B is an image modeling the stimulation and return electrode placement, and the leg muscle responses recorded while pulses of tSCS were delivered at increasing stimulation amplitudes.

FIG. 1C is a graph of stimulation amplitude (left) and response amplitude (right) quantifying each muscle's recruitment curve. A peak-to-peak response amplitude was computed across a range of increasing stimulation amplitudes. Scale bars: 100 μV and 10 ms.

FIG. 1D is a schematic that depicts non-invasive transcutaneous spinal cord stimulation (left) and enables non-selective activation of leg muscle groups (right) below a spinal cord injury. Muscle groups: RF: rectus femoris; VL: vastus lateralis; ST: semitendinosus; TA: tibialis anterior; MG: medial gastrocnemius; SL: soleus

FIG. 1E is a graph showing continuous stimulation of muscle groups regardless of movement phase during five hip flexion attempts in participants with SCI. RF: rectus femoris; VL: vastus lateralis; ST: semitendinosus; TA: tibialis anterior; MG: medial gastrocnemius; SL: soleus

FIG. 1F is a schematic that depicts epidural spinal cord stimulation to enable stimulations delivered at three cathode locations (left) in a spatiotemporal pattern to activate specific muscle groups (right) in a sequence and timing consistent with walking.

FIG. 1G is a graph of EMGs of muscle groups activated at the appropriate movement phase using epidural spinal cord stimulation in the spatiotemporal pattern illustrated in FIG. 1F. Gray bars represent the stance phase. IL: iliacus; RF: rectus femoris; VL: vastus lateralis; ST: semitendinosus; TA: tibialis anterior; SL: soleus

FIG. 1H is a set of images depicting the multielectrode array and the corresponding target muscles. A small diameter electrode positioned rostrally and over the right side would enable the selective targeting of right leg proximal muscles (hip, thigh), while a right caudal electrode would target right distal muscles (ankle). RF: rectus femoris; VL: vastus lateralis; ST: semitendinosus; TA: tibialis anterior; MG: medial gastrocnemius; SL: soleus

FIG. 1I is a schematic that depicts multielectrode transcutaneous spinal cord stimulation. Electrodes targeting muscle groups in the hip (blue), knee (light blue), and ankle (green) are activated at the appropriate movement phases.

FIG. 1J is a graph of effective stimulation amplitude, depicting a slow ramp-up of the voltage in all electrodes and then a shorter ramp-up of stimulations to spatially selective individual electrodes.

FIG. 2A is a set of images and graphs depicting muscle recruitment and selectivity by conventional tSCS. Overlaid EMG responses are shown over a broad range of SCS amplitudes for the conventional electrode in a representative participant. Stimulation artifacts have been blanked for illustration purposes.

FIG. 2B is a set of images and graphs depicting muscle recruitment and selectivity by multielectrode tSCS. Overlaid EMG responses for the right caudal electrode in the same participant.

FIG. 2C is a graph summarizing muscle recruitment of various muscle groups in response to single pulse stimulation by conventional tSCS in a representative participant. Recruitment was averaged across n=4 repetitions for each stimulation amplitude. The peak-to-peak amplitude was calculated to create a recruitment curve for each recorded leg muscle (color traces: targeted muscles). RF: rectus femoris; VL: vastus lateralis; ST: semitendinosus; TA: tibialis anterior; MG: medial gastrocnemius; SL: soleus; R: right leg; L: left leg.

FIG. 2D is a graph of recruitment curves for muscle responses to stimulation by the right caudal electrode of a multielectrode tSCS array. Recruitment was averaged across n=4 repetitions for each stimulation amplitude. The peak-to-peak amplitude was calculated to create a recruitment curve for each recorded leg muscle (color traces: targeted muscles). RF: rectus femoris; VL: vastus lateralis; ST: semitendinosus; TA: tibialis anterior; MG: medial gastrocnemius; SL: soleus; R: right leg; L: left leg.

FIG. 2E is a set of graphs illustrating normalized muscle recruitment (top), selectivity index computation (middle), and selectivity index (bottom). The selectivity index computation is the relative recruitment of a given muscle compared to all other muscles.

FIG. 2F is a set of graphs of the selectivity index for all recorded muscles in conventional (top) and right caudal (bottom) electrode tSCS in a subject. RF: rectus femoris; VL: vastus lateralis; ST: semitendinosus; TA: tibialis anterior; MG: medial gastrocnemius; SL: soleus; R: right leg; L: left leg.

FIG. 3A is a schematic and corresponding graphs of selectivity index comparison between rostrocaudal positions for ipsilateral electrodes. Selectivity index comparison between electrodes in three rostrocaudal positions ipsilateral to the targeted muscle. The electrode with the highest median selectivity index is illustrated in color and was chosen as the target electrode for that muscle. Muscles from both legs are pooled together, with two data points per participant (e.g. one data point for the right RF targeted by the right rostral electrode and one data point for the left RF targeted by the left rostral electrode). RF: rectus femoris; VL: vastus lateralis; ST: semitendinosus; TA: tibialis anterior; MG: medial gastrocnemius; SL: soleus. The central white circles with colored outlines are the median. Friedman test for repeated measures for the effect of electrode position on selectivity index followed. Wilcoxon signed rank test with Bonferroni correction for multiple comparisons for comparisons across electrode positions. *p<0.05; **p<0.01; ***p<0.001.

FIG. 3B is a schematic and set of corresponding graphs of selectivity index comparison between conventional tSCS electrode and target electrode. RF: rectus femoris; VL: vastus lateralis; ST: semitendinosus; TA: tibialis anterior; MG: medial gastrocnemius; SL: soleus. The central white circles with colored outlines are the median. Friedman test for repeated measures for the effect of electrode position on selectivity index followed. Wilcoxon signed rank test with Bonferroni correction for multiple comparisons for comparisons across electrode positions. *p<0.05; **p<0.01; ***p<0.001.

FIG. 3C is a schematic and set of corresponding graphs of selectivity index comparison between the target ipsilateral electrode and its contralateral counterpart. Selectivity indices for most muscles were higher for electrodes ipsilateral to the targeted muscle than those achieved by the contralateral electrode. RF: rectus femoris; VL: vastus lateralis; ST: semitendinosus; TA: tibialis anterior; MG: medial gastrocnemius; SL: soleus. The central white circles with colored outlines are the median. Friedman test for repeated measures for the effect of electrode position on selectivity index followed. Wilcoxon signed rank test with Bonferroni correction for multiple comparisons for comparisons across electrode positions. *p<0.05; **p<0.01; ***p≤0.001.

FIG. 4A is a schematic of paired-pulse response in SL muscle. The small-diameter electrodes in the multielectrode configuration were verified to recruit primarily afferent fibers by the suppression of posterior-root muscle reflexes using a paired-pulse paradigm with an inter-pulse interval of 33.3 ms. The amount of suppression was quantified by the response amplitude ratio between the responses to the first and the second stimulation pulses.

FIG. 4B is an image depicting the activation of the left caudal electrode and a set of corresponding graphs. Recruitment curves in a representative participant for the first (top) and second (bottom) pulse responses in the left caudal electrode from the multielectrode configuration.

FIG. 4C a set of graphs of normalized responses to first and second peak-to-peak responses at the highest stimulation amplitude in a subject. Peak responses are normalized to the highest muscle response across the multielectrode configuration. bars represent the mean±s.d. Paired-sample t-test.

FIG. 4D is a set of graphs of the R2−R1 amplitude ratio across participants and electrodes. The right and left legs are pooled together. A value of 1 would indicate that the first and second responses had equal magnitudes, whereas a value <1 would indicate that the response to the second stimulation pulse was lower than the one to the first pulse. The colored circle in the array indicates the optimal electrode for each muscle. the central circle is the median, while the top and bottom edges represent the 75th to 25th percentile, respectively. One-sample Wilcoxon signed rank test with the alternative hypothesis that μ−1<0. *p<0.05; **p<0.01; ***p<0.001.

FIG. 5A is a set of graphs of muscle recruitment probability for each muscle when the active electrode is at different rostrocaudal levels ipsilaterally (color) and contralaterally (grey) to the recorded muscle. Data were derived from the probability of recruiting a given muscle at a specific electrode position across all participants, where a probability of 1 would indicate that that muscle was maximally recruited for all participants at that electrode position. Note that values for recruitment probability by contralateral electrodes are also positive.

FIG. 5B is a schematic of segmental innervation probabilities for recorded leg muscles at different spinal segments. Innervation probabilities are reflected by percentage and opacity.

FIG. 5C is a set of images depicting the computation of spinal activation maps as a linear combination of the normalized recruitment of each muscle and its innervation probability at each segment.

FIG. 5D is a set of images and corresponding graphs of recruitment probabilities and motor neuron activation maps enabled by each electrode configuration.

FIG. 6 is a schematic of muscle electromyography (EMG) activity and transcutaneous spinal cord stimulation amplitude during training right leg extension trial in a representative participant. A stimulation paradigm was implemented to ensure tolerable jumps in stimulation amplitude at the appropriate phases of movement during the trial. Stimulation amplitude was increased from 0 to the rest stimulation amplitude (10 mA) during the first 7 seconds of the trial. Stimulation amplitude was then remotely controlled and timed to movement instructions. Stimulation was alternated between the rest (10 mA) and movement (15 mA) intensities during the movement cues.

FIG. 7A is a schematic of the technological and experimental framework for a non-invasive brain-spine interface.

FIG. 7B is a set of schematics and plots that show leg extension predicted from event-related desynchronization that is used to enable 30 Hz tSCS.

FIG. 8A is a set of schematics and plots representing the extracting of spectral, temporal, and spatial features from EEG data described in the current disclosure.

FIG. 8B is a set of plots showing the testing of the leg extension onset decoder on offline data.

FIG. 9A is a set of plots showing how the neural decoder is trained on cued overt movements tested on cued imaginary “movement”.

FIG. 9B is a set of plots showing how the neural decoder is trained on cued overt movements tested on uncued “free” movements.

FIG. 10A is a set of schematics and plots showing how multielectrode tSCS enables targeted right hip activation.

FIG. 10B is a set of plots showing the decoder trained on cued movements with stimulation and tested on uncued “free” movements with stimulation.

FIG. 11 is a set of brain maps showing that event-related desynchronization in the leg sensorimotor cortex enables consistent decoder performance across conditions. Group analysis reveals differences in desynchronization strategies across tasks.

FIG. 12A is a spinal activation map and recruitment probability for leg muscles stimulated with a left rostral electrode in 16 participants.

FIG. 12B is a set of plots of selectivity index comparison between a conventional tSCS electrode (conv.) and the target electrode (target) for recruitment of ankle muscles. Muscles from both legs are pooled together, with two data points per participant (e.g., one data point for the right TA targeted by the right caudal electrode and one data point for the left TA targeted by the left caudal electrode). RF: rectus femoris; VL: vastus lateralis; ST: semitendinosus; TA: tibialis anterior; MG: medial gastrocnemius; SL: soleus.

FIG. 13A is a photo and pair of plots showing exemplary movements for two participants with SCI attempting to move the right hip (S01) and the left knee (S02) without tSCS. Muscles from the leg attempting the movement are shown in color. Kinematic tracking for S01 was not possible due to poor lighting conditions. RF: rectus femoris; VL: vastus lateralis; ST: semitendinosus; TA: tibialis anterior; MG: medial gastrocnemius; SL: soleus.

FIG. 13B is a photo and pair of plots showing exemplary movements for two participants with SCI attempting to move the right hip (S01) and the left knee (S02) with conventional tSCS. Muscles from the leg attempting the movement are shown in color. Kinematic tracking for S01 was not possible due to poor lighting conditions. RF: rectus femoris; VL: vastus lateralis; ST: semitendinosus; TA: tibialis anterior; MG: medial gastrocnemius; SL: soleus.

FIG. 13C is a photo and pair of plots showing exemplary movements for two participants with SCI attempting to move the right hip (S01) and the left knee (S02) with multielectrode tSCS. Muscles from the leg attempting the movement are shown in color. Kinematic tracking for S01 was not possible due to poor lighting conditions. RF: rectus femoris; VL: vastus lateralis; ST: semitendinosus; TA: tibialis anterior; MG: medial gastrocnemius; SL: soleus.

FIG. 14A is a schematic showing the effect of spatially selective stimulation on functional movements.

FIG. 14B is a schematic showing the effect of stimulation frequency on flexion vs. extension movements.

FIG. 14C is an image of an RT300 leg cycle used in a pilot clinical trial of the SCS stimulation protocol disclosed herein.

FIG. 15A is a plot that shows that SCS enables force production in paralyzed muscles. Individuals with SCI attempt to produce torque with their hip muscles without and with epidural spinal cord stimulation.

FIG. 15B is another plot that shows that SCS enables force production in paralyzed muscles. Individuals with SCI attempt to produce torque with their hip muscles without and with epidural spinal cord stimulation.

FIG. 16 is a plot showing the expected range of recruitment over a range of effective stimulation amplitudes. Stimulation amplitude to facilitate movement at a given joint is expected to fall within a range where the target muscles are active while the likelihood of activating non-targeted muscles is reduced. MT: motor threshold; RF: rectus femoris; VL: vastus lateralis; ST: semitendinosus; TA: tibialis anterior; MG: medial gastrocnemius; SL: soleus; R: right leg; L: left leg.

FIG. 17 is a schematic of a pre-programmed stimulation protocol for ergometer cycling. The ONWARD ARC-Ex platform allows the alternation of stimulation protocols with alternating channels and stimulation frequencies. A touch-based tablet allows a trained therapist to intuitively modify stimulation parameters.

FIG. 18 is a table assessing the components and their functionality of the systems and methods of the present disclosure.

FIG. 19 is a table showing functional improvements after long-term training with SCS and activity-based training. Summary of recovery outcomes as a function of lesion severity assessed by AIS classification at study enrollment. Transparent circles indicate not all participants with that AIS classification achieved the outcome. Adjacent ratios indicate the partial number.

FIG. 20A is a schematic of a paired-pulse stimulation paradigm to evaluate the amount of post-activation depression by quantifying the suppression of the response to the second stimulation pulse. Reflex-mediated responses would result in a significant amount of suppression, whereas no suppression would be observed in the recruitment of efferent fibers bypassing the synapse.

FIG. 20B is a schematic that shows the evaluation of a 2 ms conventional waveform and a 1 ms KHF waveform.

FIG. 20C is a graph of the amount of post-activation depression across muscles in lumbar tSCS. Abbreviations: Kilohertz frequency (KHF), adductor magnus (AM), rectus femoris (RF), biceps femoris (BF), semitendinosus (ST), vastus lateralis (VL), tibialis anterior (TA), medial gastrocnemius (MG), and soleus (SOL), suppression (supp.). N=10.

FIG. 21 is a graph of motor thresholds across N=59 unimpaired participants. MT: motor threshold; RF: rectus femoris; VL: vastus lateralis; ST: semitendinosus; TA: tibialis anterior; MG: medial gastrocnemius; SL: soleus; R: right leg; L: left leg.

FIG. 22 is a graph of the range of motion with different types of tSCS. Right knee extension with conventional and multielectrode tSCS compared to no stimulation in participant S002 (from FIG. 13, participant with smallest degree of improvement).

FIG. 23 is a graph of frequency-modulation of flexor and extensor muscles in eSCS. Mean (+s.e.m.) modulation of EMG amplitude in flexor and extensor muscles during walking for representative participant with SCI.

FIG. 24A is a reading of vastus lateralis activity during continuous tSCS at motor threshold over stimulation frequencies.

FIG. 24B is a schematic of overlaid responses colored according to their respective segments in FIG. 24A. Note the appearance of early latency responses at frequencies between 20-50 Hz.

FIG. 24C is a schematic of hypothesized pathways for mediation of early (monosynaptic), medium (di-synaptic), and late (polysynaptic) latency responses observed in FIG. 24B.

FIG. 24D is a set of graphs of effect of tSCS frequency on torque generation for flexion and extension of the knee and ankle in one participant with SCI (AIS-D). Note the opposite trends with frequency between flexion and extension at the knee, while low frequencies generate larger extension torques at the ankle.

FIG. 25 is a schematic of stimulation intensity in targeted tSCS alternates with task requirements. Stimulation intensity of the right, T11/T12 electrode is gradually increased at the onset of training and alternates with right knee extension. Data from N=9 unimpaired participants. EMGs normalized to their respective maximum amplitudes. RF: rectus femoris; VL: vastus lateralis; TA: tibialis anterior; MG: medial gastrocnemius.

FIG. 26A is a graph of stimulation protocol that increased amplitude from x % to 100% of motor threshold using different magnitudes.

FIG. 26B is a graph of a stimulation protocol that gradually increased from 50%-100% of motor threshold using different speeds (15 sec-1 min).

FIG. 27A is a schematic of post-activation depression by single-pulse stimulation at 100% and 250% of motor threshold. Note that the degree of post-activation decreases for some leg muscles as stimulation amplitude increases, suggesting higher recruitment of efferent fibers. N=10.

FIG. 27B is a set of graphs of continuous 30 Hz tSCS at different stimulation amplitudes elicit responses even at amplitudes below motor threshold (80% MT). Moreover, evoked responses for different stimulation amplitudes have different shapes and latencies.

FIG. 27C is a schematic of hypothesized neural circuits mediating responses of different latencies in FIG. 27B. While the medium and early latency responses are hypothesized to be reflex-mediated, the extremely short latency (˜7 ms) of responses at 120% MT suggests the direct recruitment of efferent motor fibers. Abbreviations: adductor magnus (AM), rectus femoris (RF), biceps femoris (BF), semitendinosus (ST), vastus lateralis (VL), tibialis anterior (TA), medial gastrocnemius (MG), and soleus (SL).

FIG. 28 is a block diagram schematically illustrating a system in accordance with one aspect of the disclosure.

FIG. 29 is a block diagram schematically illustrating a computing device in accordance with one aspect of the disclosure.

FIG. 30 is a block diagram schematically illustrating a remote or user computing device in accordance with one aspect of the disclosure.

FIG. 31 is a block diagram schematically illustrating a server system in accordance with one aspect of the disclosure.

### DETAILED DESCRIPTION OF THE INVENTION

Transcutaneous spinal cord stimulation (tSCS) has been gaining momentum as a non-invasive approach to restore movement to paralyzed muscles in spinal cord injury (SCI). However, stimulation is typically delivered continuously and controlled by experimenters, regardless of movement intention. As described in the examples herein, brain-controlled stimulation results in improved functional outcomes compared to continuous or sham stimulation in stroke patients.

Spinal cord stimulation (SCS) is a neuromodulation intervention for subjects with spinal cord injuries to restore movement to paralyzed areas below the injury. By selectively activating individual muscle groups at the appropriate phases of movement, epidural SCS (eSCS) can re-enable the performance of dexterous activities, however, this is an invasive and prohibitively expensive prohibited procedure. Transcutaneous SCS (tSCS) offers a promising non-invasive alternative, however, conventional applications of tSCS result in non-selective recruitment of all the muscles innervated in that area.

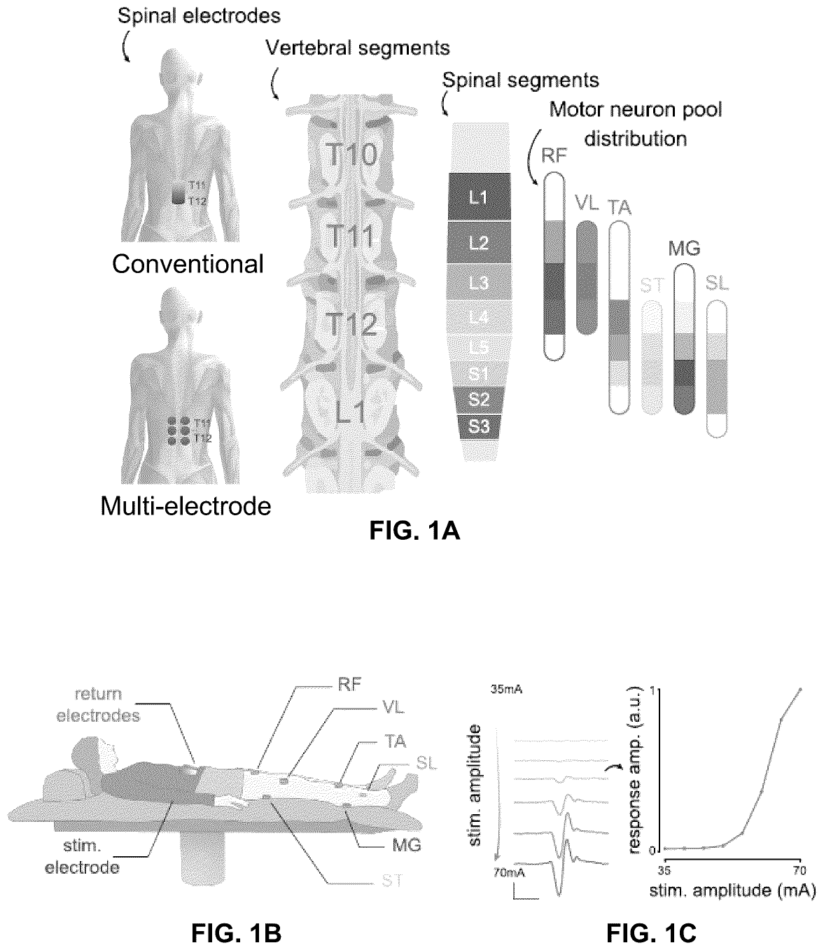

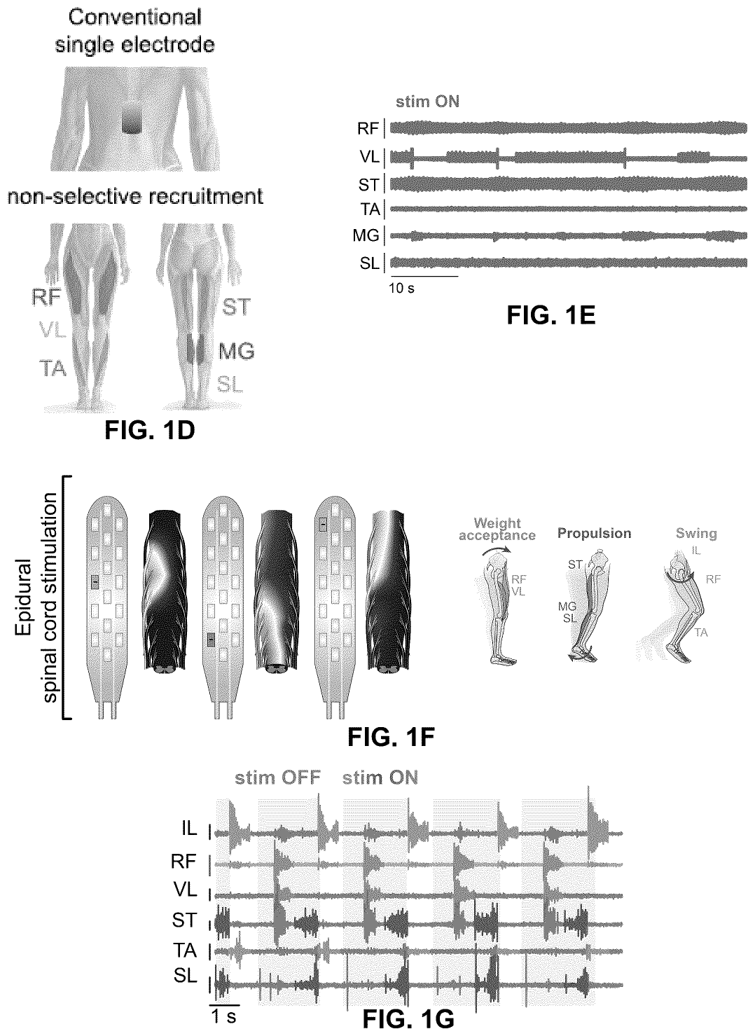

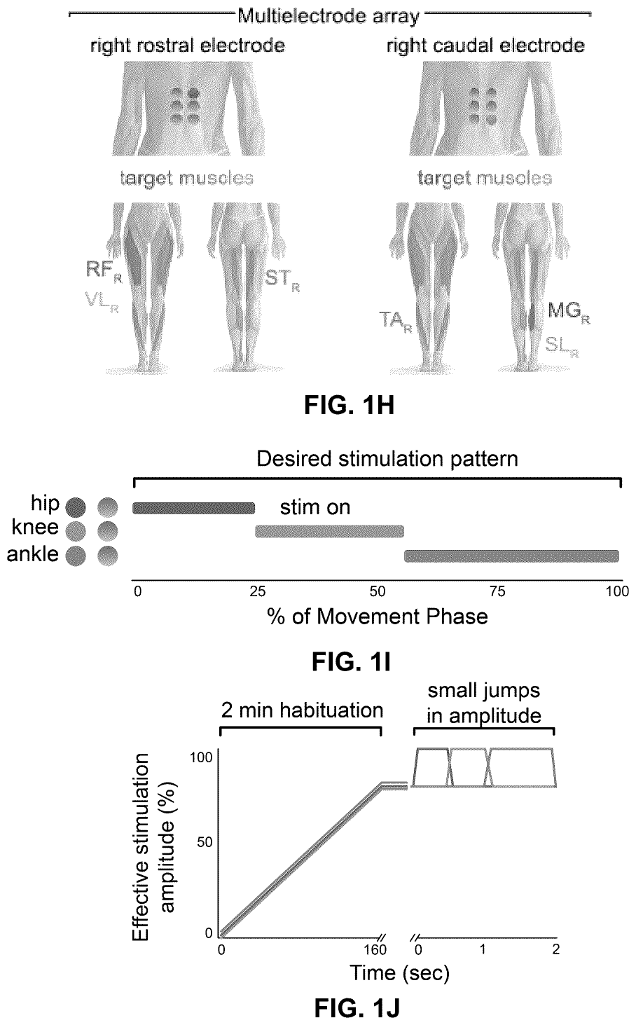

In various aspects, the transcutaneous spinal cord stimulation is delivered to the subject using a multielectrode array, including, but not limited to, a six-electrode array as illustrated in FIG. 1H. In various aspects, the multielectrode array is positioned over the spinal cord at locations suitable to preferentially target specific muscle groups (FIG. 1H). The timing and amplitudes of stimulations may be switched between individual electrodes of the multielectrode array to selectively recruit muscle activity in muscle groups appropriate to facilitate a movement including, but not limited to, walking, as illustrated in FIG. 1I.

A current limitation in the field of tSCS is that high currents are needed to recruit both leg and arm muscles. Turning the stimulation from off to on is not feasible, as people feel pain and discomfort from this sudden and drastic change in stimulation amplitude. This discomfort is compounded by the continuous switching stimulation between individual electrodes of the multielectrode array to facilitate movements of the subject such as walking.

In various aspects, the stimulation amplitudes of all electrodes in the multielectrode array are increased gradually to a baseline stimulation level that is slightly below the level needed to recruit activation of muscle groups, but that reduces the level of increased stimulation needed to selectively recruit muscle activity as described above, as illustrated in FIG. 1J. Without being limited to any particular theory, the reduced increase in amplitude changes is associated with less discomfort for subjects using the tSCS devices, systems, and methods as described herein for therapy. In various aspects, all electrodes in the multielectrode array are maintained at the baseline stimulation level throughout therapy. Each electrode increases the stimulation level from the baseline stimulation level to an activation level during an appropriate phase of a desired movement and then returns to the baseline stimulation level once the appropriate phase of the desired movement is completed.

In various aspects, the disclosed devices, systems, and methods make use of the stimulation paradigm described above that enables the alternation of stimulation delivered amongst an array of at least two active electrodes without causing discomfort to the subject. In some aspects, this stimulation paradigm is characterized by an initial gradual increase of all electrodes in the array over a habituation period up to a baseline stimulation amplitude, defined herein as a selected percentage of the motor threshold amplitude at which muscle groups are minimally responsive, as illustrated in FIG. 1J. In some aspects, to activate each electrode of the array at an appropriate movement phase, the stimulation amplitude for a given electrode increases from the baseline stimulation amplitude to an activation amplitude at which one or more muscle groups are responsive.

By way of non-limiting example, the stimulation amplitude of a selected electrode of the multielectrode array is increased to 100% of the motor threshold amplitude during an appropriate movement phase, with a significantly shorter (˜0.01 sec) ramp-up time (FIG. 1J). In some aspects, the range of activation amplitude as described above may fall within a range tailored to an individual subject ranging between (1) a minimum motor threshold amplitude at which a peak-to-peak response amplitude of at least 20 μV is observed within a latency of 10-30 ms in any muscle, and (2) the saturation amplitude at which there is no longer an increase in response amplitude of the first recruited muscles or the maximum amplitude tolerated by a subject. The potential changes in stimulation amplitude between the baseline stimulation amplitude and the range of activation amplitudes defined above is considerably less than existing changes in stimulation amplitude delivered by stimulation electrodes maintained at a baseline stimulation amplitude of zero when not actively stimulating muscle groups. The lower increase to activation amplitudes associated with the disclosed tSCS systems, devices, and methods results in a tSCS therapy that is better tolerated by subjects.

In various aspects, the baseline stimulation amplitude may range from about 50% to about 95% of the motor threshold amplitude described above. In various additional aspects, the habituation period over which the initial gradual increase of all electrodes in the array up to a baseline stimulation amplitude may range from about ten seconds to about ten minutes. In various other aspects, the activation rate at which the stimulation amplitude is increased from the baseline stimulation amplitude to the activation amplitude may range from an essentially instantaneous step function to about 1 minute. In various other additional aspects, the deactivation rate at which the stimulation amplitude is decreased from the activation amplitude back to the baseline stimulation amplitude may range from an essentially instantaneous step function to about 1 minute.

Disclosed herein is a technological framework to evaluate and quantify muscle recruitment selectivity enabled by different electrode configurations of tSCS. The optimal cathode positions to target different leg muscles are identified by computing the selectivity index across 12 muscles and electrode configurations in 16 participants, with a total of 192 analyzed muscles. A small-diameter multielectrode configuration of tSCS over the T10-L1 vertebral segments can enhance the rostrocaudal and unilateral recruitment selectivity of leg muscles compared to conventional tSCS using a single large electrode over the T11-T12 vertebra. Verification of post-activation depression suggests that these improvements in muscle recruitment selectivity are mediated by posterior root-muscle reflexes, which is a promising finding for the translation of this technology into clinical practice, with implications in neurorehabilitation for people with neuromotor disorders.

Additionally, a current limitation in the field of tSCS is that high currents are needed to recruit both leg and arm muscles. Turning the stimulation from off to on is not feasible, as people feel pain and discomfort from this sudden and drastic change in amplitude. The current approach is therefore to gradually increase the stimulation amplitude (in about 2 minutes) from 0 milliamps (mA) to the stimulation amplitude that will be used for therapy (effective amplitude, say 100 mA). Then, this stimulation amplitude is left at that level throughout therapy.

Stimulation amplitude, as used herein, refers to the magnitude of the electrical stimulation applied via the stimulation electrodes, including, but not limited to, voltage, amperage, power, and any other expression of electrical stimulation amplitude without limitation.

The disclosed invention consists of a stimulation paradigm that would enable the alternation between active electrodes without causing discomfort. The systems and methods disclosed herein are configured to implement an initial gradual increase in stimulation amplitude, as in the current approach, which increases to a baseline stimulation amplitude characterized as a predetermined percentage of the effective stimulation amplitude, including, but not limited to from about 50% to about 95% of the effective stimulation amplitude. This initial increase occurs in all electrodes prior to activating any electrode. To activate each electrode at the appropriate movement phase, the stimulation amplitude for a given electrode increases from the baseline stimulation amplitude to at least 100% of the effective stimulation amplitude in that movement phase, with a significantly shorter (˜0.01 sec) ramp-up. Without being limited to any particular theory, it is thought that maintaining all inactive electrodes at a baseline stimulation amplitude provides for smaller relative increases in stimulation amplitude when an electrode is activated, and this smaller relative increase in stimulation amplitude is more tolerable to the stimulated subject as compared to a stimulation with a larger relative increase from zero stimulation to the activated stimulation amplitude.

This disclosure demonstrates that it is possible to have a single experimental setup in a multielectrode configuration that enables the combination of independent observations of improvements in the selectivity of proximal vs. distal as well as unilateral muscles. This study aimed to exploit the organization of motor neuron pools in the spinal cord to identify stimulation sites that result in optimal recruitment selectivity for different key leg muscles. Evoked compound muscle action potentials of lower-limb muscles in response to stimulation over the T10-L1 vertebral segments using conventional and multielectrode configurations of tSCS were studied (FIG. 1A, FIG. 1B). First, recruitment curves for individual leg muscles (FIG. 1C) were analyzed when the cathode was a single large electrode as in conventional tSCS (centered at T11/T12) or in one of six locations in a multielectrode configuration. The recruitment selectivity enabled for each muscle at each cathode location was compared and optimal electrode configurations that enabled the highest selectivity for that muscle were identified.

Based on the rostrocaudal anatomical distribution of the motor neuron pools that innervate leg muscles (FIG. 1A), it was hypothesized that while conventional tSCS results in the non-selective recruitment of proximal and distal leg muscles (FIG. 1D, FIG. 1E), the multielectrode configuration would enable preferential mediolateral and rostrocaudal recruitment of leg muscles. For example, a right-side cathode centered rostrocaudally at the T10/T11 interspinous ligament (overlapping the L1-L3 spinal segments) would primarily recruit proximal muscles in the right leg, whereas a right-side cathode centered at the T12/L1 interspinous ligament (overlapping the L4-S3 spinal segments) would primarily recruit the right distal muscles (FIG. 1H, FIG. 1I, FIG. 1J).

Innovations in electrode positioning and multi-site stimulation have recently gained interest in the field of tSCS. However, various studies have demonstrated that changes in body position, as well as the placement of anode and cathode electrodes, can significantly increase the likelihood of recruiting motor efferent fibers directly. Presynaptic recruitment at the posterior root afferents is preferred for neurorehabilitation, as this mechanism allows for spared spinal circuits and supraspinal inputs to interact with the stimulation. Therefore, to facilitate the translation of our approach into a clinical setting and minimize the possibility of habituation effects resulting from continuous stimulation of efferent fibers, it is beneficial to conduct a comprehensive investigation into the activation mechanisms that underlie the observed improvements in muscle recruitment selectivity. The improved selectivity achieved through the multielectrode configuration would be mediated by the activation of muscle recruitment via sensory afferents, as evidenced by post-activation depression of the evoked response.

Finally, the recruitment probability of a given muscle at a specific cathode site was combined with previously reported segmental innervation probabilities at different spinal segments to validate the results and provide a neuroanatomical map of recruited spinal segments by each electrode configuration. Gaining a better understanding of the neural mechanisms behind these improvements in muscle recruitment selectivity by spatially selective tSCS may expedite the development of non-invasive technologies that can re-enable a broad repertoire of dexterous movements in rehabilitation and daily life.

Previous research in non-invasive tSCS has shown that stimulating different areas of the lumbar spinal cord can selectively activate motor neuron pools in leg muscles. Specifically, rostral and caudal positions of small electrodes centered over the midline have been shown to activate proximal and distal muscle groups, respectively, and lateral positions can selectively recruit both proximal and distal muscles ipsilateral to the cathode position.

This disclosure demonstrates that by combining these approaches, selective activation of specific muscle groups in the hip, knee, and ankle joints can be achieved independently for the right and left legs. It was found that different rostrocaudal positions of a small diameter electrode provided unique degrees of selectivity for different muscles. The rostral electrode, positioned lateral to the midline and centered rostrocaudally over the T10/T11 interspinous ligament, was the optimal position for RF and VL muscles, whereas the caudal electrode, centered over the T12/L1 ligament, was the optimal contact for TA, MG, and SL. However, optimal selectivity for the ST was more challenging to achieve. The caudal electrode was marginally better than other rostrocaudal positions, but this difference was non-significant. Despite the caudal segment being the most probable site for ST recruitment, it was observed that ST muscle recruitment was highly likely to occur at low stimulation amplitudes across all three segments. This consistent recruitment of ST and other hamstring muscles at different stimulation sites has been previously observed by other groups and has been attributed to their broader segmental innervation than other muscles. PRM reflexes of the hamstring muscles may not provide useful information in intraoperative monitoring to guide electrode placement. The results similarly suggest that responses from the other leg muscles should be prioritized when selecting the optimal electrode placement for a particular individual.

Interestingly, it was found that the middle electrode, which was centered at the T11/T12 vertebral level, did not provide optimal selectivity for any muscle. This finding is noteworthy because conventional tSCS protocols often center the electrode at this level. It is not suggested that this placement is incorrect or ineffective as if the objective were to target both proximal and distal muscles simultaneously by one stimulation site, and T11/T12 or T12/L1 would be ideal placements. Rather, it may not be the optimal position for achieving selective activation of specific muscle groups.

Despite this, it is not recommended to discard the T11/T12 position in the multielectrode configuration altogether. It may be notable in the context of multipolar configurations of tSCS, which have been shown to enhance recruitment selectivity further and prevent the unwanted activation of non-targeted muscles in eSCS. Therefore, while T11/T12 may not be the most selective electrode position for targeting specific muscles, it may still have important roles in optimizing tSCS protocols for further personalized improvements.

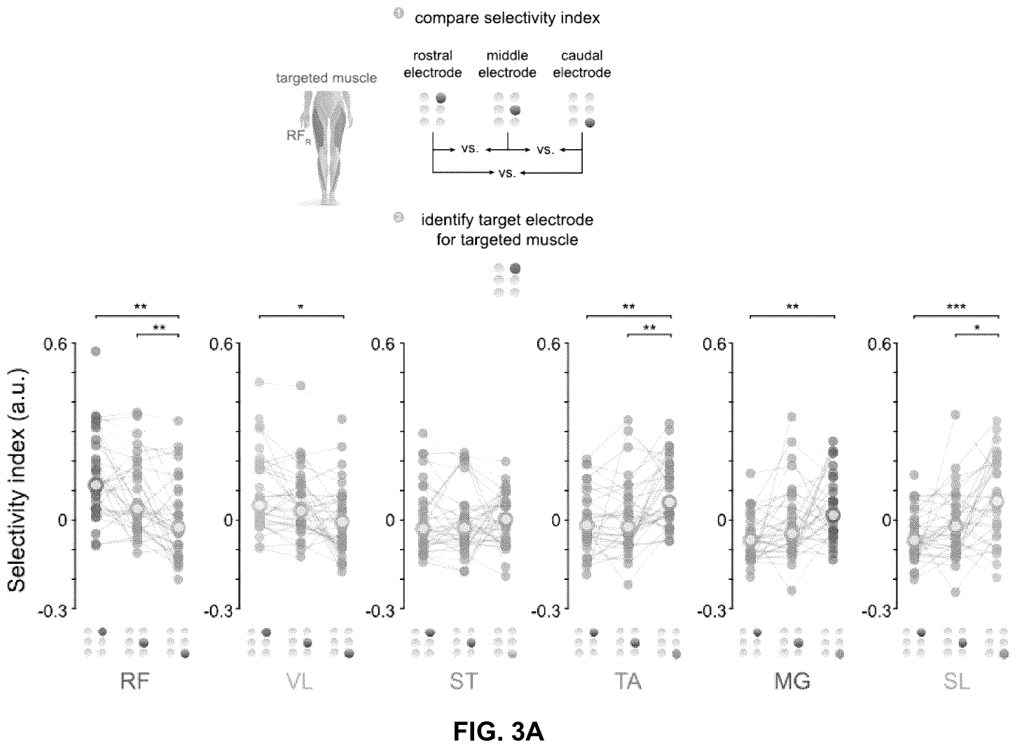

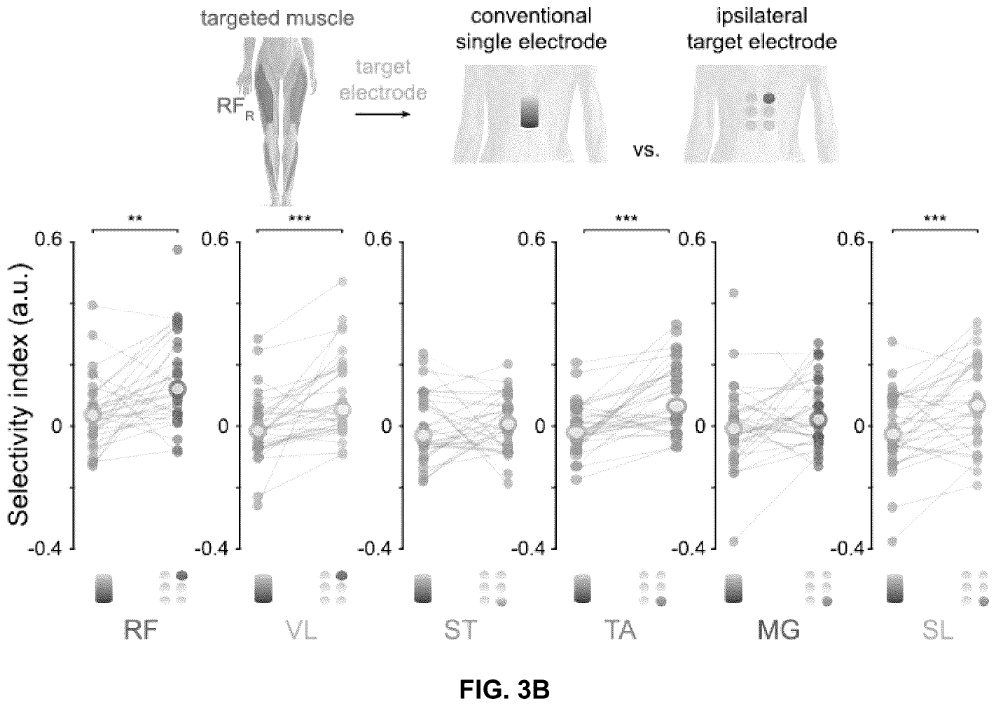

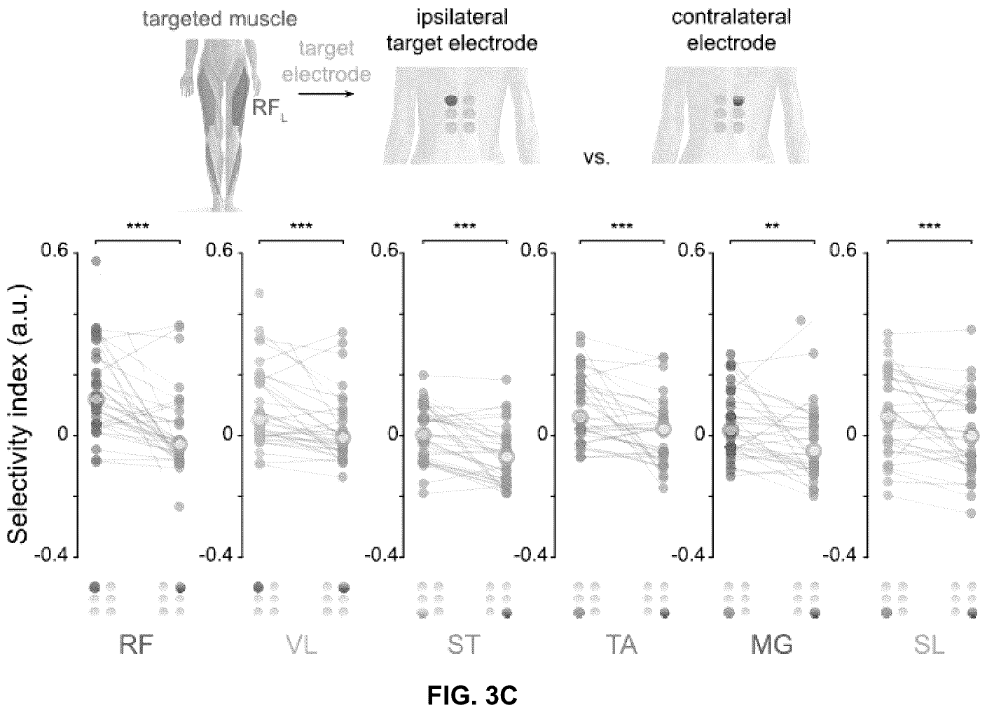

This study revealed that achieving optimal recruitment selectivity using tSCS is not universal across individuals and targeted muscles. As shown in FIG. 3B, the conventional tSCS electrode placement outperformed the target electrode in some participants' muscles. While the results provide a valuable starting point for improving muscle recruitment selectivity in individuals with neuromotor disorders, it is important to recognize that interindividual differences in neuroanatomy and neurophysiology, particularly in the damaged nervous system, will require personalized approaches. To optimize stimulation protocols for each individual's residual ability and specific needs, it is believed that it is beneficial to test muscle recruitment for each participant and verify that these activations are mediated by posterior root-muscle reflexes. Modifications in stimulation parameters such as amplitude, frequency, and pulse width should then be made and reported based on these initial activation thresholds and selectivity starting points.

Recent advancements in eSCS have demonstrated the potential of machine learning approaches to identify optimal stimulation parameters for targeting motor function rapidly, across subjects and species. Similarly, algorithms for automated calibration of stimulation parameters can be employed to determine clinically suitable tSCS settings across participants with different anatomies and neuromotor disorders. Therefore, these approaches hold promise for streamlining and optimizing the customization of stimulation parameters in tSCS applications.

The motor-enabling effects of SCS are primarily associated with the recruitment of proprioceptive afferent fibers in the posterior roots. However, because of the complex topographic anatomy in the lumbosacral spinal cord, non-invasive stimulation at different lumbosacral vertebral segments can excite both afferent and efferent pathways to different degrees. The engagement of each pathway is beneficial to both the immediate prosthetic effect that may be enabled by tSCS and the rehabilitation effect that may be observed through SCS-assisted therapy. Therefore, careful evaluation of evoked responses should be performed.

While direct recruitment of motor axons within the anterior roots, such as in functional electrical stimulation, can indeed produce desired movements in leg muscles, the artificial recruitment of large-diameter motor fibers makes it technically challenging to produce and sustain large forces. Moreover, the post-synaptic activation of motor fibers bypassing spinal and descending circuits limits the types of movements that can be performed to those that can be pre-programmed and prevents the voluntary modulation and neuroplasticity potential of these prosthetic effects. In contrast, the recruitment of afferent fibers by SCS enables interaction with descending and spinal circuits, which can enhance residual descending inputs and lead to a natural recruitment order that is fatigue-resistant and capable of sustaining the whole-body weight for extended periods. This presynaptic recruitment of primary afferents, in turn, promotes neuroplasticity, which is believed to mediate the neurological recovery observed during neurorehabilitation facilitated by SCS.

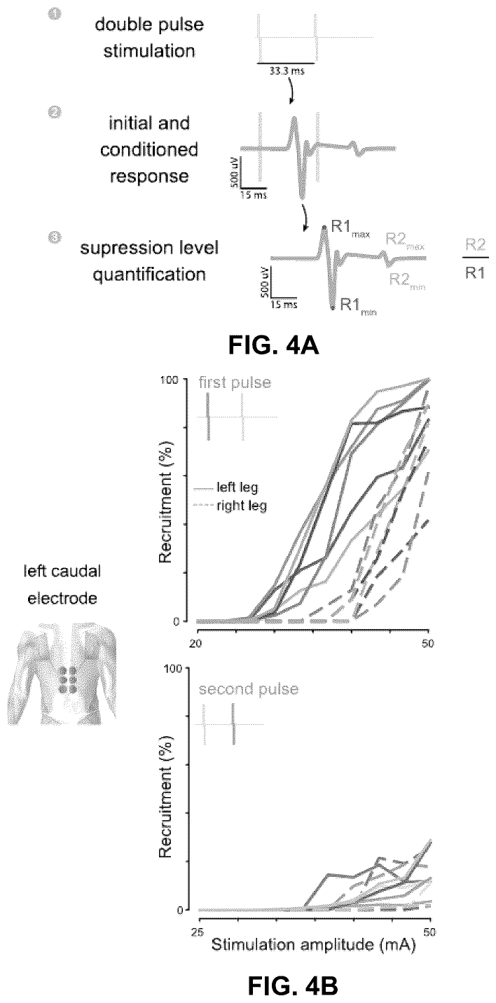

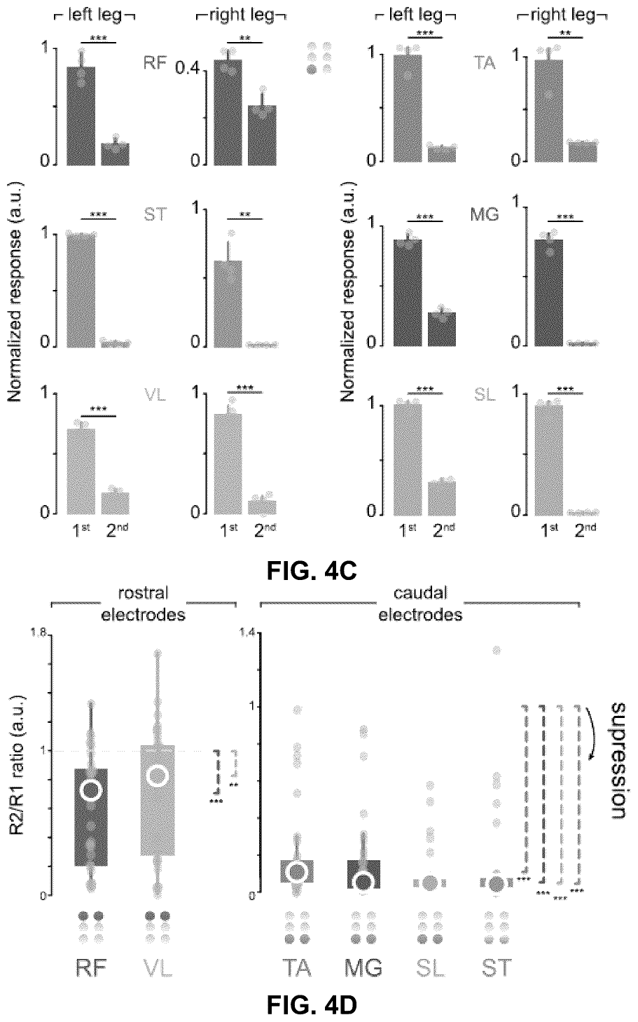

Overall, evoked responses by all target electrodes were mediated by PRM reflexes, as evidenced by the suppression of the conditioned response. However, the probability of efferent fiber recruitment differed substantially between L2-L4 innervated (RF, and VL) and L4-S2 innervated (ST, MG, TA, and SL) muscles (FIG. 4D). There were a few cases for proximal muscles in which the response average amplitude to the second pulse was higher than the average amplitude of the first response (values >1 in RF, ST, and VL). In contrast, there were no cases suggesting efferent recruitment in TA, MG, or SL, and only one case in ST. This suggests that higher stimulation amplitudes could be employed at caudal segments to target distal muscles with a reduced likelihood of recruiting motoneurons directly.

It should be noted that the degree of suppression was calculated at the maximum stimulation amplitude, which is associated with a greater likelihood of directly recruiting motor neurons. This selection was deliberate to facilitate a conservative analysis of recruitment mechanisms in a ‘worst-case’ scenario. Routine applications of tSCS use stimulation amplitudes between 0.8 and 1.2 times the motor threshold to enhance motor function after paralysis. As these amplitudes are considerably lower than saturation, it is expected that the probability of directly recruiting anterior roots in a therapy setting to be lower than those reported here. Nevertheless, because spine curvature can dramatically alter the probability of directly recruiting the anterior roots, verification of post-activation depression should be carefully confirmed in different therapeutic settings (e.g. sitting, standing, or in the position required to use a rehabilitation device).

Despite the marked effects of stimulation amplitude and sensory feedback on evoked responses, continuous SCS is commonly used in studies involving SCS-assisted neurorehabilitation. In this application, stimulation parameters are typically fixed at the onset of therapy and kept constant across different types of movements. This continuous, non-selective SCS largely diverges from the natural spatiotemporal activation patterns observed during movement and can disrupt the natural feedback from proprioceptive afferents, which is critical to enable the spinal regulation of movements during fine motor control. Although long-term training combined with continuous SCS can indeed induce improvements in motor function that persist even without stimulation, they may take several months to a year of intense rehabilitation to appear.

Spatial and temporal control of SCS aims to facilitate movements through the selective activation of specific motor neuron pools at the appropriate phases of movement. Recent clinical studies have shown that spatiotemporal eSCS can rapidly enable (within one week) powerful facilitation of walking, cycling, kayaking, and other dexterous activities in people with SCI as well as upper limb movements and grasping ability in people with stroke.

Combining long-term activity-based training with tSCS has been shown to improve standing and balance, and induce functional recovery that is comparable to that achieved through eSCS. Using multielectrode configurations of tSCS to selectively target facilitation in certain muscles during movements could help individuals learn to perform complex movements faster, potentially accelerating their recovery process. By speeding up the recovery process, these technologies could then become more accessible to individuals with SCI by reducing the amount of training required and, ultimately, the cost.

The invasive nature of eSCS offers advantages in terms of electrode placement and proximity to the spinal cord, enabling more precise targeting of specific neural structures and resulting in enhanced selectivity compared to tSCS. While direct comparative studies are necessary for a comprehensive analysis of selectivity differences between the two techniques, previous findings allow one to illustrate some similarities and distinctions between both approaches. In both eSCS and tSCS of the lumbosacral spinal cord, agonists and antagonist muscles are recruited simultaneously when targeting proximal or distal muscle groups.

In eSCS, various stimulation patterns have been explored to address the challenge of overlapping agonist and antagonist posterior roots. For instance, in multipolar electrode configurations, electrodes surrounding the cathode electrode are simultaneously stimulated as anodes. This approach is thought to help shield the distribution of the cathodic field and limit the unintended activation of the non-targeted muscles. However, it is important to note that this approach has not been specifically tested in tSCS, where the return electrode would still be positioned on the anterior side of the body. Therefore, testing is required to evaluate its effectiveness in the tSCS context.

In this work, the investigators aimed to improve selectivity in 12 muscles by using an electrode grid of only six electrodes. Theoretically, increasing the number of electrodes in multielectrode arrays with small-diameter electrodes could further improve recruitment selectivity. However, in the clinical application of this approach, where continuous 30 Hz stimulation may be delivered, it is crucial to consider the safety implications of an increase in power density. In this study, the recommended maximum power density for the Axelgaard electrodes may be 0.1 W cm−2.

In summary, it is demonstrated that the use of multielectrode tSCS results in enhanced rostrocaudal and unilateral selectivity compared to conventional tSCS. The optimal electrode position for targeting each leg muscle depends on the segmental innervation of its motor neuron pools within the spinal cord and allows for the targeted recruitment of specific muscle groups while minimizing the recruitment of other non-targeted muscle groups. As these are PRM-reflex-mediated responses, the interaction with descending and spinal neural circuits may enable the translation of this technology into effective therapies that aim to improve movement capacity in people with SCI.

One aspect of the present disclosure provides for a multielectrode-configured device that provides electrical transcutaneous spinal cord stimulation.

In the present disclosure, electrodes are placed along the spine or lateral to the spine. The present teachings include methods for stimulating muscle groups comprised of slowly ramping up the stimulation amplitude in all the electrodes to a baseline and then providing shorter ramp-up stimulations to spatially selective individual electrodes. In some aspects, the baseline stimulation amplitude is a certain percentage of the effective stimulation amplitude. The effective stimulation amplitude is the amount of stimulation amplitude necessary to provide a therapeutically effective amount of electrical stimulation.

The present teachings include descriptions of activating electrodes, in which the electrodes ramp-up from a baseline stimulation amplitude to an effective stimulation amplitude. In another aspect, the electrodes may be activated singularly or in combination with one of the other electrodes. In yet another aspect, the electrodes may be activated in sequence.

In accordance with other aspects, stimulation frequency at each electrode can be tuned to selectively target flexor vs extensor muscle groups at that body region. In other aspects, one range of frequencies can be used to target flexor muscles, and another range can be used to target extensor muscles. In yet other aspects, these frequency ranges can be either non-overlapping or have some level of overlap.

In some aspects, a non-invasive technological framework for activity-based rehabilitation that provides neuroprosthetic assistance according to each individual's residual function and specific needs is described. In other aspects, this strategy can be based on spatial, frequency, and amplitude control of tSCS. In accordance with another aspect, a multielectrode tSCS array to selectively target right vs. left legs and hip vs. ankle muscles is described. In yet another aspect, spatial control of tSCS allows for targeting of only those joints that require assistance without disrupting function in intact joints.

In the present disclosure, neuroanatomical differences between flexor and extensor muscles can be exploited to further improve selectivity within a joint using different tSCS frequencies. In some aspects, selective targeting of flexor and extensor muscles can be incorporated into the multielectrode tSCS and used in conventional electrodes to reinforce specific types of movements during single-joint exercises. In other aspects, tSCS can be tailored to provide assistance to agonist muscles with minimal activation of their antagonists.

In other aspects, the relationship between stimulation amplitudes, motor thresholds, and the observed neuroprosthetic effect of tSCS can be analyzed. In another aspect, the systems and methods described herein can characterize changes in stimulation amplitude, their rate of change, and their impact on perceptions of pain. In yet another aspect, the systems and methods described herein can provide clinicians and therapists with a framework to understand how to maximize motor function by tSCS while minimizing pain.

In some aspects, targeting specific muscle groups exclusively during the movement phases in which they should be engaged is described. In other aspects, spatiotemporal control of tSCS can include the control of a combination of spatial location, frequency, and amplitude. In yet other aspects, the control of spatial location, frequency, and amplitude can each independently improve current approaches in SCI by creating a stimulating framework that allows individuals with SCI to practice tSCS-assisted leg movements. In another aspect, the described systems and methods enable neuroscientists and engineers to probe the neural mechanisms of movement and motor control. In accordance with another aspect, the described systems and methods facilitate the ability to probe the neural and motor control systems as people with SCI exercise and adaptively learn to reorganize the control of their paretic limbs. In another aspect, non-invasive tSCS can be set up by individuals with SCI with the help of a family member or caretaker. In another aspect, the systems and methods described herein can create a stimulating practice framework to motivate individuals to keep an active body. In yet another aspect, the systems and methods described herein help individuals with SCI avoid the collateral effects of paralysis and recover some of their lost mobility.

### Computing Systems

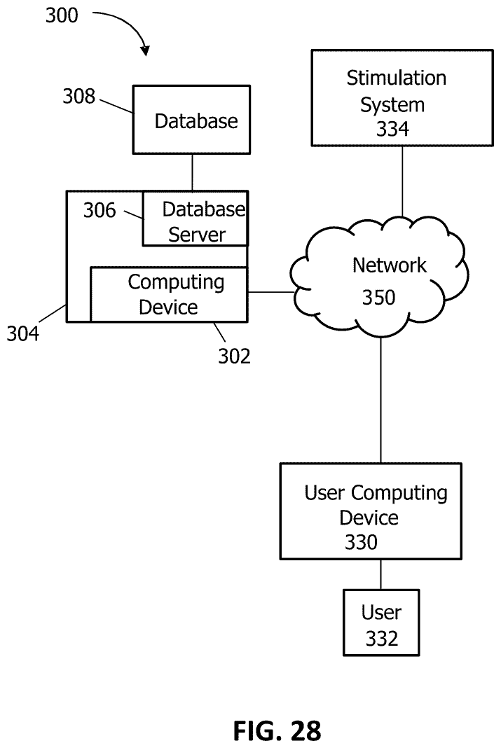

In various aspects, the disclosed stimulation methods may be implemented using a computing system or computing device. FIG. 28 depicts a simplified block diagram of the system for implementing the computer-aided method described herein. As illustrated in FIG. 28, the computing device 300 may be configured to implement at least a portion of the tasks associated with the disclosed stimulation methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330 and a stimulation system 334 as disclosed herein through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

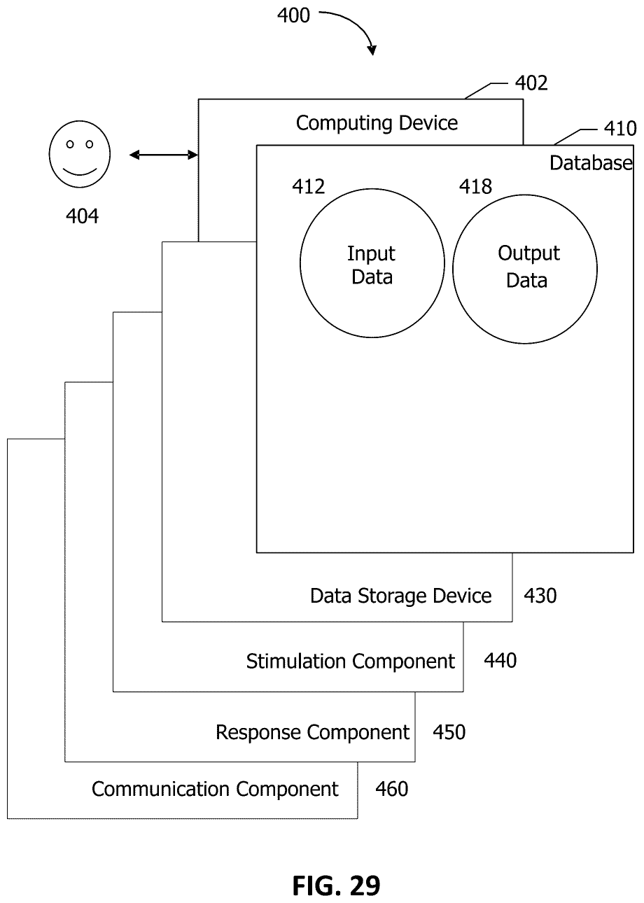

In other aspects, the computing device 302 is configured to perform a plurality of tasks associated with the disclosed computer-aided methods of performing tSCS stimulation as disclosed herein. In some aspects, the computing device 302, user computing device 330, and/or stimulation system 334, including, but not limited to, the disclosed tSCS system, may be operatively connected via a network 350. FIG. 29 depicts a component configuration 400 of computing device 402, which includes database 410 along with other related computing components. In some aspects, computing device 402 is similar to computing device 302 (shown in FIG. 28). A user 404 may access components of computing device 402. In some aspects, database 410 is similar to database 308 (shown in FIG. 28).

In one aspect, database 410 includes input data 412 and output data 418. Input data 412 may include data used to operate a tSCS system and to implement the implementation of electrode stimulation patterns as disclosed herein. Non-limiting examples of input data 412 include various signals used for stimulation, any parameters used to control the operation of a stimulation device for the tSCS methods as disclosed herein, as well as methods of operating a wearable EEG array and transforming patterns of detected EEG signals into signals indicative of a subjects intended movement. In another aspect, input data 412 can include data acquired by the stimulation system 334 and then re-inputted into the stimulation for feedback functionality. Output data 418 may include any responses to stimulation, including but not limited to physical human movements and associated detection signals, used to implement the stimulation and response methods as disclosed herein.

Computing device 402 also includes a number of components that perform specific tasks. In the exemplary aspect, computing device 402 includes a data storage device 430, a stimulation component 440, a response component 450, and a communication component 460. The stimulation component 440 is configured to implement the stimulation methods as described herein. The response component 450 is configured to acquire responses associated with stimulations implemented by the stimulation component 440 to implement the stimulation and response methods as disclosed herein. The data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 28) over a network, such as a network 350 (shown in FIG. 28), or a plurality of network connections using predefined network protocols such as TCP/IP (Transmission Control Protocol/Internet Protocol).

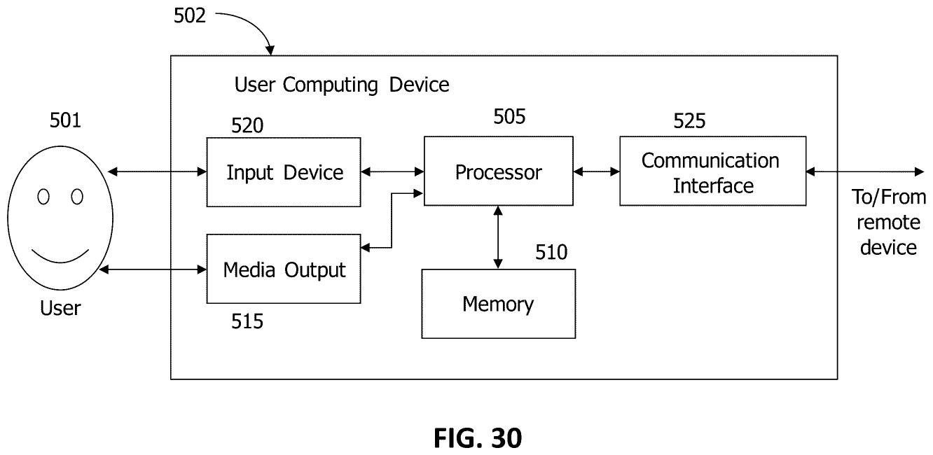

FIG. 30 depicts a configuration of a remote or user computing device 502, such as user computing device 330 (shown in FIG. 28). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

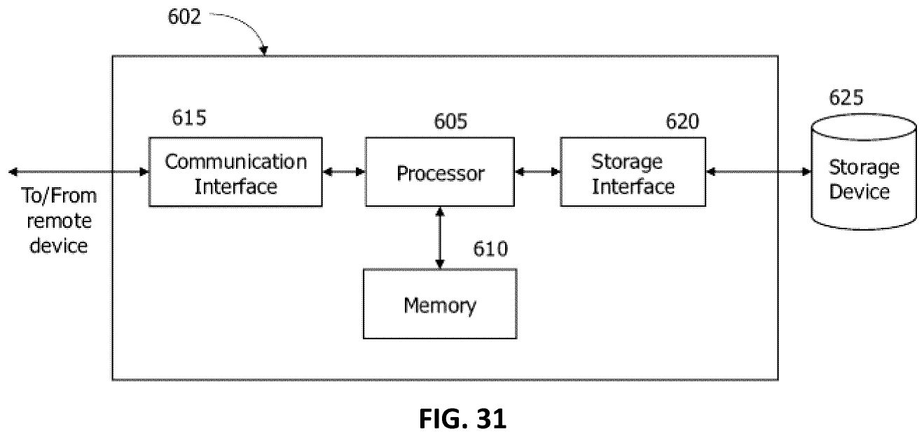

FIG. 31 illustrates an example configuration of a server system 602. Server system 602 may include, but is not limited to, database server 306 and computing device 302 (both shown in FIG. 28). In some aspects, server system 602 is similar to server system 304 (shown in FIG. 28). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 28) or another server system 602. For example, communication interface 615 may receive requests from a user computing device 330 via a network 350 (shown in FIG. 28).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIG. 30) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods.

The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above. In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics and animal behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: linear or logistic regressions, random forest classifiers, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship.

In yet another aspect, ML methods and algorithms are directed toward reinforcement learning, which involves optimizing outputs based on feedback from a reward signal. Specifically ML methods and algorithms directed toward reinforcement learning may receive a user-defined reward signal definition, receive a data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game AI, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

### Therapeutic Methods

Also provided is a process of a neuromodulation intervention, including but not limited to neuromotor disorders injuries, in a subject in need of administration of a therapeutically effective amount of electrical stimulation, so as to restore movement to paralyzed areas below an injury.

Methods described herein are generally performed on a subject in need thereof. A subject in need of the therapeutic methods described herein can be a subject having, diagnosed with, suspected of having, or at risk for neuromotor disorders, including but not limited to a spinal cord injury. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of electrical stimulation is, for example, an amount that would cause the desired therapeutic effect in a subject while minimizing undesired side effects. In various embodiments, an effective amount of electrical stimulation described herein can restore movement to paralyzed areas below an injury.

It will be appreciated by those skilled in the art that the unit content of treatment contained in an individual dose of each dosage form need not in itself constitute a therapeutically effective amount, as the necessary therapeutically effective amount could be reached by administration of a number of individual treatments.

Again, each of the states, diseases, disorders, and conditions, described herein, as well as others, can benefit from the methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of electrical stimulation can occur as a single event or over a time course of treatment. For example, electrical stimulation can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for neuromotor disorders.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Multielectrode Configuration of TSCS Resulted in Enhanced Rostrocaudal and Unilateral Selectivity Compared to Conventional TSCS

To characterize muscle recruitment enabled by conventional and multielectrode configuration electrodes, the following experiments were conducted. Leg muscle responses were recorded over a broad range of stimulation amplitudes, and motor thresholds for the conventional and multielectrode configurations were determined.

### Methods

The target vertebral segments T11/T12 were identified via manual palpation, with validation by a second experienced practitioner. The posterior iliac crest was first identified, and a transverse line was traced to the midline. The spinous process intersecting with this line was labeled with a surgical skin marker as the L4 spinous process. Interspinous ligaments were identified by palpation and labeled from L3/L4 to T9/T10. The skin around the midline from T10 to L2 was prepared with abrasive gel (NuPrep®, Weaver and Co. USA) using a Q-tip® in circular motions and wiped afterward with alcohol pads.

For conventional tSCS stimulation, a single 5×9 cm rectangular electrode (PALS Neurostimulation Electrode, Axelgaard Manufacturing Co., Ltd, USA) was placed in a centered position over the interspinous ligament of T11/T12 (FIG. 1A). Two interconnected 7.5×10 cm rectangular electrodes were placed on the abdomen bilaterally from the navel to serve as the return electrodes. All tSCS electrodes were treated with conductive spray (Signa® Spray, Parker Laboratories, Inc., USA).

Although palpation provided a useful starting point, anthropologic, clinical, and imaging studies have shown that variations in the number of vertebrae occur in 2%-23% of the population, and surface tSCS electrodes may often be repositioned to achieve activation of the targeted muscles. To ensure the accurate position of the conventional tSCS electrode over the lumbosacral enlargement, effective stimulation of segmental afferents was neurophysiologically confirmed by the elicitation of posterior-root muscle (PRM) reflexes bilaterally in L2 to S2 innervated myotomes, i.e. in quadriceps, hamstrings, calves, and tibialis anterior muscles. If either proximal or distal muscles could not be recruited at comfortable stimulation amplitudes, the electrode placement was adjusted by moving one segment up or down as necessary. Repositioning was performed on five participants, and the horizontal midline of the adjusted conventional electrode placement was then used as the rostrocaudal center for the multielectrode configuration (middle electrodes).

For the multielectrode treatment, six 3.2 cm diameter round electrodes were positioned 3 cm lateral to the midline and centered at the T11/T12 interspinous ligament (or at the center of conventional electrode if repositioned, FIG. 1A). Top, middle, and bottom row electrodes are referred to herein as the rostral, middle, and caudal electrodes, respectively. The top and bottom row electrodes were positioned to target the adjacent rostral and caudal interspinous ligaments, respectively. If electrode positions overlapped using this electron placement protocol (particularly in shorter participants), the electrodes were placed 1 cm longitudinally from the center row. Overall, there was an approximate distance of 1-2 cm between electrodes in the longitudinal direction. In the multielectrode condition, only the abdominal electrode positioned ipsilateral to the cathode was used as the return electrode.

Data was acquired through Wireless electromyography (EMG) sensors (Trigno® Avanti, Delsys Inc., USA) placed bilaterally according to SENIAM guidelines on the rectus femoris (RF), vastus lateralis (VL), semitendinosus (ST), tibialis anterior (TA), medial gastrocnemius (MG), and soleus (SL) muscles. The overlying skin was prepared using the same procedure as with the spinal electrodes, and electrodes were repositioned if the baseline noise was larger than 10 μV. An additional wireless sensor (Trigno® Analog Input Adapter, Delsys Inc., USA) was connected via a BNC cable to the biphasic stimulator's sync output for offline stimulation pulse alignment. Stimulation pulse triggering and amplitude were controlled via a data acquisition board (NI USB 6001, National Instruments, USA). EMG data was amplified using a data acquisition system (Trigno® Avanti Research+, Delsys Inc., USA; gain: 300; bandwidth 20-450 Hz), recorded at a sampling frequency of 2000 Hz, and displayed in real-time using custom-built software written in Python (v3.10).

Paired pulses tSCS was delivered using an isolated constant current stimulator (DS8R, Digitimer Ltd, UK) to generate a pair of charge-balanced, anodic leading, biphasic pulses of 1 ms per phase duration and an inter-stimulus interval of 33.3 ms using a train generator (DG2A, Digitimer Ltd.). Pulses of increasing stimulation amplitude were delivered to detect the motor threshold and saturation amplitude. The motor threshold was defined as the amplitude at which a peak-to-peak response amplitude of at least 20 μV within a latency of 10-30 ms was observed in any muscle. The saturation amplitude was defined as the lower of 1) the amplitude at which no further increase in response amplitude of the first recruited muscles was observed, or 2) the maximum amplitude tolerated by participants.

Recruitment curve recordings were obtained by increasing stimulation amplitude from 5 mA below the motor threshold to the saturation amplitude, with eight steps between amplitudes for a total of ten stimulation amplitudes. Four repetitions of double-pulse stimulation were performed at each amplitude. Threshold detection and recruitment curve recordings were repeated for each of the seven electrode configurations (one conventional tSCS configuration and six multielectrode tSCS configurations). The testing order was randomized for the multielectrode configurations.

Offline data analysis was performed using custom-built software written in Python and MATLAB® (The MathWorks Ltd, USA). Evoked responses were averaged across the four repetitions, with one average response waveform for each of the ten stimulation amplitudes. Evoked responses for each muscle were normalized to the maximum averaged evoked response in both electrode configurations (one normalization for conventional tSCS and one normalization across the six round electrodes). Data was normalized separately for each condition since the maximum attainable response amplitude for each muscle was determined by either saturation or discomfort at that electrode configuration. To validate the robustness of the results to the normalization process, data were re-analyzed with one normalization across all conditions.

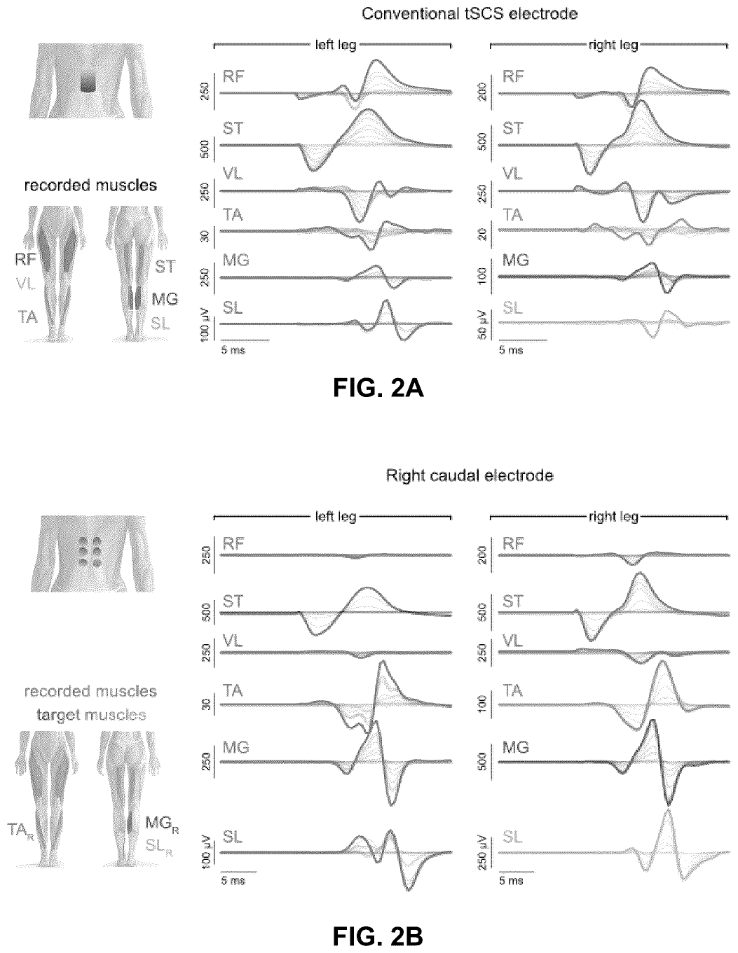

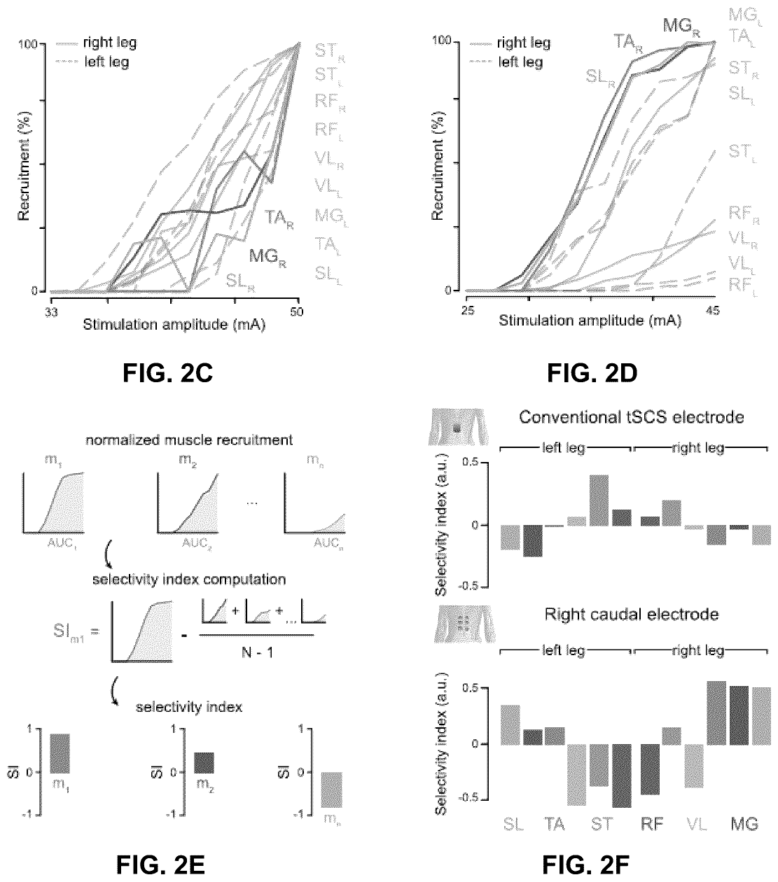

Selectivity index: To quantify the degree of muscle recruitment selectivity enabled by each electrode position, a recruitment curve for each muscle was first generated. This curve represented the muscle's peak-to-peak response as a function of stimulation amplitude. The recruitment for each muscle was then quantified as the area under the curve (AUC) of its recruitment curve normalized to the maximum possible recruitment i.e. maximal recruitment at all stimulation amplitudes. Last, the selectivity SI of muscle m was computed as the normalized recruitment RECm of that muscle minus the average normalized recruitment of M−1 muscles, excluding the considered muscle, m (FIG. 2E). Conceptually, the selectivity index reflects the normalized recruitment of a given muscle compared to the average activation of all other muscles:

$${SI}_{m} = {REC}_{m} - \frac{{\sum }_{n \neq m}^{M} {REC}_{n}}{M - 1}$$

The selectivity index varied from −1 to 1 and reflected the activation of a targeted muscle compared to the average activation of all other muscles, with a value of 0 indicating that a given muscle was recruited equally to the average of all other muscles. This computation was performed for each muscle, electrode configuration, and participant.

To identify the target electrode for each muscle, the selectivity index enabled by three electrode positions ipsilateral to the target muscle were compared (rostral: T10/T11, middle: T11/T12, caudal: T12/L1) and the electrode with the highest median selectivity index was selected as the target electrode for that muscle. The target electrode was compared to the conventional tSCS electrode to evaluate whether the target electrode enabled a significantly higher selectivity index for the targeted muscle and whether the selectivity index by the target electrode was higher than its contralateral counterpart (i.e. the small-diameter electrode at the same rostrocaudal level on the contralateral side).

### Results

Overlaid EMG responses to the first stimulation pulse at increasing amplitudes for the conventional and right caudal electrodes are shown for a representative participant (FIG. 2A, FIG. 2B). Notably, response latencies were progressively higher for caudally innervated muscles in both electrode configurations. This increase in latency was in agreement with previous observations, suggesting that the increase in latency was due to the distance between motor neuron pools and the innervated muscle rather than due to the electrode location. Identification of the nature of the responses based on their latencies was not always definitive. Therefore, existing neurophysiological methods were applied to further analyze these responses.

To understand the relationship between muscle recruitment and stimulation amplitude, the peak-to-peak amplitude of the averaged responses for each recorded leg muscle was calculated (FIG. 2C, FIG. 2D). The degree of muscle recruitment was proportional to the stimulation amplitude. However, the degree of recruitment for a given muscle at a given amplitude differed across electrode configurations. Stimulation by the conventional tSCS electrode configuration resulted in broad non-selective muscle recruitment, as reflected by similar motor thresholds across rostral, caudal, and bilateral muscles (FIG. 2C). In contrast, motor thresholds were lower in a subset of muscles recruited by the right caudal electrode, and their recruitment level was close to saturation by the motor threshold amplitude of the others (FIG. 2D), as evidenced by differences in recruitment level at ˜40 mA. The left leg distal muscles (TA, MG, SL) were also recruited at low amplitudes by the right caudal electrode in this participant. The targeted recruitment of a particular muscle group by the multielectrode configuration was commonly observed to be accompanied by some undesired recruitment of the other unilateral muscle group (recruitment of right leg proximal muscles in the targeting of right leg distal muscles or vice versa), or the contralateral muscle group. Therefore, recruitment selectivity across muscles and electrode configurations was further quantified.

To better interpret the degree of muscle recruitment selectivity enabled by each electrode position, the selectivity index for all muscles was computed (FIG. 2E). Selectivity indices for the conventional and right caudal electrodes in this participant are shown in FIG. 2F. The conventional tSCS electrode primarily enabled the recruitment of bilateral proximal muscles (RF, ST, VL), as indicated by a positive selectivity index. The AUC of the recruitment curve for distal muscles (TA, MG, SL) was lower than all others, resulting in a low and negative selectivity index for these muscles. In contrast, for the multielectrode configuration, the selectivity indices of distal muscles of the targeted (right) and non-targeted (left) legs were higher than most other muscles. Moreover, the relative activation of proximal muscles in both legs was lower than that for all other muscles, resulting in a low selectivity index for the non-targeted proximal muscles in this participant. To understand trends in recruitment selectivity that were common across participants, group-level analyses were performed.

### Example 2—Rostrocaudal and Ipsilateral Positioning Over Muscle-Specific Motor Neuron Pools Enhance Recruitment Selectivity of Targeted Muscle Groups

To identify the optimal electrode position to target each recorded leg muscle, the following experiments were conducted. The selectivity index as defined in Example 1 was computed for all muscles and electrode configurations across participants. The selectivity indices enabled by each electrode's rostrocaudal position (rostral, middle, or caudal) ipsilateral to the targeted muscle were then compared (FIG. 3A). Selectivity indices enabled by the multielectrode configuration depended on an electrode's rostrocaudal position for most recorded muscles, as quantified by the Friedman effect of electrode's rostrocaudal position in RF (χ2=9.75, p=0.008), VL (χ2=15.06, p <0.001), ST (χ2=0.063, p=0.969), TA (χ2=10.94, p=0.004), MG (χ2=6.06, p=0.048), and SL (χ2=10.56, p=0.005). Statistical significance for comparisons across electrode positions is shown above each muscle. The electrode with the highest median selectivity index was taken as the target electrode for that muscle for all subsequent analyses. Results for normality using the Kolmogorov-Smirnov test can be found in Appendix II.

It was hypothesized that the target ipsilateral electrode in the multielectrode configuration could enhance the recruitment selectivity of a targeted muscle group compared to conventional tSCS (FIG. 1D). To test this hypothesis, the selectivity index for each muscle enabled by the conventional tSCS and the ipsilateral target electrode were compared (FIG. 3B). Overall, the target electrode in the multielectrode configuration significantly enhanced recruitment selectivity compared to the conventional electrode for most muscles: Wilcoxon signed rank effect of RF (W=99, p=0.002), VL (W=43, p<0.001), ST (W=228, p=0.500), TA (W=54, p<0.001), MG (W=175, p=0.096), and SL (W=94, p=0.001).

To test whether the improvements in muscle recruitment selectivity could be achieved by positioning the small diameter electrode at the right rostrocaudal level regardless of the electrode's lateral position, the selectivity indices between the target ipsilateral electrode and its contralateral counterpart were compared (FIG. 3C). As an example, the selectivity index of the right RF when targeted by the right rostral electrode was compared to the selectivity index of the same muscle targeted by the left rostral electrode. Overall, the ipsilateral target electrode significantly enhanced recruitment selectivity compared to its contralateral counterpart: Wilcoxon signed rank effect of RF (W=54, p<0.001), VL (W=80, p<0.001), ST (W=55, p<0.001), TA (W=75, p<0.001), MG (W=100, p=0.002), and SL (W=65, p<0.001).

Although selectivity index values for individual participants slightly varied when normalization of peak responses was performed across all electrode configurations, the general trend across electrodes remained the same. The target electrode in the multielectrode configuration was the same for each muscle and achieved a higher level of selectivity than the conventional electrode and its contralateral counterpart. These results indicated that positioning small-diameter surface electrodes over the predicted locations of a muscle's motoneuron pools in a rostrocaudal direction while maintaining laterality improved the recruitment selectivity of that muscle.

### Example 3—Elicited Responses with Multielectrode Configurations are Mediated by Posterior Root-Muscle Reflexes

To evaluate the potential use of tSCS for recovery of motor function below a spinal injury, the following experiments were conducted. The posterior root-muscle (PRM) reflex was used to confirm the effective position of the stimulating electrode over the spinal cord. Without being limited to any particular theory, it was hypothesized that if elicited responses were indeed mediated by PRM reflexes, compound muscle action potentials to the test (second) pulse would reflect significant depression, a hallmark behavior of reflex responses.

### Methods

Paired pulse suppression. The amount of suppression by post-activation depression was calculated as the ratio between the second (R2) to the first (R1) response amplitudes (FIG. 4A). An R2/R1 ratio lower than 1 indicates that the amplitude of the evoked response to the second pulse was lower than the amplitude of the first response. The amount of suppression has been shown to be inversely related to the stimulation amplitude, so suppression at the highest stimulation amplitude was quantified, where the amount of suppression should be the lowest. This condition was selected to facilitate a conservative analysis of recruitment mechanisms in a ‘worst-case’ scenario.

### Results

Recruitment curves for the first and second pulses delivered through the left rostrocaudal electrode in a representative participant are shown in FIG. 4B. The amplitude of the response to the second pulse was largely suppressed in most muscles. However, the amount of suppression was inversely proportional to stimulation amplitude. Therefore, the level of suppression for each electrode at the highest stimulation amplitude was quantified, where the amount of suppression was generally the lowest. A comparison in response amplitudes between the first and the second pulse for the left caudal electrode in the same representative participant is shown in FIG. 4C. Overall, there was a significant reduction in response amplitude to the second stimulation by the left caudal electrode in that participant.

To analyze this effect across participants, the level of suppression was computed as the ratio between the first and second responses. A ratio smaller than one would indicate response suppression, i.e. that the response to the second stimulation pulse was lower than the response to the first pulse. The amount of suppression for each muscle, when targeted by its optimal electrode, is shown in FIG. 4D. There was a significant amount of post-activation depression of the evoked response by the paired-pulse paradigm at each optimal electrode configuration: one-sided 1-sample Wilcoxon signed rank test in RF (W=489, p<0.001), VL (W=407, p=0.004), ST (W=527, p<0.001), TA (W=528, p<0.001), MG (W=528, p<0.001), and SL (W=528, p<0.001). A significant amount of suppression was also observed in the conventional tSCS electrode treatment, consistent with previous reports. These results served as a neurophysiological confirmation that the optimal multielectrode configuration enables the effective stimulation of the respective segmental afferents in the targeted leg muscles.

### Example 4—Recruitment Probability for Each Muscle Reflected the Segmental Distribution of its Motor Neuron Pools in the Spinal Cord

To understand how different electrode configurations targeted the posterior roots projecting to spinal cord segments containing the motor neurons responsible for activating the hip, knee, and ankle joints, the following experiments were conducted. A neuroanatomical map of spinal cord activation for each electrode position was developed. The probability of activating each muscle at each electrode position was first calculated by averaging the normalized recruitment of that muscle across participants.

### Methods

The probability of recruiting a given muscle was computed for each electrode configuration as the normalized AUC of its recruitment curve at that electrode position. AUC values for each muscle were normalized to the AUC in the electrode that achieved the highest level of recruitment. The recruitment probability for each muscle was then computed as the average normalized recruitment across participants. In this computation, a recruitment probability of 1 would indicate that that muscle was maximally recruited at that electrode position for all participants.

Recruitment probability estimates were used to model the activation of motoneuron pools in each spinal segment Sᵢ as a linear combination of the normalized leg muscle recruitment Mⱼ, and Wᵢⱼ, the expected segmental distributions of motoneuron pools innervating muscle j at spinal segment Sᵢ.

$${S}_{i} = \frac{{\sum }_{muscles} {W}_{i , j} {M}_{j}}{{\sum }_{muscles} {W}_{i , j}}$$

The coefficients Wᵢⱼ were obtained from a constructed innervation probability map. The constructed innervation probability map included qualitative and quantitative data from thousands of participants in anatomical textbooks and electrophysiological studies. Probability estimates for the VL muscle were derived from a separate anatomical study showing similar innervation in quadriceps muscles within the L2-L4 spinal segments. The resulting spinal activation values were interpolated and superimposed onto a 2D image of the human lumbosacral spinal cord.

Statistical analyses were performed using the SciPy statistics toolbox (v1.8.1) for Python. Data for left and right leg muscles were pooled together for analysis. Normal distribution was tested using the Kolmogorov-Smirnov test. As a great majority of datasets were not normally distributed, non-parametric statistical tests were used. A Friedman test for repeated measures was performed to determine the effect of the multielectrode rostrocaudal vertebral level (rostral, middle, and caudal) on the selectivity index. A Wilcoxon signed-rank test with a Bonferroni correction for multiple comparisons was then used to determine significant differences in selectivity index across electrode positions. Separate Wilcoxon signed-rank tests were used to compare the selectivity index enabled by the target electrode against the conventional tSCS, as well as against its contralateral counterpart. A Wilcoxon rank sum test was used to test for significant suppression in each muscle by its target electrode.

### Results

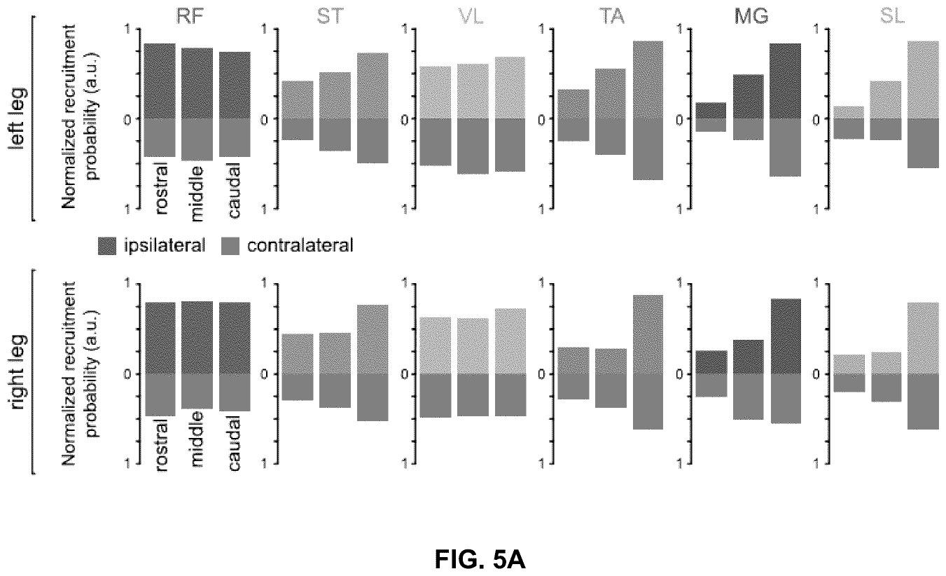

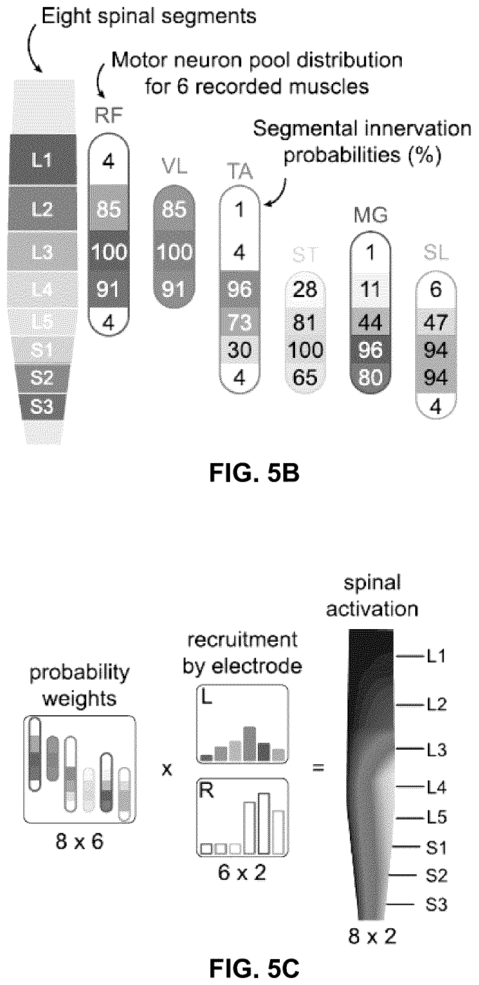

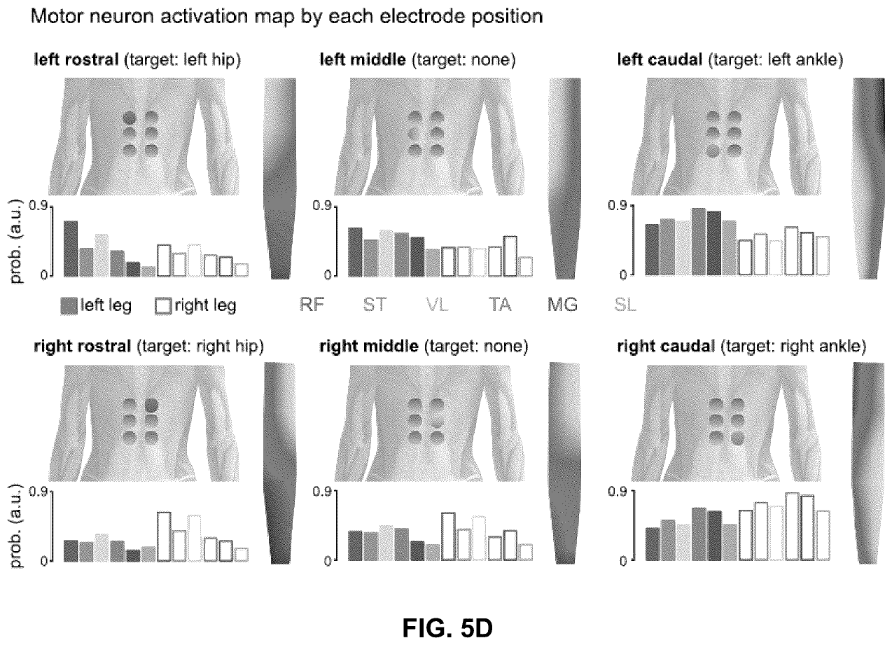

Recruitment probabilities for electrodes located ipsilaterally and contralaterally to the targeted muscles at different rostrocaudal positions are shown in FIG. 5A. Next, an atlas of the expected anatomical locations for the motor neuron pools associated with the recorded leg muscles was then compiled (FIG. 5B). Finally, the recruitment probability of each muscle was projected onto its respective innervation probabilities at each spinal segment (FIG. 5C). The resulting spinal activation maps elicited by each multielectrode position are shown in FIG. 5D.

The analysis of recruitment probability across participants revealed a high similarity between the optimal electrode configurations and the recruitment of the posterior roots projecting to the targeted spinal cord regions involved in the activation of the hip and ankle joints. For example, the left rostrocaudal electrode predominantly activated upper left lumbar segments (FIG. 5D, top left panel), while the right caudal electrode activated motor neuron pools located around right sacral segments (FIG. 5D, bottom right panel). It is important to note that the combination of the segmental innervation probabilities and the muscle recruitment probability was agnostic to the electrode configuration. Nevertheless, the spinal activation maps closely reflected the cathode location for that configuration.

### Example 5—Muscle Electromyography (Emg) Activity and Transcutaneous Spinal Cord Stimulation Amplitude During Training Right Leg Extension Trial in a Representative Participant

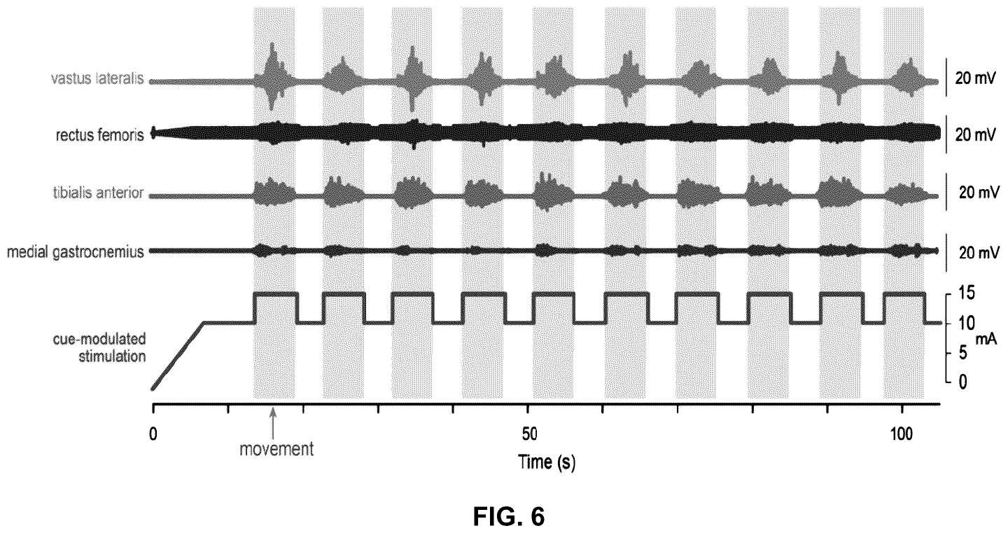

To develop a stimulation protocol to ensure tolerable jumps in stimulation amplitude at the appropriate phases of movement, the following experiments were conducted. As shown in FIG. 6, stimulation amplitude was increased from 0 to a resting stimulation amplitude of 10 mA over the first 7 seconds of each trial. Stimulation amplitude was then remotely controlled and timed to movement instructions. Stimulation was alternated between the rest intensity (10 mA) between movement cues and movement intensity (15 mA) during the movement cues.

The stimulation protocol described above was better tolerated by subjects as compared to an existing stimulation protocols in which stimulation was alternated between a rest intensity (0 mA) between movement cues and movement intensity (15 mA) during the movement cues.

The results of these experiments demonstrate that reducing the change in stimulation amplitude used to recruit muscle group activity in tSCS was associated with enhanced tolerability relative to existing tSCS stimulation protocols.

### Example 6—Muscle Electromyography (Emg) Activity and Transcutaneous Spinal Cord Stimulation Amplitude During Training Right Leg Extension Trial in a Representative Participant

To develop a non-invasive brain-spine interface to facilitate leg movements based on brain-decoded commands for people with SCI, the following experiments were conducted. It was hypothesized that movement-related desynchronizations in α, low β, and β could be used to predict knee extension onset from EEG signals.

A linear discriminant analysis (LDA)-based neural decoder was built that received 480 EEG spectral, temporal, and spatial features during movements of participants. Eight unimpaired participants were fitted with the LDA-based decoder and instructed to extend their knees based on a timed cue. To ensure that predictions of movement onset were not related to movement or cue-related artifacts, the ability of the neural decoder to generalize to imagined and uncued movements was tested. Finally, movement onset detections were linked to the delivery of tSCS on 10 unimpaired individuals similarly instructed to extend their knees based on a timed cue.

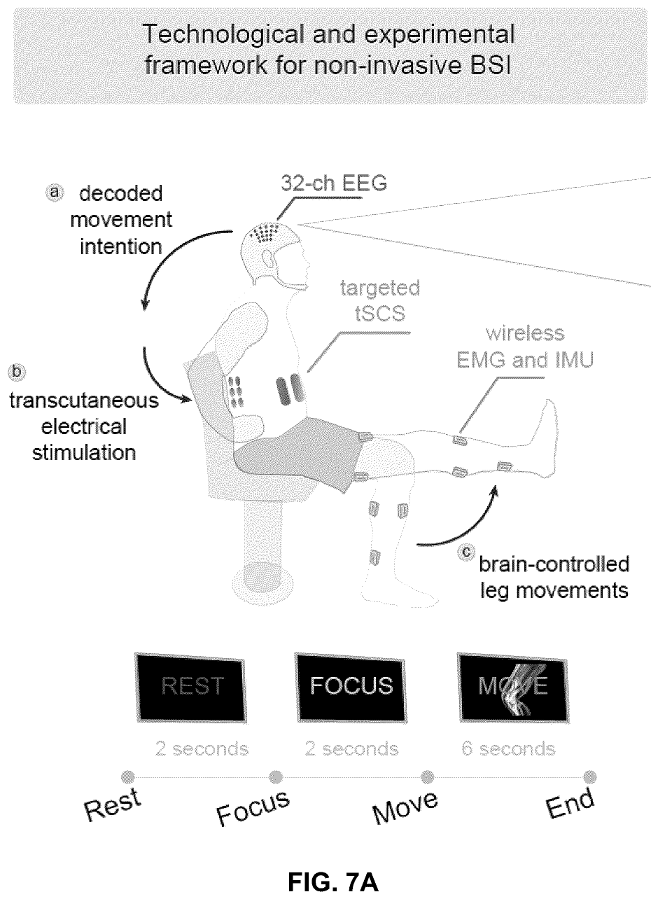

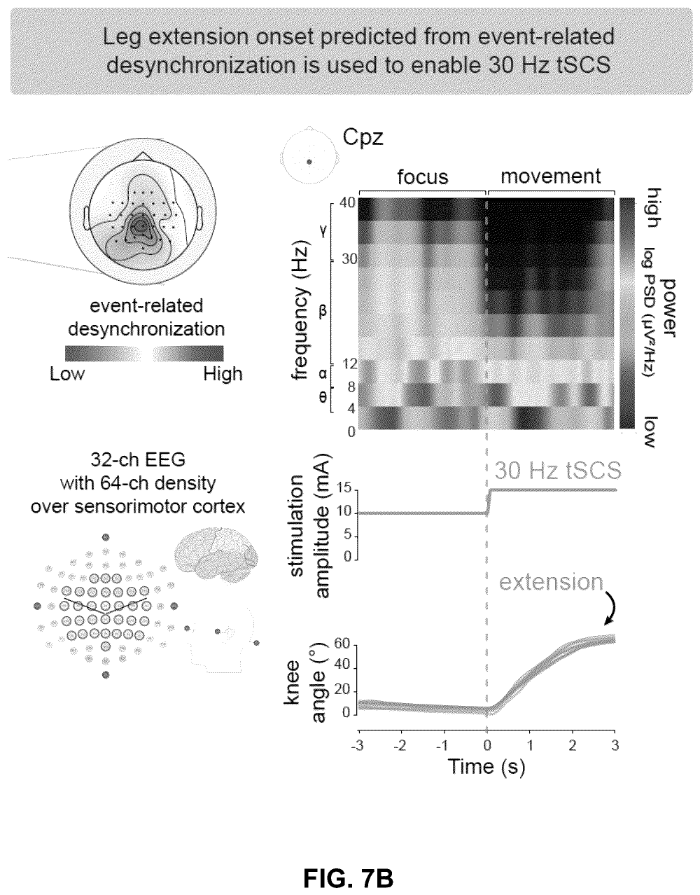

The linear discriminant analysis (LDA)-based neural decoder was integrated into a non-invasive brain-spine interface (BSI) to facilitate leg movements based on brain-controlled spinal cord stimulation. A schematic of the technological and experimental framework for non-invasive BSI using the BSI is shown in FIG. 7A, wherein leg extension onset predicted from event-related desynchronization was used to enable 30 Hz tSCS (FIG. 7B).

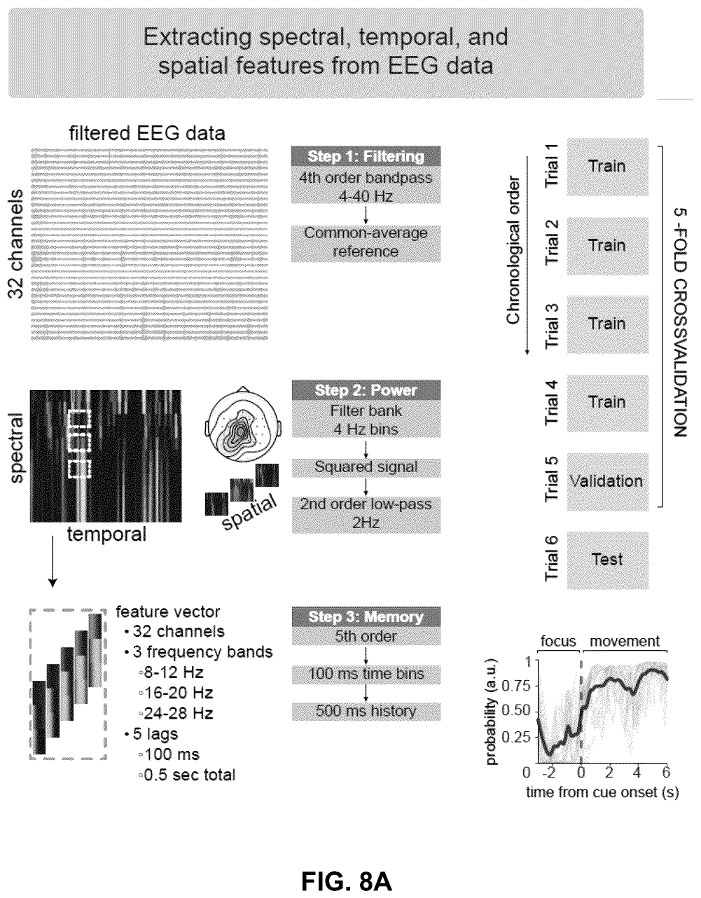

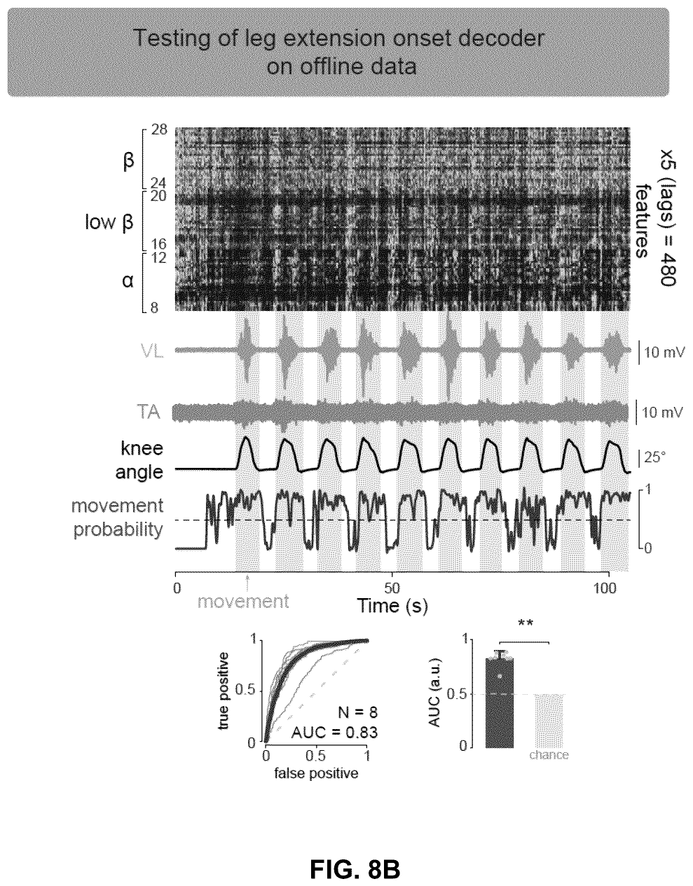

The neural decoder based on linear discriminant analysis (LDA) classifier enabled the prediction of movement onset based on the 480 neural features, including spectral, temporal, and spatial features extracted from EEG data (FIG. 8A). Testing of the leg extension onset decoder on offline data was performed, as illustrated in FIG. 8B.

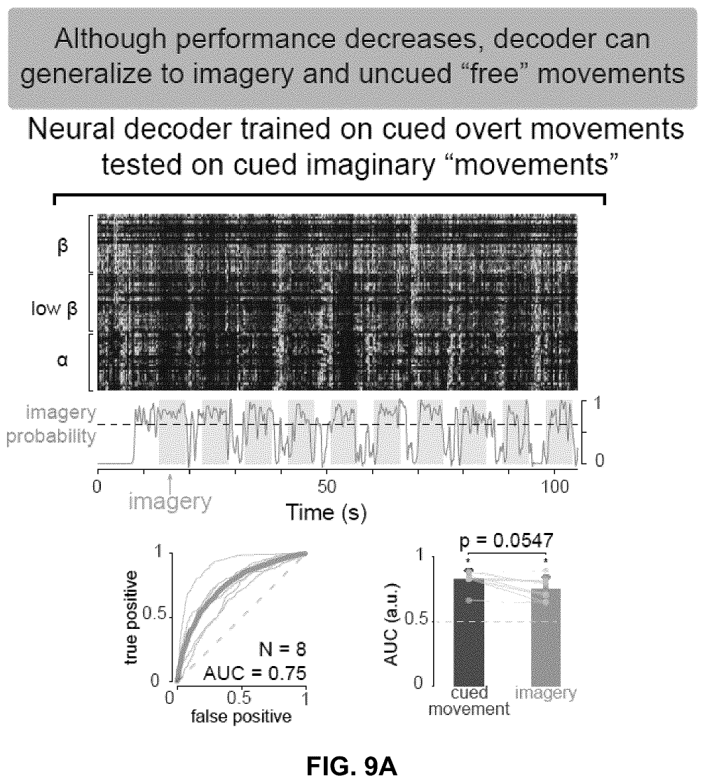

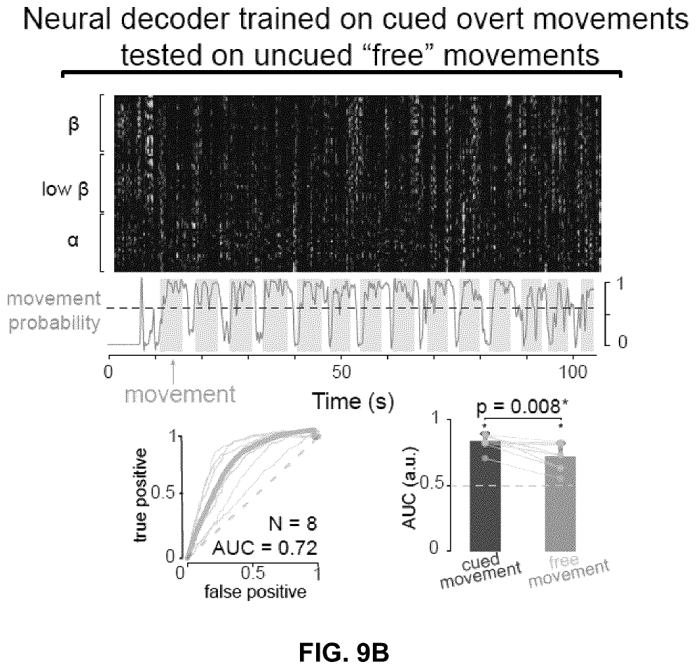

Validation against movement-related and cue-related artifacts and real-time testing of brain-controlled stimulation reinforcing right leg extension was also performed. Although performance decreased, the decoder demonstrated the capability to generalize to imagery and uncued “free” movements. To achieve this, the neural decoder was first trained on cued overt movements and tested on cued imaginary “movements” (FIG. 9A). The neural decoder was then trained on cued overt movements and tested on uncued “free” movements.

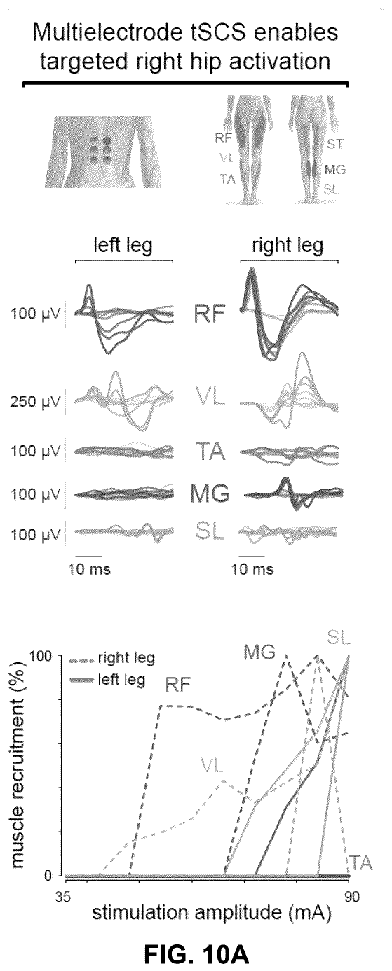

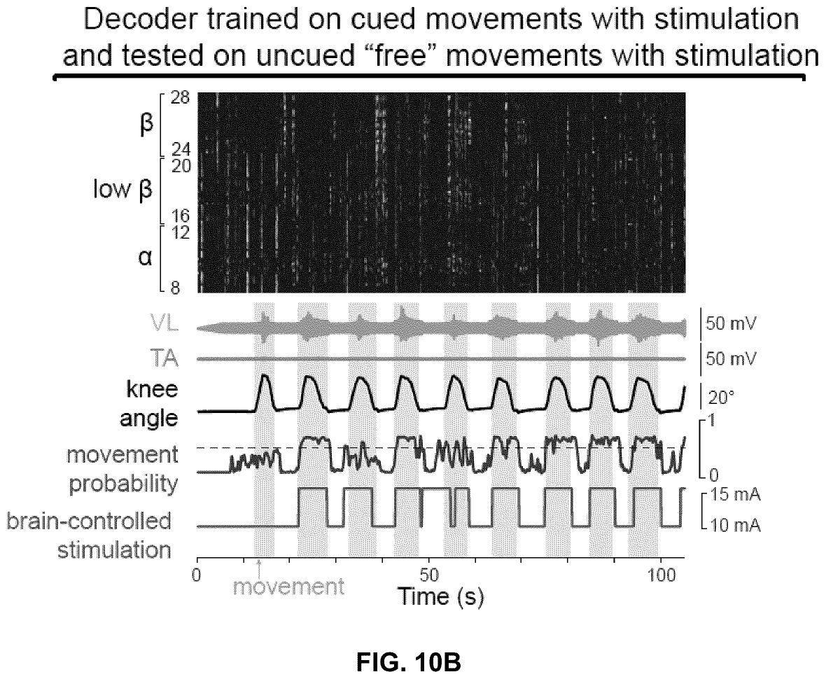

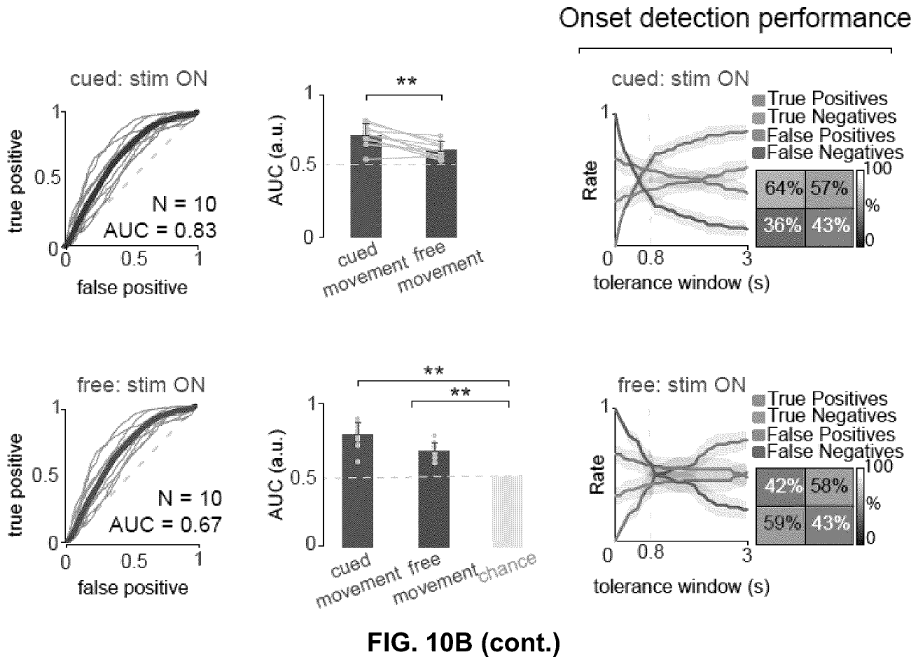

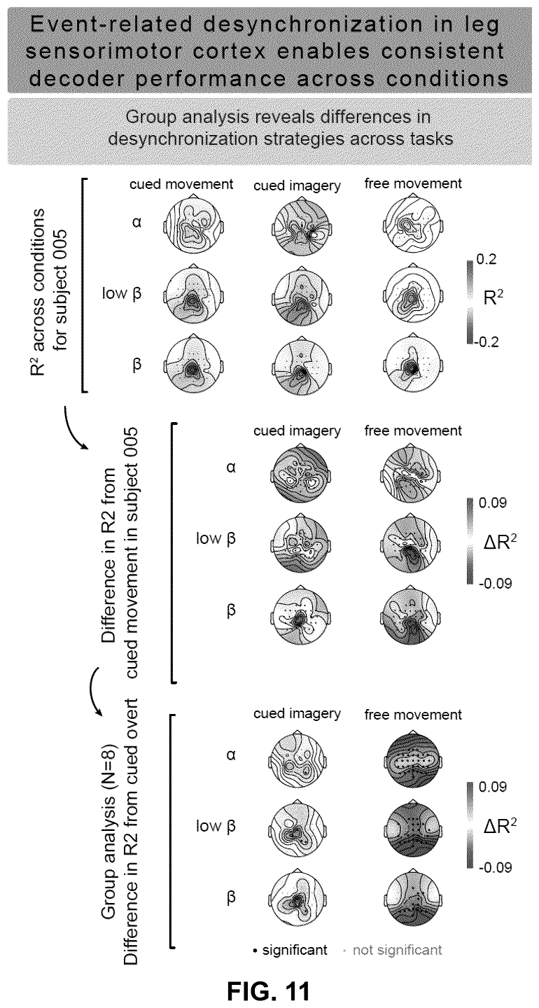

The brain-controlled real-time tSCS was shown to be synchronized with uncued leg extension movements. However, perceived accuracy depended on tolerance for variability in the timing of stimulation onset. Importantly, multielectrode tSCS enabled targeted right hip activation (FIG. 10A), and the decoder was trained on cued movements with stimulation and tested on uncued “free” movements with stimulation (FIG. 10B). Further, event-related desynchronization in the leg sensorimotor cortex enabled consistent decoder performance, as group analysis revealed differences in desynchronization strategies across tasks (FIG. 11).

The results of these experiments demonstrated the successful decoding of leg extension cues based on the analysis of EEG neural signals. Event-related desynchronization in the sensorimotor cortex enabled accurate prediction of leg extension onset with an average AUC of 0.83 across 8 unimpaired participants. As the neural decoder could generalize to imagery and uncued trials, the likelihood that successful decoder performance is due to movement or cue-related artifacts is low. However, the addition of brain-controlled stimulation resulted in lower decoder performance.

Implantable BSIs based on ECOG and epidural SCS enable long-term improvements in motor function when paired with activity-based training. Non-invasive technologies could leverage these results to facilitate single-joint movements in a physical therapy setting.

### Example 7—Transcutaneous Spinal Cord Stimulation (TSCS)

Spinal cord injury (SCI) is a life-altering event that leads to long-lasting motor impairment, and currently, there is no cure for paralysis. Recent work in epidural spinal cord stimulation (eSCS) by myself and others showed that this neuromodulation technique in combination with activity-based training has led to unprecedented improvements in motor function in the chronic stage of paralysis (>1 year post-injury). Stimulation of the lumbosacral enlargement below the injury provides a prosthetic effect that enables the activation of previously paralyzed leg muscles. This prosthetic effect of eSCS enables the prolonged voluntary activation of paralyzed muscles in a physical therapy setting, and this long-term practice with eSCS and activity-based training has resulted in increases in clinical measures of function in chronic SCI. However, the invasive nature of eSCS, the large group of experts needed to enable eSCS, and the high associated costs may limit eSCS from becoming an accessible therapy for the millions of people living with paralysis.

Transcutaneous SCS (tSCS) offers a promising non-invasive alternative to engage paralyzed muscles below the injury by activating the same neural circuits as eSCS. In conventional applications of tSCS, a single electrode is positioned over the T11/T12 interspinous ligament and centered at the midline (FIG. 1D). Continuous stimulation at 30 Hz is then used to facilitate motor function (FIG. 1E). Recent work suggests that positive rehabilitative outcomes may also be achievable through this non-invasive approach. However, the low selectivity of tSCS compared to eSCS may limit the types and number of movements that it can enable, and thus reduce its potential applications in exercise-based rehabilitation strategies.

Despite the marked effects of stimulation frequency, amplitude, and sensory feedback on evoked responses, continuous SCS is commonly used in studies involving SCS-assisted neurorehabilitation. Stimulation parameters are typically fixed at the onset of therapy and kept constant across different types of movements. This continuous, non-selective SCS largely diverges from the natural spatiotemporal activation patterns observed during movement, and it can disrupt the natural feedback from proprioceptive afferents, which is critical to enable the spinal regulation of movements during fine motor control. Although long-term training combined with continuous SCS can indeed induce improvements in motor function that persist even without stimulation, they may take several months to a year of intense rehabilitation to appear. Spatial (FIG. 1F) and temporal (FIG. 1G) control of eSCS aims to facilitate movements through the selective activation of specific motor neuron pools at the appropriate phases of movement. Recent clinical studies have shown that spatiotemporal eSCS can rapidly enable (within one week) powerful facilitation of dexterous activities such as walking, cycling, swimming, and kayak paddling in people with SCI. However, spatiotemporal control of tSCS has not yet been achieved. Therefore, the scope of rehabilitative activities that can be practiced using non-invasive SCS technologies, as well as the speed at which individuals can engage in them, remain limited.

Combining long-term activity-based training with tSCS has been shown to improve standing and balance, as well as inducing functional recovery that is close to that achieved through eSCS. If muscle recruitment selectivity of non-invasive tSCS can be improved, it may also be possible to selectively target individual muscle groups to facilitate muscle contractions at the appropriate phases of movement. This prosthetic effect would enable the early practice of dexterous activities in rehabilitation, potentially accelerating the recovery process. Therefore, tSCS technologies could then become more accessible to individuals with SCI by reducing the amount of training required and, ultimately, the cost. The data suggest that achieving improved muscle recruitment selectivity by non-invasive multielectrode tSCS may be possible.

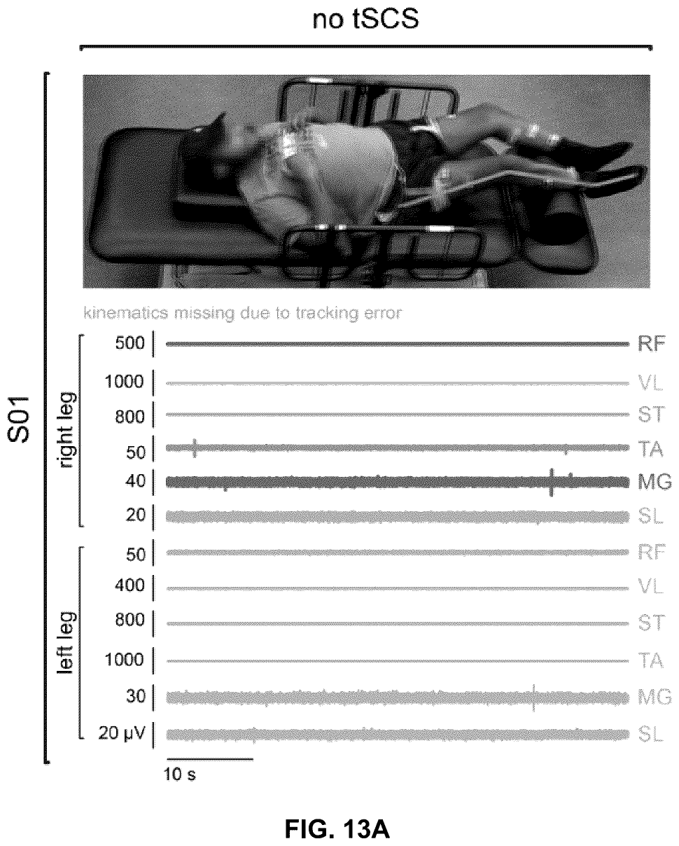

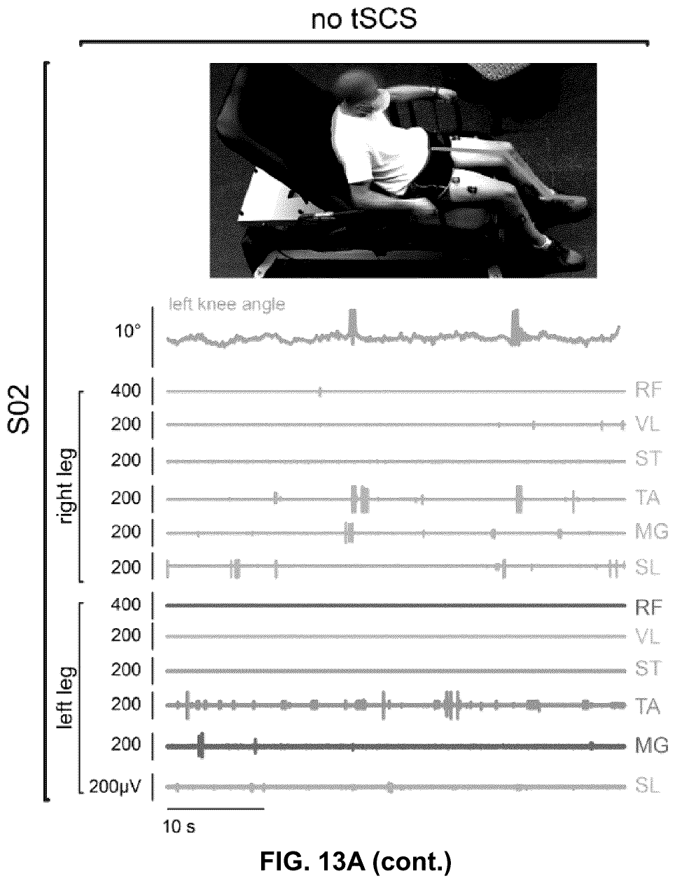

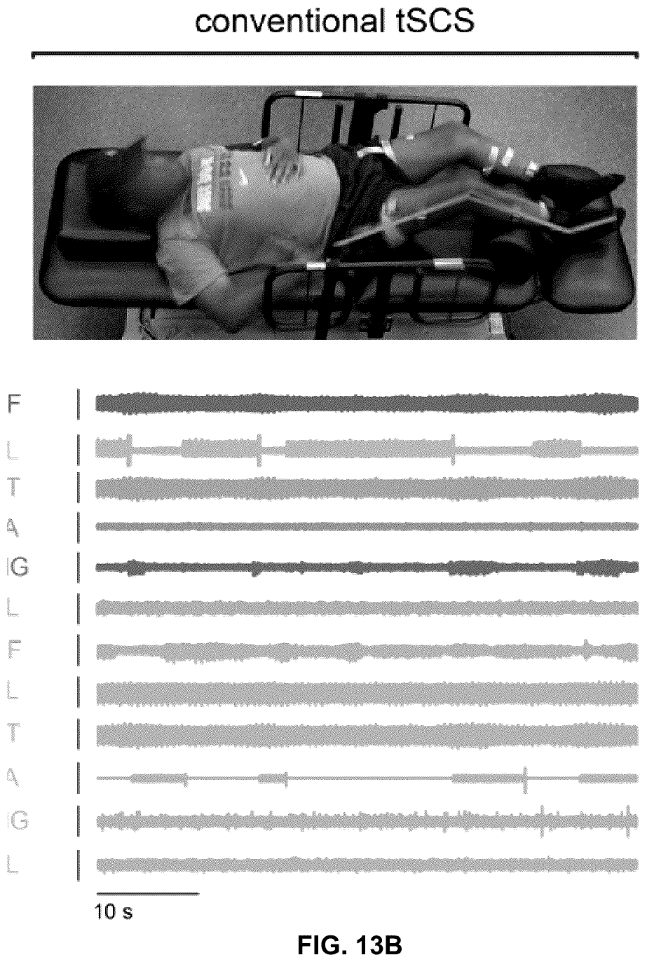

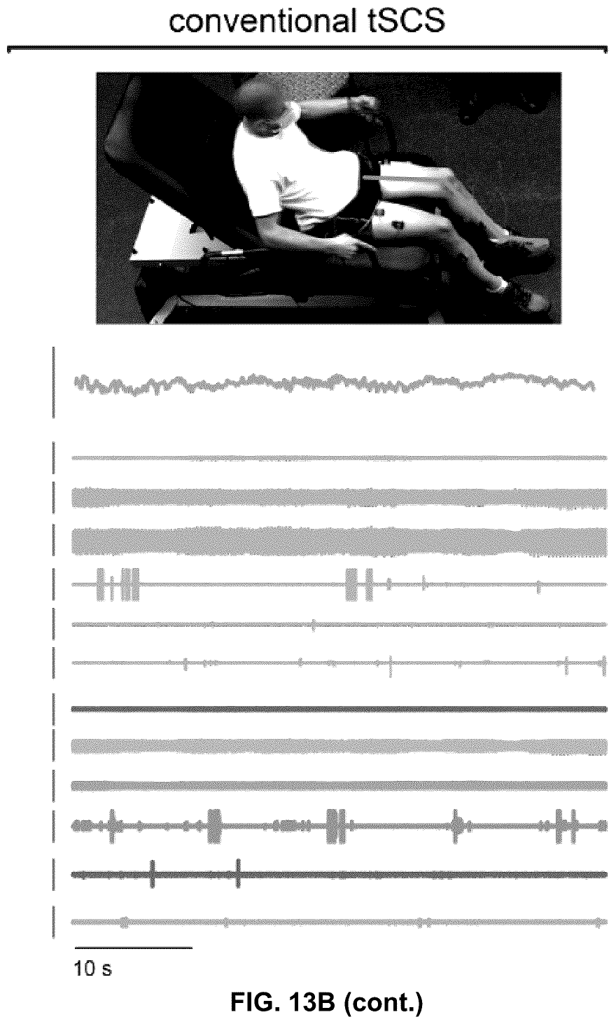

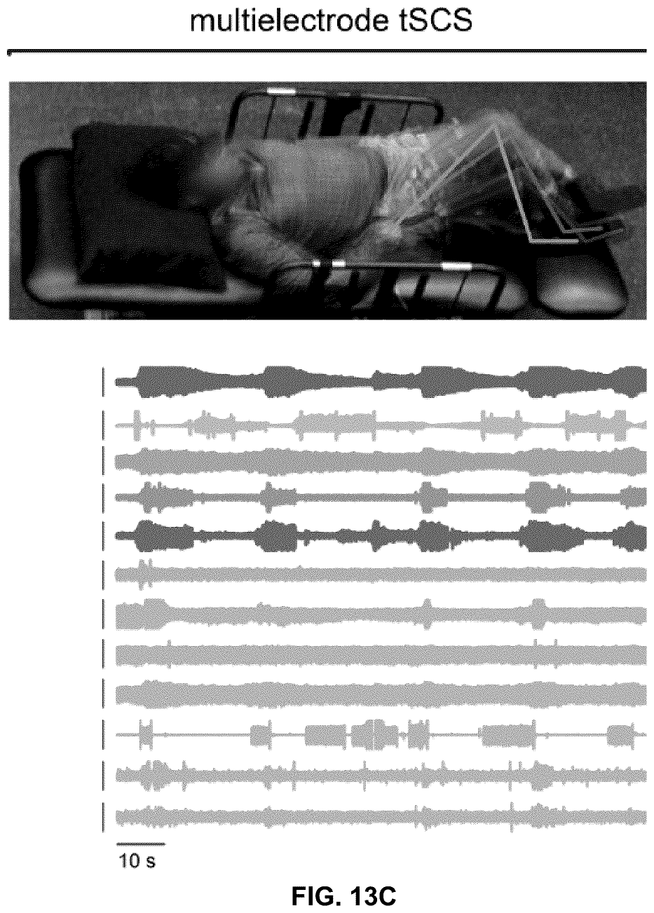

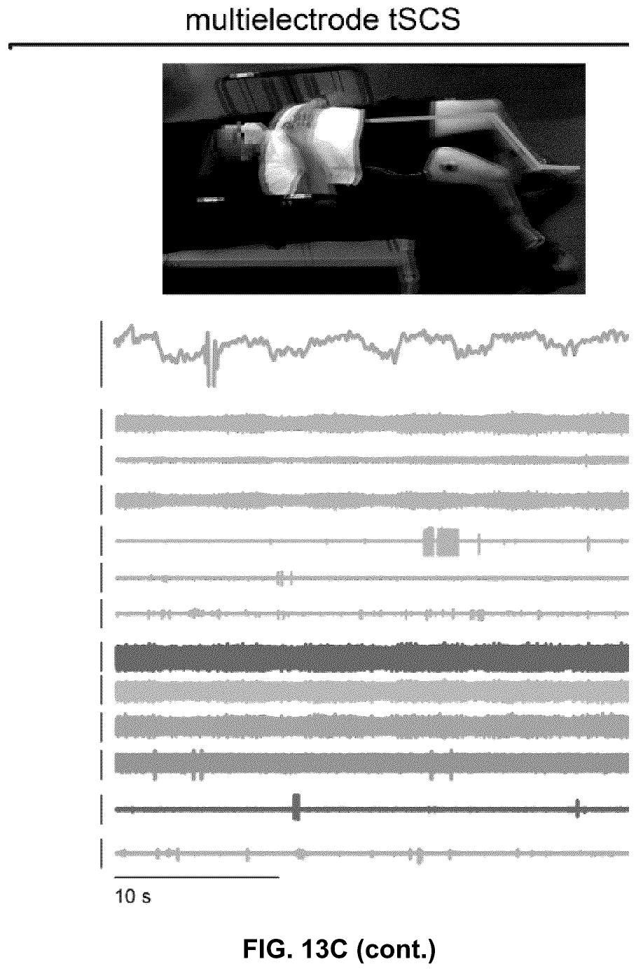

Recent findings have revealed that positioning a single tSCS surface electrode at different locations can enable the preferential recruitment of either muscles ipsilateral to the stimulation site or rostral vs. caudal muscle groups in the lumbosacral region. However, repositioning an electrode to target different muscle groups within a rehabilitation session would significantly limit the feasibility of performing this approach in a clinical setting. To address this limitation, we recently demonstrated that it is possible to have a single experimental setup in a multielectrode configuration that enables improved recruitment selectivity of proximal vs. distal muscles as well as muscles ipsilateral to the stimulation site compared to conventional tSCS. In the study, stimulation by the conventional tSCS electrode configuration resulted in broad non-selective muscle recruitment, as reflected by similar motor thresholds across rostral, caudal, and bilateral muscles (FIG. 13A). In contrast, motor thresholds were lower in a subset of muscles recruited by the right caudal electrode, and their recruitment level was close to saturation by the motor threshold amplitude of the others (FIG. 13B, note the differences in recruitment level at ˜40 mA).

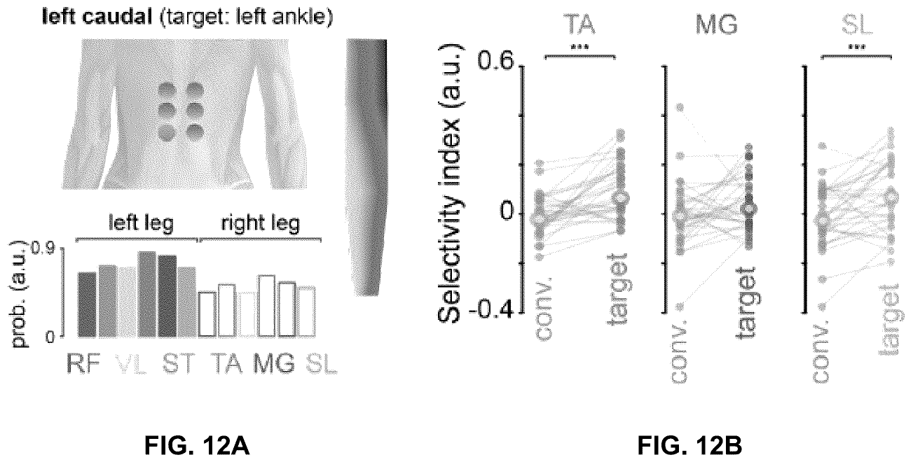

To identify the optimal electrode position for targeting each recorded leg muscle, the selectivity index for all muscles and electrode configurations across 16 neurologically intact participants was computed. Then, the electrode with the highest selectivity index for a particular muscle was selected as the target electrode for that muscle. The selectivity index varies from −1 to 1 and reflects the activation of a targeted muscle compared to the average activation of all other muscles, with a value of 0 indicating that a given muscle is recruited equally to the average recruitment of all other muscles. After identifying the optimal electrode to target each muscle, the hypothesis that the target ipsilateral electrode in the multielectrode configuration could enhance the recruitment selectivity of a targeted muscle group compared to conventional tSCS was tested. As shown in FIG. 12, the target electrode in the multielectrode configuration significantly enhanced recruitment selectivity compared to the conventional electrode for most muscles.

These improvements in muscle recruitment selectivity represent initial advances toward the long-term goal of applying spatial (where) and temporal (when) stimulation control of tSCS in clinical and community environments. However, although these improvements in muscle recruitment selectivity show promise, the ability of multielectrode tSCS to improve muscle activation patterns or enhance functional recovery in individuals with SCI has not yet been demonstrated. Additionally, the potential to further enhance selectivity by the preferential targeting of specific muscle groups using different stimulation frequencies remains largely unexplored.

While the study demonstrated improved muscle recruitment selectivity from single pulse responses, translating these findings into the continuous muscle activation patterns required to facilitate movements in people with SCI is a critical next step. To advance this research, it is crucial to conduct the next level of testing on individuals with SCI and impaired mobility, as they are the intended end users and the ones who would benefit the most from this technology. While improving muscle recruitment selectivity was one focus, the ultimate goal of rehabilitation is to enhance movement capacity. Given the unique nature of each SCI, the level of assistance required by individual muscle groups will vary. By employing multielectrode tSCS with selective targeting of specific muscle groups, one can achieve the desired activation in muscles that require assistance while avoiding the undesired activation of muscles that individuals still possess residual control over. This approach ensures a more tailored and effective rehabilitation strategy that respects the individuality of each person's level of function and optimizes their potential for functional recovery.

While experiments conducted on control participants are valuable for assessing single pulse responses, conducting these experiments with individuals with SCI is not only imperative but also enhances the feasibility of this approach for several reasons. First, due to a ceiling effect, motor-enhancing effects may be difficult to observe in individuals without movement deficits. In contrast, individuals with SCI offer an opportunity to observe significant improvements in motor function. Second, while only a limited number of unimpaired participants in the laboratory have tolerated continuous 30 Hz stimulation at motor threshold amplitudes, individuals with SCI, both in the laboratory of the inventors and studies by other groups, have demonstrated tolerance for continuous stimulation amplitudes at or above the motor threshold.

The motor-enabling effects of conventional and multielectrode tSCS were compared in two individuals with SCI (S01: C6-C7, incomplete; S02: T4-T6, incomplete). Although both participants self-reported their AIS level as AIS C, where some residual motor function below the injury is typically available, they were not able to voluntarily move any tested leg joint against gravity. When asked to move a single joint against gravity, neither participant was able to activate their leg muscles or produce movement (FIG. 13A). In contrast, stimulation at the motor threshold with conventional tSCS enabled some activation of leg muscles that was time-locked to their volition (FIG. 13B). This muscle activity was enough to produce a slight movement of the targeted joint. Moreover, the use of multielectrode tSCS targeting that joint further enhanced muscle activity and leg movements compared to conventional tSCS (FIG. 13C).

Although preliminary experiments showed that participants with SCI who could not produce unassisted voluntary leg movements against gravity were able to produce them with tSCS, they were exerting maximum volition, and the effects were admittedly limited. It is believed that tSCS holds a greater potential for augmenting residual motor function rather than enabling a new function that is not already available. To focus on individuals with SCI who have some level of residual motor function (AIS-C and D) to maximize the likelihood of observing a motor-enabling effect by multielectrode tSCS, the following experiments were conducted.

The rostrocaudal and unilateral organization of the leg muscles' motor neuron pools in the spinal cord, shown in FIG. 1A, facilitated the preferential targeting of proximal vs. caudal as well as ipsilateral vs. contralateral muscles using our multielectrode array. However, it is important to note that the electrode providing the optimal selectivity for a given muscle was always the same electrode that provided optimal selectivity for its antagonist muscle. For instance, the bottom electrode is optimal for the ipsilateral ankle flexors (tibialis anterior) and extensors (medial gastrocnemius and soleus) (FIG. 12). In other words, although spatial stimulation control enables unilateral assistance to either the hip or ankle joints, discriminating between flexion and extension assistance, and thus the potential for co-contractions remained a challenge. This observation may be attributed, in part, to the significant overlap in the locations of posterior roots for both flexor and extensor muscles in the lumbosacral spinal cord (FIG. 1A). Notably, simultaneous recruitment of agonist and antagonist muscles occurs also in eSCS, and various stimulation approaches, such as multipolar configurations and stimulation frequency modulation have been employed to address this challenge. Developing stimulation strategies that go beyond spatial control to improve muscle recruitment selectivity could overcome a critical barrier in non-invasive technologies, enabling the execution of dexterous movements using tSCS.

In previous work in eSCS, the stimulation frequencies were adjusted to tune muscle activity during gait. As observed in animal models, increasing the stimulation frequency from 20-100 Hz proportionally increased flexor muscle activity. In contrast, extensor muscle activity was inversely related to stimulation frequency, such that the highest extensor activity occurred at stimulation frequencies below 30 Hz. These results confirmed previous neurophysiological observations by other groups showing a frequency-dependent selection of alternative spinal pathways to generate rhythmic or sustained movements. While proprioceptive afferents predominantly activate flexor motor neurons through polysynaptic circuits, they elicit robust monosynaptic responses in extensor motor neurons. These differences in frequency-dependent modulation have therefore been attributed to differences in presynaptic mechanisms between flexor and extensor muscles, where low-frequency SCS reveals primarily monosynaptic responses, while high-frequency SCS induces long-latency polysynaptic responses. Although frequency modulation presents a promising approach to selectively targeting flexor vs. extensor muscles, to our knowledge, it has not been tested in non-invasive tSCS. If successful, stimulation by our multielectrode tSCS array targeting the ankle muscles could be further improved to selectively activate either the tibialis anterior or the medial gastrocnemius and soleus muscles, and a similar approach could be employed at the hip and knee joints. This novel approach opens possibilities to improve the types of exercises that can be practiced during tSCS-assisted rehabilitation.

Besides enhancing the immediate prosthetic effect enabled by tSCS, several questions remain to establish the feasibility of translating multielectrode configurations of tSCS into clinical practice for long-term rehabilitation. As rehabilitation researchers with lived experiences in neuromotor disorders have pointed out, “we often overlook qualities of robustness, expense, simplicity, and setup time in order to push the limits of technology”. Undoubtedly, incorporating multiple electrodes that can be controlled with different frequencies increases the complexity of the hardware and software requirements for tSCS. Although these technological advancements in tSCS may not differ significantly from commercially available devices for multielectrode functional electrical stimulation (FES), the feasibility of this approach must be thoroughly evaluated.

The feasibility of multielectrode tSCS at an adaptive community-based exercise center will be systematically assessed to identify potential implementation challenges, and develop strategies to overcome them, ultimately optimizing its use in the context of rehabilitation. We will investigate key important factors, including the setup time required, the amount of effective training time possible within a typical 1-hour session, the frequency of exercise interruptions, the level of active participation facilitated by the system, and the robustness of the parameters when transitioning from controlled laboratory settings to real-world community environments are investigated. By addressing these essential questions, the study smooths the translational pathway for employing spatial and frequency tSCS control in clinical and community settings.

The objective of this study is to develop a personalized optimization protocol that can be used to evaluate the effect of different multielectrode tSCS parameters on the leg motor function of individuals with SCI in an evidence-based manner. The goal is to create an efficient and translatable procedure that can be readily applied in a community setting. It is hypothesized that multielectrode tSCS can lead to a higher level of leg motor function by preferentially activating leg muscle motor neuron pools through sensory afferents. To establish the feasibility of translating multielectrode tSCS into a rehabilitation intervention, changes in leg motor function enabled by spatial and frequency control of tSCS in 8 people with chronic SCI (>1-year post-injury) and at least some amount of residual leg motor function (AIS C and D) is characterized. In a pilot clinical trial on an additional group of 8 SCI participants (AIS C and D, >1-year post-injury), facilitators and barriers to translating this approach into an activity-based training platform in a community setting are identified. The evidence-based knowledge generated by this study informs the optimization of a future clinical trial to evaluate the clinical efficacy of multielectrode tSCS-assisted rehabilitation in people with SCI.

Transcutaneous SCS is a low-cost, non-invasive method that has the potential to be set up by individuals with SCI with the help of a caregiver or personal care attendant and be incorporated into community-based exercise centers to enhance traditional exercise. The community partner, the Orthwein Center, is an ideal collaborator for the planning and implementation of our feasibility study with their trained health professional staff (e.g., physical therapists, occupational therapists, occupational therapist assistants, physical therapist assistants, etc.) and accessible equipment (wheelchair treadmills, body-weight supported treadmill, exoskeletons, Lokomat, FES cycle, etc.). Individuals with SCI who use these centers have typically undergone rehabilitation and have a firsthand understanding of the barriers associated with accessing such interventions during rehabilitation. Furthermore, as the participants are no longer receiving rehabilitation, the study does not interfere with ongoing therapy or the potential recovery process. Given the abundance of devices for functional electrical stimulation and tSCS, the objective is not to engineer a novel device. Rather, a comprehensive testing paradigm enabling researchers to assess the impact of varying stimulation parameters on motor function within both laboratory and clinical environments is developed and made publicly available. The goal is to identify potential hurdles that may limit the translation of this approach to clinical and community settings, where the personalized optimization of SCS can most effectively respond to individual differences in injury, residual abilities, and unique impairments.

Given the unique nature of each SCI, individualized levels of assistance may be essential. Rather than delivering non-selective stimulation to all leg muscles, multielectrode tSCS can provide targeted assistance only to those muscles that require it, thus maximizing the function of residual circuitry toward improved recovery.

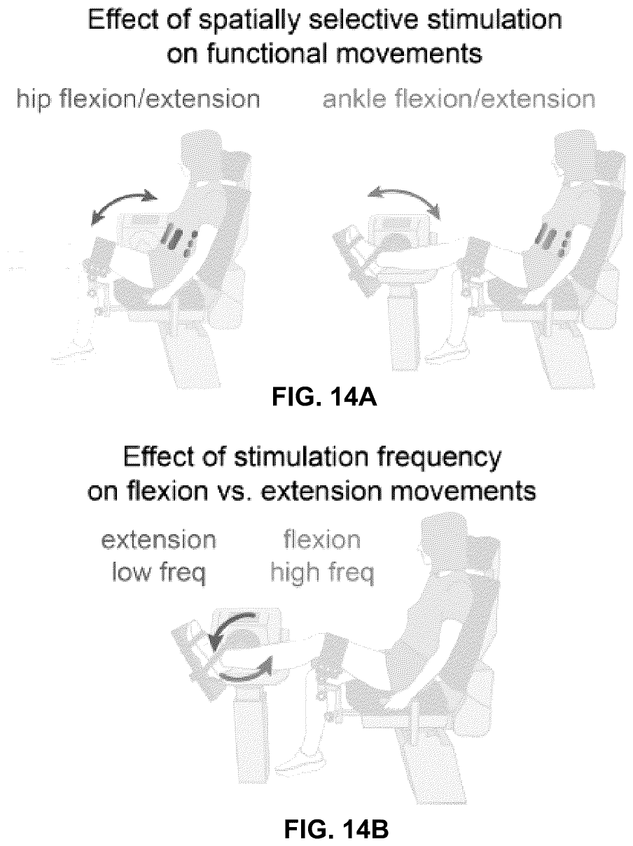

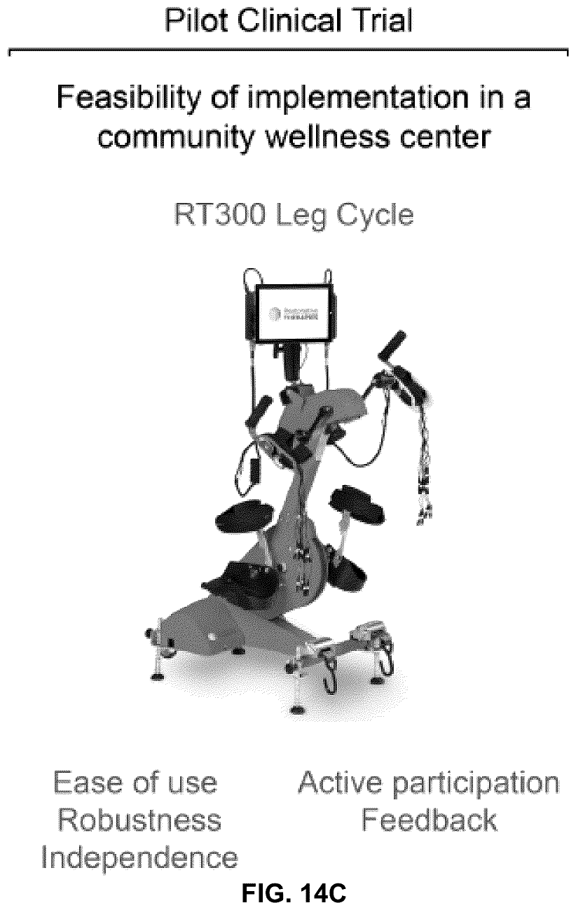

An initial neurophysiological evaluation verifies the types of responses elicited by conventional and multielectrode tSCS in each participant. The hip, knee, and ankle joints are evaluated in three separate 3-hr sessions where participants use their leg movements to perform a torque-control task in an isokinetic dynamometer (FIG. 14A). Three conditions are tested in each session: (1) using their residual voluntary leg movements (no stim), (2) using a single large electrode (conventional tSCS), and (3) using the target electrode in the multielectrode array (multielectrode tSCS). The motor-enabling effect is evaluated by measuring the torque production enabled by each stimulation configuration. Joints are tested bilaterally and in randomized order. In three additional 3-hour sessions, participants perform the same task for each joint separately under different stimulation frequencies (FIG. 14B). There is at least 1 day between sessions to allow time for recovery and stimulation washout.

Participants are asked to produce isometric torque movements at the hip, knee, and ankle joints under the no stim, conventional tSCS, and multielectrode tSCS conditions. Participants sit comfortably on an isokinetic dynamometer (System 4 Pro, Biodex) and are instructed to follow a sequence of instructions on the screen: prepare to move at an auditory cue, move at a go cue, and finish the movement at an end cue. To evaluate the participant's maximum isometric torque generation at a given joint, the dynamometer's position is locked at a resting, slightly flexed position (hip, 120°; knee, 160°; ankle, 110° plantar flexion).

Participants are asked to perform 5 repetitions of maximum voluntary contraction under each stimulation condition. This entails 15 repetitions of the same movement per session, and a total of 60 bilateral movement attempts in a 3-hour session (5 repetitions×2 movement types (flexion/extension)×3 conditions (no stim, conventional tSCS, multielectrode tSCS)×2 legs (right/left)). Further, participants are asked to perform 5 repetitions of maximum voluntary contraction under 11 stimulation frequency conditions (5, 10, 20, 30, 40, 50, 60, 70, 80, 90, and 100 Hz). Only one leg and joint are tested, with a total of 55 repetitions of the same movement in a 3-hour session, and a total of 110 unilateral movement attempts (5 repetitions×2 movement types (flexion/extension)×11 frequency conditions). Conditions are tested in randomized order, and joints are tested in separate sessions. Participants are allowed to rest as much as needed, with a minimum resting period of 2 minutes between trials. If signs of fatigue in torque and EMG performance are observed, individual sessions are split into two days.

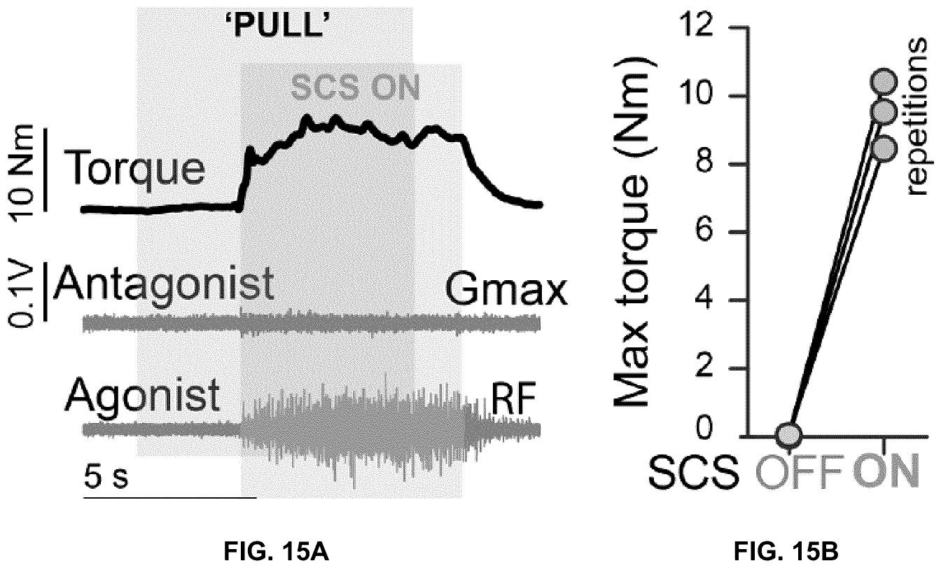

During the stimulation conditions of the study, leg movements are reinforced (FIG. 15) by delivering non-invasive electrical stimulation to the lumbar spinal cord. The degree of recruitment of each muscle with increasing stimulation amplitudes (recruitment curves) is computed by measuring graded amplitude recordings as participants lay supine on a table. Leg movements during the stimulation phases of the study are reinforced by delivering tSCS to the lumbar spinal cord using biphasic, rectangular, 1 ms pulses at a frequency of 5-100 Hz and an amplitude of 10-200 mA with a non-invasive multichannel ARC-Ex stimulator (ONWARD Medical, NL). In the conventional tSCS condition, stimulation is using a 5×9 cm conductive self-adhesive electrode placed over the spinous processes of the T11/T12 vertebrae and two 7.5×10 cm electrodes over the abdomen. In the multielectrode condition, six 3.2 cm diameter round electrodes lateral to the midline and centered at the T11/T12 interspinous ligament are positioned, and only the abdominal electrode ipsilateral to the target electrode is used as the return electrode.

Kinematic, isokinetic (joint torque, angle, and velocity), and electromyographic (EMG) recordings are obtained using a 10 HD markerless camera system (Miqus-Hybrid, Qualisys, Sweden) with a deep learning algorithm for rigid body estimation (Theia Markerless, Canada), the isokinetic dynamometer, and a 16-channel wireless EMG system at 4370 Hz (Trigno Avanti, Delsys, Natick, MA) respectively, and processed. Hip, knee, and ankle joint torques, together with EMG activity in the iliopsoas, rectus femoris, vastus lateralis, semitendinosus, tibialis anterior, medial gastrocnemius, and soleus muscles, allows the characterization (FIG. 15A) and quantification (FIG. 15B) of the effect of SCS on muscle activity and movement capacity throughout the experiment. Kinematic recordings are used to verify that no compensatory strategies are being used.

Additionally, a pilot clinical trial is conducted to evaluate the feasibility of applying multielectrode tSCS in an adaptive community-based exercise center. This pilot clinical trial enrolls 8 additional participants with SCI. In a 2-day tSCS spatial and frequency optimization session, recruitment curves in the multielectrode tSCS configuration are generated, followed by torque generation evaluations on the isokinetic dynamometer. The objective of this session is to identify the optimal electrode for hip and knee targeting (day 1) as well as the optimal frequency for flexion vs. extension movements (day 2). The volunteer and an Adaptive Exercise Specialist then participate in a 1-day education session by Seanez Lab members at the Orthwein Center to learn and practice how to set up the electrodes and stimulation parameters on the ergometer bike. Because stimulation parameters vary, this education session is performed for each participant.

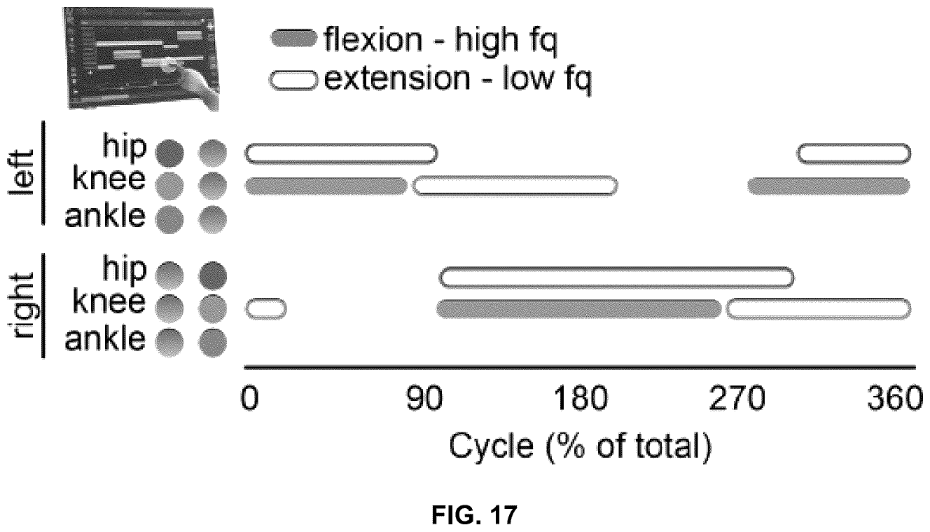

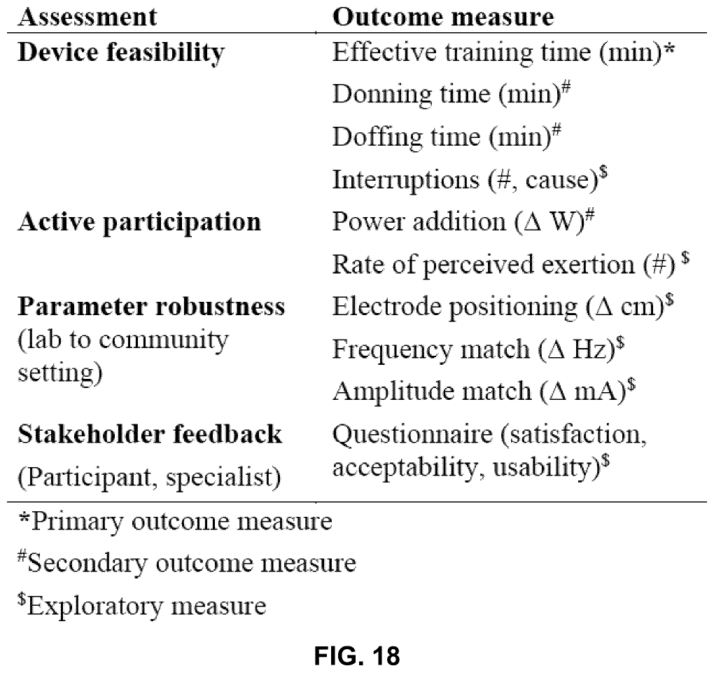

After stimulation patterns that enable hip and knee flexion and extension are identified, participants perform five 1-hour sessions of tSCS-assisted leg cycling on a stationary leg ergometer bike at the Orthwein Center, with the help of the Adaptive Exercise Specialist. An open-loop, pre-programmed stimulation protocol is configured to alternate between hip and knee flexor and extensor movements (FIG. 17). Similar stimulation patterns have been shown to increase power generation in FES cycling. Stimulation amplitudes between “quiet” and SCS periods have a short ramping period between 0.7× the motor threshold and the effective amplitude, respectively. This approach is intended to prevent discomfort from sudden, large increases in stimulation amplitude. The specialist empirically adjusts the stimulation parameters (burst duration, amplitudes, and frequencies) according to the participant's feedback. Two additional researchers are present during all training sessions for technological assistance and data collection for outcome measures, respectively.

Several metrics may characterize technological aspects that impact the usability of our proposed multielectrode tSCS approach in a community environment. Because managing the added complexity of multielectrode tSCS within the limited therapy time will be one of the main challenges in a rehabilitation setting, the primary outcome measure will be the total time participants spend cycling during a 1-hour session (effective training time). A clinically feasible time cost that would foster the implementation of new technology into routine clinical rehabilitation practice has been estimated to be less than 13.5 minutes in a 1-hour session. This suggests that for the technology to be feasible, donning, doffing, and interruptions should still allow for 46.5 minutes of effective training time within the 1-hour session. Considering the device is still in the early development phase and is not a commercial product, an effective training time greater than 40 minutes within a 1-hr session is targeted. This critical metric is carefully recorded by a designated data collector and cross-verified using the devices' logged training time. Unlimited resting breaks are allowed as the participant and therapist see fit, and the stopwatch is paused until they resume, to avoid time pressure on resting.

Other measures include the donning and doffing times, which play crucial roles in their adoption by therapists and people with neuromotor disorders into their practice and exercise routine, respectively. Additionally, the ergometer's cycling power that is added during multielectrode tSCS relative to a no-stimulation condition (recorded during the education session day) may be assessed. Prior studies have documented increased efficiency of FES-based cycling approaches with up to 33% additions in power.

Other measures include assessing the number of interruptions during training and identifying their causes, gauging the rate of perceived exertion, examining differences in stimulation parameters between the laboratory and training sessions, and administering a device questionnaire to gather valuable feedback from both participants and therapists. By analyzing these diverse outcome measures, comprehensive insights into the effectiveness, feasibility, and user experience of multielectrode tSCS-assisted cycling for individuals with spinal cord injury in community and rehabilitation environments are gained.

Targeting People with Residual Motor Functions to Maximize the Probability of Early Success.

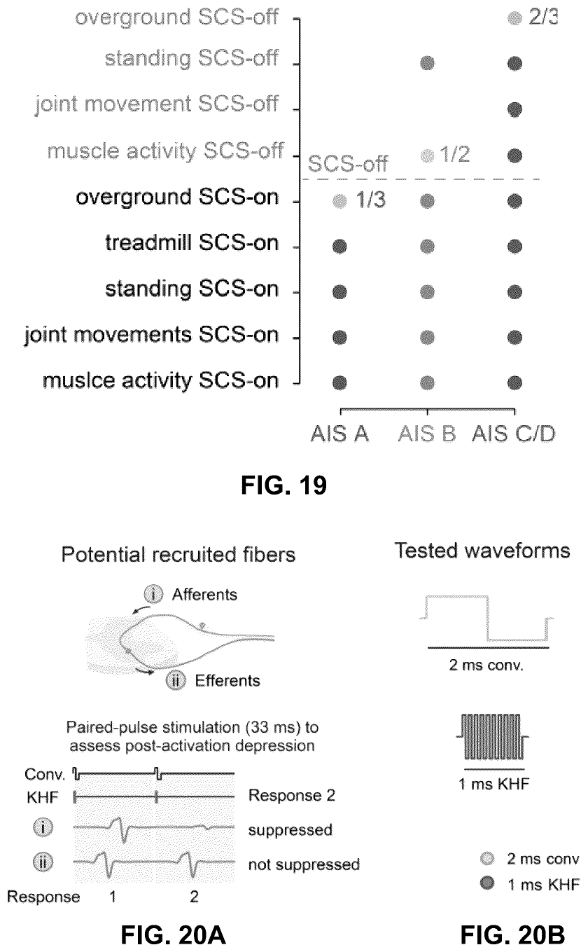

Results from early studies seemed to indicate that recovery outcomes by SCS-assisted long-term rehabilitation (>5 months to a year) correlate with lesion severity at study enrollment (FIG. 19). Promisingly, results from a recent multi-center, large-scale clinical trial using tSCS on 60 individuals with SCI showed that although participants with AIS-C and AIS-D were indeed more likely to be responders (˜90% and ˜60% responders, respectively), there was still a significant portion of the population with AIS-B to show improvements in function and/or strength (˜45%). In this study, it is not aimed to restore motor function by long-term training. Instead, recruitment mechanisms and the immediate effects of different tSCS parameters on motor function are characterized. Therefore, this pilot clinical trial was designed to maximize the probability of successfully observing a measurable effect while minimizing variability across participants. The inclusion criteria require participants to have a minimum motor score of 1 on at least two muscles (AIS-C/D) to ensure data collection on joints with some residual capacity. However, many participants will likely have no residual motor function on some joints. This will provide crucial control data on the ability of tSCS to enhance motor function on joints without residual motor function, at least within participants. This knowledge is then used to optimize the approach to best target not only those with residual motor function (AIS-C/D) in future clinical trials but also those with more limited motor capacity (AIS-A/B).

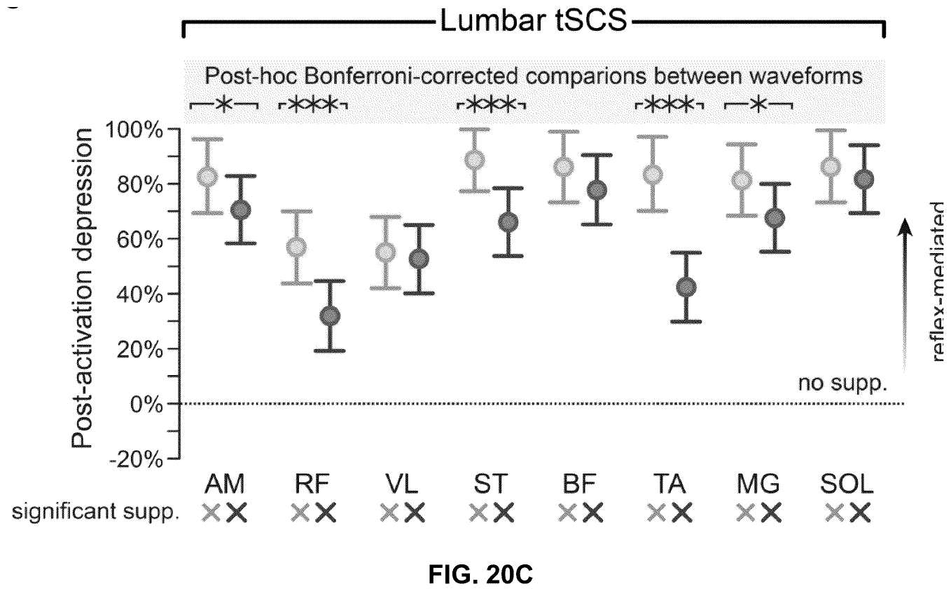

In an initial neurophysiological evaluation, the degree of muscle recruitment (response amplitude) with increasing stimulation amplitudes is computed (FIGS. 1 and 2). Moreover, it is verified that responses elicited by conventional and multielectrode tSCS are mediated by posterior root-muscle reflexes using a double-pulse stimulation paradigm (FIG. 20). Due to the long time required to complete full recruitment curves for every electrode, only motor thresholds will be collected for each muscle at subsequent sessions.

During the stimulation conditions of the study, leg movements are reinforced by delivering non-invasive electrical stimulation to the lumbar spinal cord using biphasic, rectangular, 1 ms pulses at a frequency of 30 Hz and an amplitude of 10-200 mA78 with a DS8R non-invasive, current-controlled, biphasic stimulator (Digitimer, UK) (FIG. 13). In the conventional tSCS condition, stimulation is administered using 5×9 cm conductive self-adhesive electrode placed over the spinous processes of the T11/T12 vertebrae and two 7.5×10 cm electrodes over the iliac crests. In the multielectrode condition, six 3.2 cm diameter round electrodes are positioned lateral to the midline and centered at the T11/T12 interspinous ligament, and only the abdominal electrode ipsilateral to the target electrode is used as the return electrode 10. All tSCS electrodes are commercially available PALS Neurostimulation Electrodes (Axelgaard Manufacturing Co., Ltd, USA).

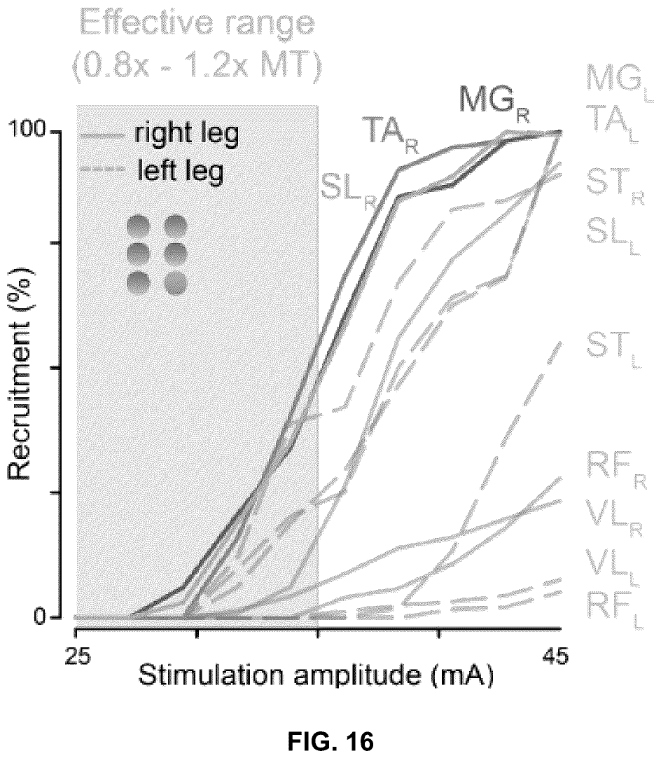

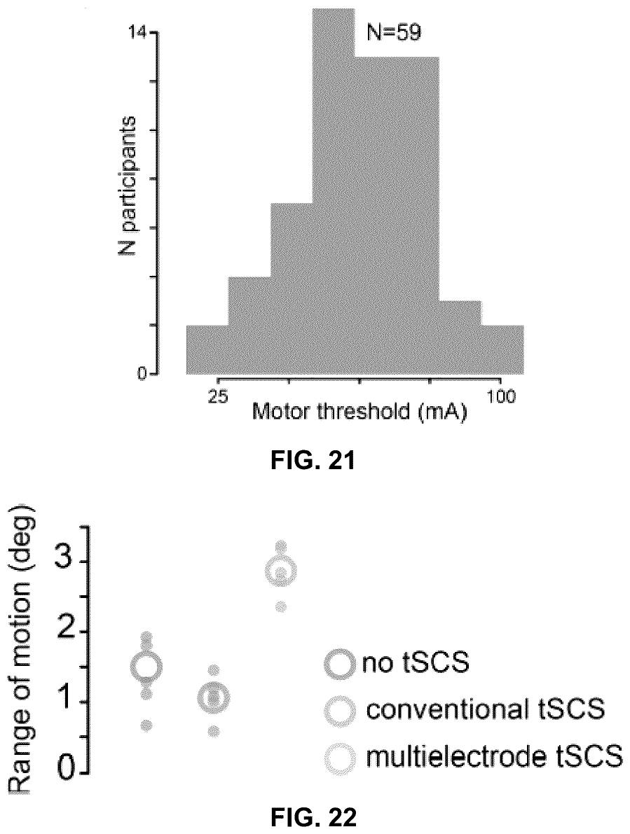

Stimulation intensity is empirically adjusted to the maximum tolerable stimulation amplitude with a motor-enabling effect or 1.2× the motor threshold, whichever is lower (effective amplitude, FIG. 16). The distribution of motor threshold values collected in our laboratory from 59 unimpaired participants is shown in FIG. 21. Because neural circuits below the injury in people with SCI remain dormant but functionally intact, it is expected these values to be similar or slightly higher. To allow participants to habituate to the stimulation and prevent discomfort, the stimulation amplitude is slowly increased until the effective amplitude at the beginning of each condition.

Power analysis is based on preliminary SCI data (FIG. 22) using a conservative within-subject effect size (Cohen's d=0.8). A sample size of 16 participants provides >80% power at α=0.05 to detect large treatment effects. The repeated-measures crossover design yields high statistical efficiency. Each participant contributes approximately 180 repeated torque and EMG observations across three conditions, five sessions, and twelve muscles, resulting in ˜2,880 observations in total. This high-density structure improves precision and reduces the influence of modest missingness.

### Example 8—Determine the Effect of Stimulation Frequency on the Recruitment of Flexor and Extensor Muscles

### Introduction and Rationale

Previous studies indicate that frequency modulation can be exploited to selectively target the recruitment of flexor or extensor muscles. However, these have only been reported during invasive eSCS, and the effect of stimulation frequency on the recruitment of both flexor and extensor leg muscles in tSCS remains unknown. The effect on recruited neural circuits by different stimulation frequencies in tSCS in individuals with SCI was evaluated, and tSCS stimulation strategies incorporating the modulation of stimulation frequency to maximize the motor effect during flexion and extension tasks were developed.

Neuroanatomical Basis for Improvements and Limitations in Leg Muscle Recruitment Selectivity by Multielectrode tSCS.

The rostrocaudal and unilateral organization of the leg muscles' motor neuron pools in the spinal cord (FIG. 1A) facilitated the preferential targeting of proximal vs. distal as well as ipsilateral vs. contralateral muscles using the described multielectrode array. The rostrocaudal and unilateral organization of leg muscles' motor neuron pools in the spinal cord enables the selective recruitment of individual muscle groups by small-diameter electrodes centered at the T11/T12 vertebrae. However, it is important to note that the electrode providing the optimal selectivity for a given muscle was always the same electrode that provided optimal selectivity for its antagonist muscle. For instance, the T12/L1 electrode is optimal for the ipsilateral ankle flexors (tibialis anterior) and extensors (medial gastrocnemius and soleus) (FIGS. 1H, 2C, and 2D). In other words, although spatial stimulation control enables unilateral assistance to either the hip or ankle joints, discriminating between flexion and extension assistance, and thus the potential for co-contractions remains a significant challenge. This observation may be attributed, in part, to the significant overlap in the locations of posterior roots for both flexor and extensor muscles in the lumbosacral spinal cord (FIG. 1A). Developing stimulation strategies that go beyond spatial control to improve muscle recruitment selectivity could overcome a critical barrier in non-invasive technologies to enable the execution of dexterous movements. Notably, simultaneous recruitment of agonist and antagonist muscles also occurs in eSCS, and various stimulation approaches, such as multipolar configurations and frequency modulation, have been employed to address this challenge. The potential to further enhance selectivity by the preferential targeting of flexor vs extensor muscles in tSCS needs to be explored.

### Frequency Modulation for Selective Activation of Flexor and Extensor Muscles.

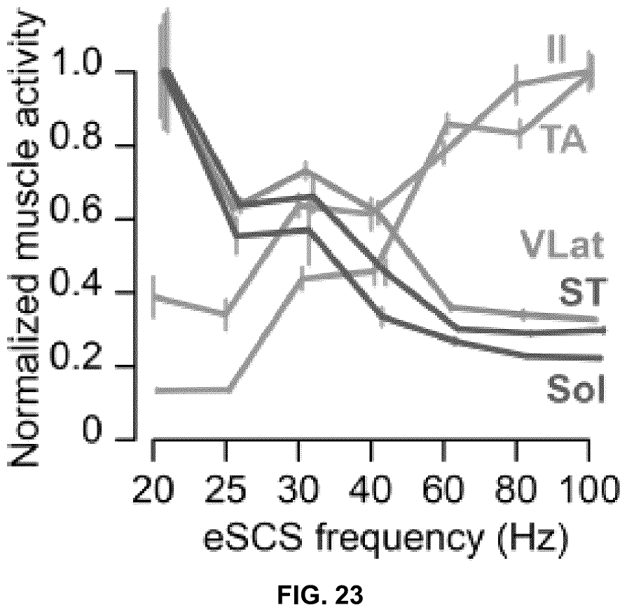

Two decades ago, neurophysiological observations by other groups showed a frequency-dependent selection of mono- or poly-synaptic spinal pathways in eSCS. While proprioceptive afferents predominantly activate motor neurons through polysynaptic circuits in flexor muscles, they elicit robust monosynaptic responses in extensor muscles. Therefore, it was hypothesized that it should be possible to use different stimulation frequencies to preferentially recruit flexor vs. extensor muscles. In previous work in eSCS, the stimulation frequencies were adjusted to tune muscle activity during gait. As observed in animal models, stimulation frequencies from 40-100 Hz proportionally increased flexor muscle activity, while frequencies from 20-40 Hz increased activity of extensor muscles in humans with SCI (FIG. 23). Although frequency modulation presents a promising approach to selectively targeting flexor vs. extensor muscles, it has not been tested in non-invasive tSCS. In some respects, stimulation by the multielectrode tSCS array targeting the ankle muscles can be further improved to selectively activate either flexor (tibialis anterior) or extensor (medial gastrocnemius and soleus) muscles, and a similar approach could be employed at the hip and knee.

In a cross-over study design, changes in muscle recruitment and voluntary torque production at the hip, knee, and ankle joints enabled by stimulation frequencies ranging from 5-100 Hz over 15 sessions are tested.

### Muscle Recruitment

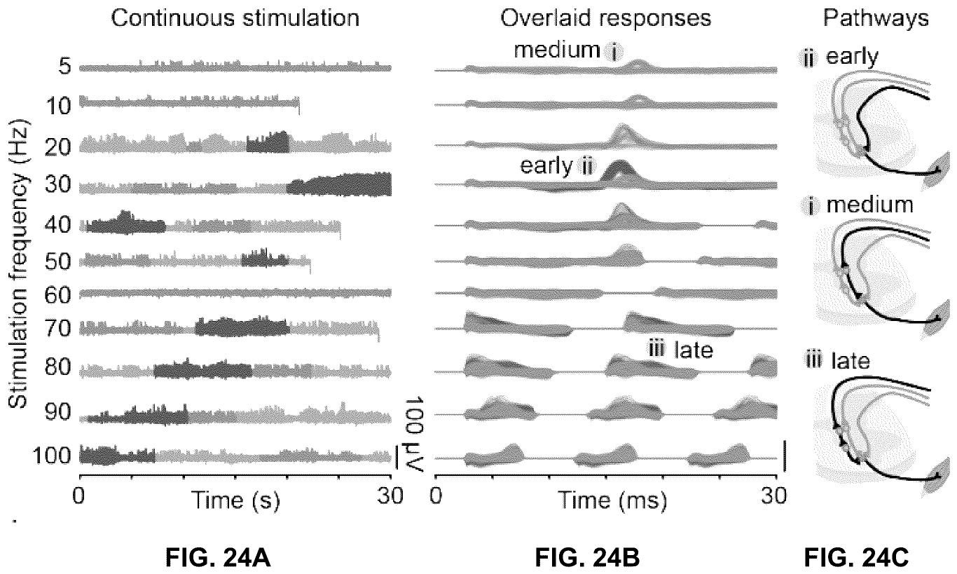

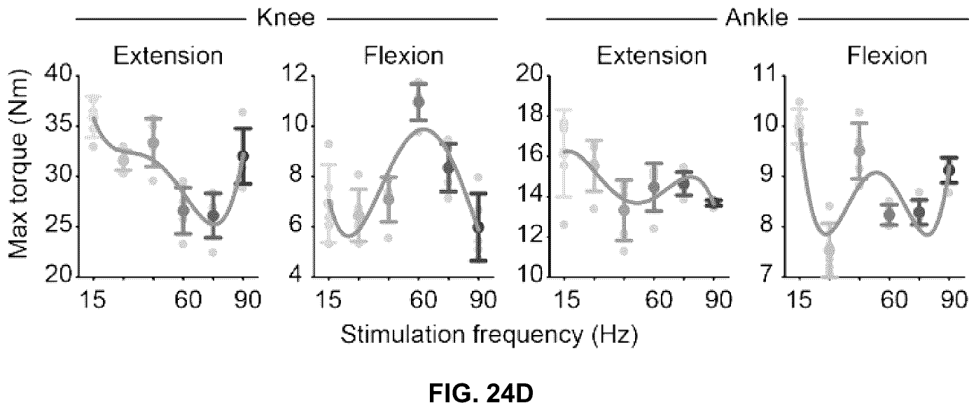

Leg muscle responses are recorded during rest to continuous tSCS at 6 stimulation frequency conditions (15, 30, 45, 60, 75, and 90 Hz) (FIG. 24A). The stimulation amplitude at 30 Hz slowly increased from 0 mA until the motor threshold to allow for adaptation to the stimulation-induced effects. Stimulation frequency is then varied to perform 30-second recordings at each frequency during rest.

### Motor Task

Participants are asked to perform 5 repetitions of maximum voluntary contraction under the 6 stimulation frequency conditions using the isokinetic dynamometer (FIG. 24D). Only one joint is tested each session, with a total of 110 movement attempts (5 repetitions×2 movement types (flexion/extension)×7 frequency conditions) per leg within a 3-hour session. Conditions are tested in randomized order, and joints are tested in separate sessions. Participants are allowed to rest between trials and sessions.

### Data Analysis

Linear mixed-effects models are used to assess the effect of stimulation frequency (15-90 Hz), movement type (flexion vs. extension), and their interaction on the primary outcome, voluntary torque production (FIG. 24D). Secondary outcomes include EMG response latency, amplitude, and waveform classification. Triggered EMG responses are segmented and analyzed separately for each stimulation frequency using linear mixed-effects models. We will employ a K-means clustering algorithm that is employed to group responses based on key electrophysiological features such as response onset latency, peak amplitude, and waveform shape. Covariates, missing data handling, and power assumptions follow a similar rationale as described above in previous examples.

By primarily recruiting agonist muscles without their agonists, frequency-dependent selection of flexor vs. extensor muscles can enable maximum muscle activity and torque capacity at low frequencies (50 Hz) for flexor muscles, as well as that low frequencies (50 Hz) can preferentially recruit muscles through polysynaptic pathways. This is reflected in the generation of short-to-medium response latencies with low frequencies for extensor muscles and medium-to-long response latencies with high frequencies for flexor muscles.

### Example 9—Determine the Effect of TSCS Amplitude Control on Muscle Recruitment, Motor Function, and Pain

### Introduction

Alternations in active muscle groups during different phases of movement require sudden changes in stimulation amplitude from the “off” to the “on” condition. However, sudden, large changes in stimulation amplitude have been qualitatively described as uncomfortable and are typically avoided by a slow ramp-up of stimulation amplitude. Preliminary experiments on unimpaired participants suggest that pre-conditioning the stimulation amplitude to a level just below the “effective” threshold allows for rapid adjustments in stimulation amplitude with minimal discomfort. To evaluate how changes in stimulation amplitude impact the recruitment of neural circuits, motor function, and pain, the following experiments were conducted.

Proportional Stimulation Control to Improve Comfort During Spatiotemporal tSCS.

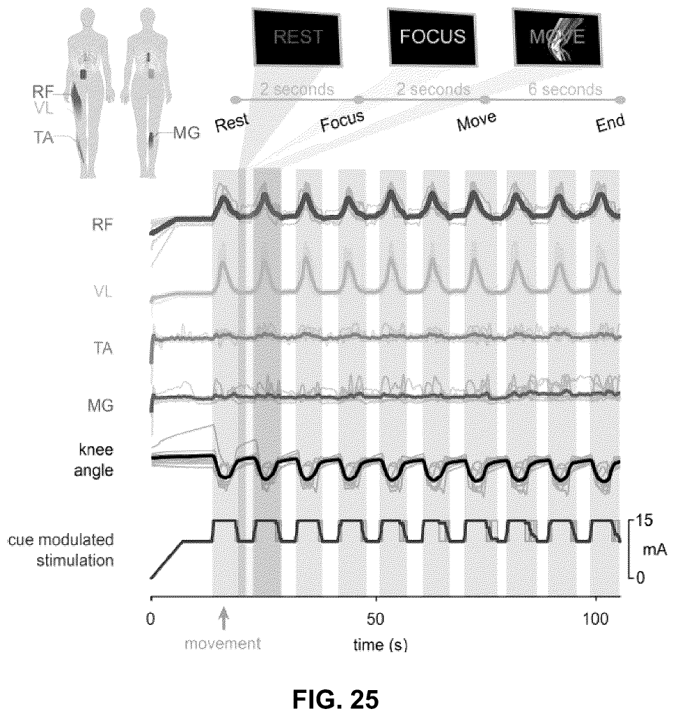

In spatiotemporal eSCS, electrodes are only active during the part of the movement where muscles targeted by those electrodes should be active (FIG. 1G). Due to the low stimulation amplitudes required (1-16 mA) and recruitment of large-diameter proprioceptive afferents, alternating between ‘off’ and ‘on’ amplitudes at each electrode is feasible in eSCS. In contrast, sudden alternations between ‘off’ and ‘on’ amplitudes are unlikely to be tolerated in tSCS due to the intense cutaneous and neuromuscular co-activation beneath the stimulating surface electrodes and the high stimulation amplitudes required (10-200 mA). In preliminary experiments on unimpaired participants, an alternative approach has been found that can increase tolerance in alternations between stimulation amplitudes. In the approach, stimulation amplitude is gradually increased at the beginning of the session to an amplitude close to, but below the “effective amplitude” and increased to the “effective amplitude” only during the movement phase (FIG. 25). This approach ensures that sudden jumps in stimulation amplitude are smaller and, therefore, more tolerable.

Stimulation amplitudes used for rehabilitation are empirically tuned based on therapist observations and participant feedback, and these intensities are typically reported in terms of milliamps. However, the stimulation amplitude that is required to enable a neuroprosthetic effect by tSCS, and how this relates to motor threshold or level impairment, remains unknown. Moreover, the amount by which stimulation amplitude can be increased without causing discomfort or the speed at which it can be increased remains poorly understood. In a cross-over study design, changes in muscle recruitment, motor function, and pain elicited by different stimulation intensities over 15 sessions are tested.

### Muscle Recruitment.

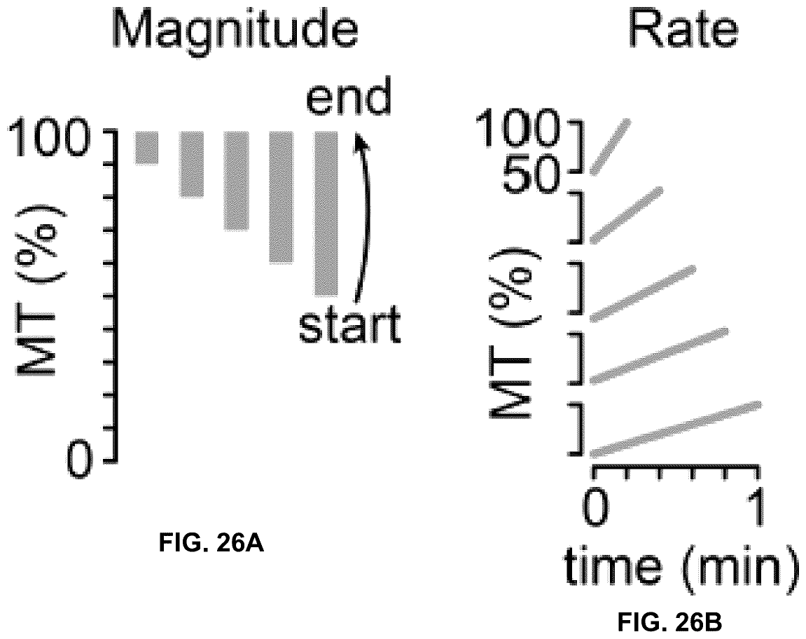

In an initial evaluation, leg muscle responses are recorded during the acquisition of recruitment curves using single pulse stimulation and during continuous 30 Hz stimulation for 30 seconds at different stimulation intensities (50%-120% motor threshold). In additional sessions, the effect of changes in stimulation magnitude (FIG. 26A), rate of change (FIG. 26B), and the combination of both on muscle recruitment, motor performance, and pain are investigated. Changes in stimulation amplitude and their relation to objective and subjective quantifications of pain are evaluated during rest and voluntary torque control.

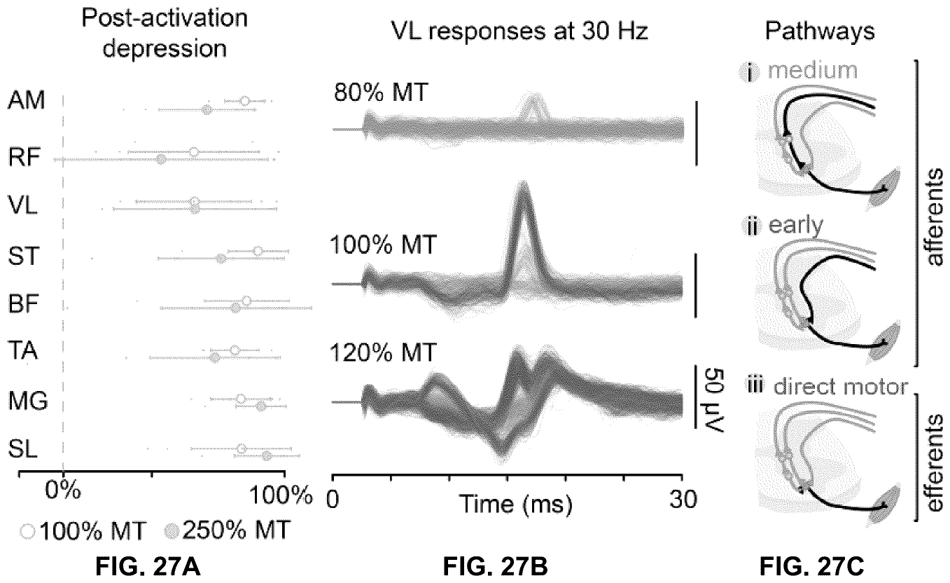

Muscle recruitment by different stimulation amplitudes is evaluated by the degree of post-activation depression to double-pulse stimulation at different stimulation amplitudes (FIG. 27A) and response latencies during continuous 30 Hz stimulation (FIG. 27B). To understand which neural structures are recruited by each stimulation intensity, response latencies during continuous stimulation are compared to those elicited by single-pulse stimulation. Using this approach, one can understand how the manifestation of different types of responses relates to stimulation amplitude as a function of motor threshold.

### Motor Performance.

Motor performance at different stimulation amplitudes is evaluated by the degree of voluntary torque generation on an isokinetic dynamometer. Continuous stimulation is delivered at 30 Hz while the stimulation amplitude is gradually increased to identify the stimulation amplitude (as a function of motor threshold) that enables a 5% increase in maximum voluntary contraction in joints with residual motor function or an increase of 2 standard deviations above baseline noise in joints without residual function.

### Evaluation of Discomfort and Pain.

A combination of self-reports and physiological measurements are employed to evaluate discomfort and pain with each stimulation paradigm. During all stimulation conditions, real-time quantifications of pain are acquired using a PMD-200 Monitor (Medisense, Israel) continuous non-invasive nociception monitoring. This device uses a combination of nociception-related parameters, including heart rate, heart rate variability, pulse wave amplitude, level and fluctuations of galvanic skin response, skin temperature, and movement, to derive the Nociception Level (NOL) index as a real-time measure of nociception. In addition, the Neuropathic Symptom Inventory (NPSI) is used to evaluate qualitative sensations of pain under each stimulation condition. Although this measure is typically used for patients with chronic neuropathic pain, its utility in assessing short-term, immediate changes in patient-reported pain symptoms has been demonstrated.

### Data Analysis

The primary outcomes are voluntary torque production and nociceptive response, assessed through the Nociception Level (NOL) index, across different stimulation amplitude step magnitudes and rates of change. Linear mixed-effects models evaluate the main and interaction effects of amplitude and rate of change on these outcomes. Secondary outcomes include EMG response type, amplitude, and latency. EMG signals are segmented and classified based on electrophysiological features. Changes in the frequency of reflex- and motor-mediated response types across stimulation amplitudes are modeled using the same repeated-measures framework. Additional exploratory analyses may examine patient-reported pain patterns, such as NPSI scores. Covariates, missing data handling, and power assumptions follow a similar rationale as described above in previous examples.

There are distinct types of muscle responses—reflex-mediated medium and early latency responses and motor-mediated short latency responses—that are differentially recruited at varying stimulation amplitudes. Specifically, medium and early latency reflex-mediated responses predominantly occur at stimulation amplitudes below motor threshold, while short latency direct motor responses appear at stimulation amplitudes above motor threshold. Neuroprosthetic, motor-enhancing effect of tSCS on torque generation, as well as evoked responses, appear at stimulation amplitudes close to but below the motor threshold. Thirdly, both the magnitude of the immediate step change in stimulation amplitude and the rate of change in amplitude significantly influence the NOL index. Specifically, larger amplitude steps and faster rates of change result in higher NOL indices, reflecting greater nociceptive response.

### Alternative Stimulation Strategies.

Biphasic stimulation pulses filled with kilohertz frequency (KHF) carriers 5-10 kHz have been thought to suppress the sensitivity of pain receptors and be more comfortable for human participants, allowing them to tolerate higher stimulation amplitudes. However, previous reports showed that KHF waveforms require higher stimulation amplitudes to elicit responses of equal magnitudes as conventional waveforms, so that controlling for response amplitude negates improvements in tolerance by KHF waveforms. Moreover, it was observed that these waveforms negatively impact the recruitment of proprioceptive afferents compared to conventional waveforms (FIG. 20). Therefore, unmodulated, conventional biphasic waveforms are used as the primary approach for this study. However, if stimulation intensity tolerance is a recurring problem, KHF waveforms can be explored as an alternative approach.

### Stimulation Washout Period.

Spinal cord stimulation provides an immediate neuroprosthetic effect that is present while the stimulation is on and is lost when the stimulation is turned off. However, whether electrical stimulation of spinal circuits can promote plasticity by creating a plasticity-permissive environment remains an open question. The time course for a washout period for potential aftereffects of SCS has not been thoroughly documented. However, previous studies in rats, non-human primates, and humans have been able to test different stimulation amplitudes, frequencies, and timings on different joints within a single session. Therefore, it is believed that leaving 1 day between testing sessions should be enough to avoid aftereffects of SCS carrying over across sessions, and statistical comparisons will be made within sessions.

### Future Directions

Conventional applications of tSCS use 30 Hz stimulation with a single cathode over the T11/T12 vertebra with return anodes at the iliac crest or abdomen. The systems, devices, and methods described herein transform the use of tSCS into rehabilitation by providing evidence-based knowledge of how clinicians can tune stimulation location, frequency, and/or amplitude to enable different types of movements. Enabling the targeting of individual muscle groups with non-invasive tSCS has the potential to improve movements used in rehabilitation exercises by preventing co-contractions with antagonist muscles. Therefore, the approach can enable the practice of dexterous tasks involving multiple muscles that are sequentially activated throughout different phases of movement, such as cycling and walking.

Given the potential interactions between spatial, frequency, and amplitude stimulation parameters, their modulatory effects are independently studied. In some aspects, preliminary data can be collected to investigate the combined effects of spatiotemporal tSCS control on a functional, rhythmic task-leg cycling on a stationary leg ergometer bike. This allows for the evaluation of the feasibility of real-time parameter adjustments in tSCS to support coordinated multi-joint movements, providing preliminary data for future translation into a clinical trial on spatiotemporal tSCS during bodyweight-supported locomotion training

What is claimed is:

1. A method of transcutaneous spinal cord stimulation of a subject, the method comprising:
a. providing a multielectrode stimulation device comprising at least two transdermal spinal cord stimulation (tSCS) electrodes and at least one return electrode;
b. positioning a first and second tSCS electrode of the at least two tSCS electrodes on the back of the subject at a symmetrical lateral separation distance from a midline of the patient and at a shared rostrocaudal position of the patient, respectively, wherein the positioning is selected to activate at least one motor neuron pool;
c. positioning the at least one return electrode on an abdomen of the subject; and
d. activating the at least one specific motor neuron pool to stimulate at least one specific muscle group using a predetermined stimulation pattern, the predetermined stimulation pattern comprising a temporal schedule of at least one of a stimulation voltage or a stimulation amperage for each of the at least two tSCS electrodes.

2. The method of claim 1, wherein the predetermined stimulation pattern further includes at least one of a stimulation frequency and a stimulation amplitude.

3. The method of claim 1, wherein the temporal schedule of stimulation voltages or amperages comprises:
a. increasing the stimulation amplitude in all the tSCS electrodes at a baseline rate to a baseline stimulation amplitude;
b. maintaining all the tSCS electrodes at the baseline stimulation amplitude; and
c. for at least one of the tSCS electrodes, increasing the stimulation amplitude at an activation rate from the baseline stimulation amplitude to an activation amplitude, maintaining the activation amplitude for a stimulation period, and decreasing the stimulation amplitude back to the baseline stimulation amplitude at a deactivation rate;
wherein the baseline rate is slower than either the activation rate or the deactivation rate.

4. The method of claim 3, wherein the baseline stimulation amplitude ranges from about 50% to about 95% of the motor threshold amplitude, wherein the motor threshold amplitude comprises the stimulation amplitude that induces a peak-to-peak response amplitude of at least about 20 UV within a latency ranging from about 10 to about 30 ms in any muscle.

5. The method of claim 4, wherein:
a. the baseline rate comprises increasing the stimulation amplitude from zero to the baseline stimulation amplitude over a habituation period ranging from about ten seconds to about ten minutes;
b. the activation rate comprises increasing the stimulation amplitude from the baseline stimulation amplitude to the activation amplitude over an activation period ranging from instantaneous to about 1 minute; and
c. the deactivation rate comprises decreasing the stimulation amplitude from the activation amplitude to the baseline stimulation amplitude over a deactivation period ranging from instantaneous to about 1 minute.

6. The method of claim 4, wherein the activation amplitude ranges from the motor threshold amplitude to a saturation amplitude, wherein the saturation amplitude comprises:
a. a stimulation amplitude limit at which no additional increase in a response amplitude of the first recruited muscles is observed; or
b. a maximum stimulation amplitude tolerated by the subject.

7. The method of claim 6, wherein the at least two electrodes may be activated singularly or in combination with any of the other electrodes.

8. The method of claim 6, wherein the at least two electrodes are activated sequentially.

9. The method of claim 2, wherein the predetermined stimulation pattern comprises:
a. the stimulation frequency selected to selectively target a flexor or extensor muscle group, wherein:
i. the stimulation frequency ranging from about 20 Hz to about 40 Hz selectively targets the extensor muscle group; and
ii. the stimulation frequency ranging from about 40 Hz to about 100 Hz selectively targets the flexor muscle group; and
b. the activation amplitude selected to recruit a portion of the flexor or extensor muscle group.

10. The method of claim 1, wherein the at least two tSCS electrodes of the multielectrode stimulation device are positioned at a vertebral segment selected from:
a. a thoracic or lumbar vertebral segment for assisting lower limb movements; or
b. a cervical vertebral segment for assisting upper limb movements.

11. The method of claim 10, wherein:
a. the thoracic or lumbar vertebral segment is selected from a vertebral segment ranging from T9 to L1; and
b. the cervical vertebral segment is selected from a vertebral segment ranging from C3 to T1.

12. The method of claim 1, wherein the activation of a specific muscle group is determined using a selectivity index (SI) and a recruitment probability, wherein:
a. the selectivity index SI of muscle m is a difference between a normalized recruitment of muscle m (RECₘ) and an average normalized recruitment of M−1 muscles, excluding the muscle, m (RECₙ), as expressed by:
$${SI}_{m} = {REC}_{m} - \frac{{\sum }_{n \neq m}^{M} {REC}_{n}}{M - 1} ;$$
and
b. the recruitment probability estimates an activation of motoneuron pools in each spinal segment (Sᵢ) as a linear combination of each normalized muscle recruitment Mⱼ and Wᵢⱼ, an expected segmental distribution of motoneuron pools innervating muscle j at spinal segment Sᵢ, as expressed by:
$${S}_{i} = \frac{{\sum }_{muscles} {W}_{i , j} {M}_{j}}{{\sum }_{muscles} {W}_{i , j}} .$$

13. The method of claim 1, wherein the specific muscle groups targeted are selected from hip muscle groups, leg muscle groups, ankle muscle groups, shoulder muscle groups, arm muscle groups, forearm muscle groups, hand muscle groups, and any combination thereof.

14. The method of claim 1, further comprising altering the placement of the at least two transdermal spinal cord stimulation (tSCS) electrodes to provide selectivity for different muscle groups.

15. A non-invasive brain-spine interface (BSI) system to facilitate movements in a subject with a spinal cord injury (SCI), the system comprising:
a. a wearable electroencephalogram EEG device comprising an array of surface EEG electrodes configured to detect a plurality of signals indicative of brain activity in the brain of the subject;
b. a multielectrode transcutaneous spinal cord stimulation (tSCS) device comprising an array of tSCS electrodes configured for placement over a predetermined region of the subject's back and a pair of return electrodes positioned over a left and right region of the subject's abdomen, wherein the predetermined region is selected to activate at least one motor neuron pool that stimulates at least one specific muscle group; and
c. a computing device operatively coupled to the wearable EEG device and the tSCS device, the computing device comprising at least one processor configured to:
i. receive the plurality of signals from the wearable EEG device;
ii. identify a movement intention by the subject based on the plurality of signals using a machine learning model and producing a movement intention signal; and
iii. activate the tSCS device to produce stimulations using a predetermined stimulation pattern, the predetermined stimulation pattern comprising a temporal schedule of at least one of a stimulation voltage or a stimulation amperage for each of the at least two tSCS electrodes, predetermined stimulation pattern further comprising a predetermined series of voltages or amperages delivered to the subject through individual stimulation electrodes from the array of stimulation electrodes, the stimulation pattern configured to stimulate muscle activation patterns, wherein the muscle activation patterns are configured to facilitate movements of the subject.

16. The system of claim 15, wherein the wearable electroencephalogram EEG device comprises an array of 32 surface EEG electrodes.

17. The system of claim 15, wherein the array of surface EEG electrodes is configured to be positioned over a sensorimotor cortex of the subject.

18. The system of claim 15, wherein the machine learning model comprises a linear discriminant analysis (LDA) classifier model.

19. The system of claim 15, wherein the at least one processor is further configured to train the machine learning model using a training dataset comprising a plurality of EEG signal sets recording using the EEG device, wherein each EEG signal set is associated with an intended movement of the subject.

20. The system of claim 15, wherein the predetermined stimulation pattern further includes at least one of a stimulation frequency and a stimulation amplitude, wherein:
a. the stimulation frequency is selected to selectively target a flexor or extensor muscle group, wherein, the stimulation frequency ranging from about 20 Hz to about 40 Hz selectively targets the extensor muscle group and the stimulation frequency ranging from about 40 Hz to about 100 Hz selectively targets the flexor muscle group; and
b. the stimulation amplitude ranges from a motor threshold amplitude to a saturation amplitude, wherein:
i. the motor threshold amplitude comprises the stimulation amplitude that induces a peak-to-peak response amplitude of at least about 20 μV within a latency ranging from about 10 to about 30 ms in any muscle; and
ii. the saturation amplitude comprises a stimulation amplitude limit at which no additional increase in a response amplitude of the first recruited muscles is observed; or a maximum stimulation amplitude tolerated by the subject.

21. The system of claim 19, wherein the predetermined stimulation pattern further includes a habituation to a baseline stimulation amplitude, wherein the baseline stimulation amplitude ranges from about 50% to about 95% of the motor threshold amplitude.
